(19) United States
(12) Patent Application Publication
(10) Pub. No.: US 2023/0414637 A1
(43) Pub. Date: Dec. 28, 2023

(54) NICLOSAMIDE NASAL AND THROAT SPRAY

(71) Applicant: NEEDHAM MATERIAL SCIENCE LLC, Durham, NC (US)

(72) Inventor: David NEEDHAM, Durham, NC (US)

(21) Appl. No.: 18/252,277

(22) PCT Filed: Nov. 9, 2021
(86) PCT No.: PCT/US2021/072303

§ 371 Date: May 9, 2023

Related U.S. Application Data
(60) Provisional application No. 63/227,408, filed on Jul. 30, 2021.
(60) Provisional application No. 63/111,184, filed on Nov. 9, 2020.

(51) Int. Cl. A61K 31/609; A61K 47/38; A61K 47/10; A61P 31/14; A61K 9/00

(52) U.S. Cl. CPC A61K 31/609; A61K 47/38; A61K 47/10; A61P 31/14; A61K 9/0043

(57) ABSTRACT

The present disclosure provides prophylactic nasal and throat sprays that can be used to prevent initial infection of COVID19, its more contagious variants, and other respiratory viral infections, and the methods of making of the same. Data is given in a series of embodiments that show how niclosamide from one supplier (AK Sci), who's initial powder has a block-like morphology of what appears to be a high solubility polymorph, readily dissolves in pH buffers providing increased amounts in solution as the solution pH is increased. Unlike more conventional particulate drug materials, this provides 20 uM to 30 uM solutions of niclosamide at pH 8 for a prophylactic, preventative nasal spray that has rapid permeation through the nasal mucin and direct access to the underlying epithelial cells that the virus infects. At higher pH of 9.2 it can provide a 300 uM solution of niclosamide for throat spray to be used at first signs of early infection. Another embodiment shows that niclosamide from a second supplier (Sigma), that already contains some of the low solubility most stable polymorph (the monohydrate) gives an initial dissolution (3 hrs) that is similar to the AK Sci material, overnight stirring converts the powdered excess niclosamide to the more stable and lower solubility polymorph. A further embodiment shows how niclosamide from commercially available tablets (Yomesan) can be extracted into aqueous pH solution that does not involve the use of organic solvents. Additional embodiments show a solvent injection technique can be used to make supersaturated solutions of niclosamide that eventually precipitate and also form the low solubility monohydrate polymorph as do water, acetone, and ethanol niclosamide cosolvates. The solvent injection technique is the preferred method to make niclosamide solutions at concentrations where it is soluble because the initial powdered material can be dissolved in ethanol, any microparticulate impurities filtered out, and a clear solution made that is not in contact or equilibrium with any powdered material, that if it forms the monohydrate polymorph would reduce the amount of niclosamide in solution and therefore reduce its bioavailability to the nasal and respiratory epithelium.

### FIELD OF THE INVENTION

The present disclosure provides prophylactic pH-buffered solution of niclosamide that can be used as a nasal and throat spray to prevent initial infection of COVID19, its more contagious variants, and other respiratory viral infections. It also provides a method of making such solution that does not require excess niclosamide to be dissolved in aqueous media. Furthermore, it describes a method for extracting niclosamide from commercially available tablets into aqueous solution that does not involve the use of organic solvents.

### BACKGROUND OF THE INVENTION

This patent application lays out the case for a potentially new prophylactic/preventative and early treatment for COVID19, its more contagious variants (including the Delta variant [7]), and other respiratory viral infections. It is based on the anti-helminthic drug niclosamide, marketed as Yomesan by Bayer [8] and other generics, that has been routinely given to humans for the past 60 years as oral tablets in a 2-gram dose to cure cestode parasites (tape worms) [9]. As an oral dose of “well-chewed” tablets it is deemed very safe. According to the World health Organization, niclosamide is of very low toxicity to mammals (WHO Hazard Class III) [10], with just slight digestive disorder, and no systemic therapeutic effects [8]. Its acute oral toxicity is >1000 mg/kg [11], i.e., a 70 kg person would have to ingest 70 grams of the niclosamide tablets to show severe GI symptoms.

We have much previous experience working with and preparing niclosamide for drug delivery, including as a prodrug therapeutic for anti-cancer applications [12, 13]. Following publication of the bioRxiv preprint by Jeon et al in March 2020 [14] (now peer-reviewed [15]), that demonstrated anti-viral activity of niclosamide against SARS-CoV-2, our efforts were refocused on ways to increase the amount of niclosamide in sprayable nasal and throat suspensions [16]. A preformulation drug characterization of niclosamide (pKa, water-solubility, and Log P) was carried out along with an experimental evaluation of the amount of niclosamide that could be dissolved in aqueous solution as a function of pH. Results revealed that the concentration of niclosamide in aqueous solution can be increased by simply increasing solution pH by only one to two logs, into the pH 8-9 range. However, studies also showed that “niclosamide is not niclosamide is not niclosamide”, i.e., in the presence of solid phase niclosamide, the amount of niclosamide in solution is determined by the polymorphic nature of the material [17] that the aqueous solution is in equilibrium with or is moving towards. These polymorphic forms are different from supplier to supplier as shown here for AKSci (CA) and Sigma products. Such solid phase material includes precipitated or particulate niclosamide as micronized or stabilized microparticles. Our lead niclosamide formulation is therefore a simple buffered solution that represents a low dose prophylactic nasal spray that could stop virus replication at its point of entry, and a higher concentration throat spray, that could reduce viral load as it progresses down the back of the throat.

### 1.1. Niclosamide Turns Down the Cell's Dimmer Switch Such that the Virus Can't Replicate

As discussed in detail below (1.2 Why Niclosamide?), niclosamide, the molecule, has emerged from multiple drug screens as a very interesting compound; not just as a pesticide but also with potential for cancer and many other diseases and conditions, and now in a broad range of viral infections [14, 18-32]. The reason for this wide-ranging activity is that one of its main effects is in mitochondria, where it reduces the cell's production of its main energy molecule, adenosine triphosphate (ATP). ATP is upstream of many key cellular processes including transcription and translation of the virus RNA. ATP is also a substrate for the multi-subunit enzyme RNA polymerase that adds ATP and the other ribonucleotides to a growing RNA strand. As described by Zimmerman et al, in the book Cellular Respiration, [33], ATP also serves as a cofactor for signal transduction reactions using a variety of kinases as well as adenyl cyclase. Hence, its production and presence in every cell is essential to their functioning on multiple levels¹. Normally, cellular ATP concentration is maintained in the range of 1 to 10 mmol/L. However, by reducing the ATP content, by titrating it with niclosamide, basically turning down the dimmer switch on the cell's energy production, niclosamide can inhibit virus replication in the ubiquitously used Vero 6 cells at only 1 uM [14] and has also been shown to have similar anti-replicative efficacy in cultured Calu-3 lung cells, at 2 uM [34]. (We still need data on actual airway epithelial cells). ¹ This fact is fascinating and puts the importance of ATP in context. According to the review by Zimmerman et al, “Approximately, 100 to 150 mol/L of ATP are required daily, which means that each ATP molecule is recycled some 1000 to 1500 times per day. Basically, the human body turns over its weight in ATP daily. 39 Transmembrane proton flux through the mitochondrial ATPase synthase complex occurs at an estimated rate of 3×10²¹ protons per second. This corresponds to ATP reformed at a rate of 9×10²⁰ molecules/sec, or approximately 65 kg ATP recycled per day in a normal resting adult”. “Inadequate ATP impairs the translation of cellular structure to cellular function, a defining characteristic of life”.

Because of this quite dramatic efficacious effect, and the urgency of the COVID19 pandemic, in addition to repurposing the original oral tablets, there have been efforts to develop new formulations and routes of administration for niclosamide, to validate them in preclinical studies, and clinically test niclosamide for COVID-19 [35]. These include new intramuscular injections [36], and of particular interest here is an inhalant as a spray-dried-lysozyme particulate of micronized-niclosamide that has entered a 1,500 high-risk kidney patient study in the UK [37].

The main formulation-versus-efficacy-issue with any formulation, especially locally in the nose and throat, is based around the fact that, while its efficacy is 2 μM [34] niclosamide's solubility in aqueous media at neutral or lower pH, as encountered in the nasal pharynx, is of the same order or perhaps slightly less, i.e., ˜1 μM-2 μM. Since any microparticle formulation cannot deliver drug directly to the epithelial cells where initial infection takes place because of the protective mucin barrier that is produced by and covers these cells, the only active and bioavailable species of any microparticle suspension will be the soluble fraction of niclosamide in any delivered solution. It is here that we have found a way to increase the solubility of niclosamide in simple aqueous buffered solutions that are still within the natural pH range for the nasopharynx as a prophylactic nasal spray and tolerable for the oropharynx as an early treatment throat spray.

### 1.2 Repurposing Niclosamide

Since 1950, there are only 1,167 entries in PubMed for the word “niclosamide” [38]. Niclosamide arrived as a molluscicide in 1950, and then as a treatment for worms in and around 1960-61 when 13 papers were published led by Bayer and colleagues. Thus, from 1950 until 2010 the average number of publications was 11 per yr., +/−4.6 and they were mostly concerned with the pharmacology and also environmental impact of niclosamide in this context as an oxidative phosphorylation inhibitor. However, as niclosamide started to emerge in many independent drug screens as a potential therapeutic molecule, especially for cancer, and now viral infections, the number of “niclosamide” publications has increased on a yearly basis from 26 in 2011, to 90, 82, 79, and 76 in 2018-2021 respectively. We therefore see this molecule and others like it, (proton shunts) [39] as quite scientifically rich and therapeutically impactful for new investigations and indeed new investigators looking for interesting and meaningful projects.

This document has therefore also been written to generate scientific interest in niclosamide, to provide new knowledge especially about its pH dependence of solubility to inform current and future formulations, and to encourage other researchers to join us and submit their own proposals in their own specialized models for evaluating the efficacy and safety of niclosamide solutions as described and made herein. To this end, section 6 lays out a series of hypotheses to be tested including a brief summary of our own proposed preclinical studies that are given to again encourage others to work in this area, not just in viral infections, but also in any area that niclosamide could have a beneficial therapeutic effect; for example we are also exploring its efficacy in cancer as a prodrug therapeutic [13]. The hope is that this patent application will generate interest and invite partnerships from national governments including beyond the USA where niclosamide is still approved, infectious disease institutes and perhaps companies with the wherewithal to test it in cell and animal studies and move this simple solution formulation on to IND and clinical trials so that it can be rapidly adopted for COVID19 and, indeed, other respiratory infections.

### SUMMARY OF THE INVENTION

This invention brings together a series of physical chemical, physiological, cell biological, molecular biological, viral, and clinical aspects for a particular drug, niclosamide. It addresses and overcomes serious limitations for this particular drug's solubility and availability in aqueous solution to create a prophylactic preventative nasal spray to combat initial COVID infection. The traditional route for nasal spray therapeutics, especially for low solubility drugs, is to micronize the material, as in Flonase and Nasonex. Here, these drugs fluticasone propionate and mometasone furoate have similarly low solubilities to niclosamide in the 10s of micromolar but are active at 1000 times lower than this solubility, in the 10s of nanomolar. Therefore, while their micro particles cannot pass through the nasal mucus, the amount that dissolves in solution, while still relatively low, can permeate the mucus as a solution at amounts that are far in excess than the therapeutic levels required to have efficacy.

In contrast, regarding the efficacy of niclosamide, there is overwhelming evidence in the literature that, in extended (24 to 48 hrs) cell culture, niclosamide can reduce the energy the virus needs for replication and that the virus can be prevented from replicating at levels of 2-3 uM niclosamide, However, the issue for developing and using a clinically effective formulation is that the aqueous intrinsic solubility of niclosamide is so low (˜1 uM) that, at nasal pH (˜6-7), the amount of niclosamide in solution (1 uM-2 uM) that is in equilibrium with micronized material is at the same level or less than the amount required to inhibit that viral replication of 2 uM-3 uM.

Here the case is laid out for a new prophylactic nasal and early treatment throat sprays for COVID19, its more contagious variants [7], and other respiratory viral infections. The spray solutions are based on the drug niclosamide, a broad spectrum virostatic host cell modulator that is, in principle, specific for every virally infected or infectable cell. Niclosamide has already shown convincing activity against SARS-CoV-2 [40-44] and other viruses [32]. At the cellular level, of the six basic stages that are essential for viral replication, niclosamide can inhibit three of these, namely, 3. Uncoating, preventing RNA release from the endosome); 4. Replication, reducing the amount of ATP available from mitochondria for viral transcription and translation; and 5. Assembly in the Golgi that then promotes the secretion of non-competent virions. The main issue with niclosamide though is its low water solubility and therefore low bioavailability at mucin-covered epithelial surfaces. While the complete inhibition of viral replication in Calu-3 cells is an estimated 2 uM-3 uM [18, 34], niclosamide only has a water solubility of 1 uM-2 uM at nasal pH and so this motivates the need for higher concentrations of niclosamide in solution.

Experimental: Experimentation focused on four main specific aims: 1) To measure the pH dependence of Niclosamide concentration in buffered solutions using UV/Vis spectroscopy for niclosamide form different suppliers AKSci and Sigma (and therefore different initial dry powder polymorphs) as well as after precipitation from supersaturated solution and recrystallization as water, acetone and ethanol cosolvates; 2) and compare the data to predictions from Henderson Hasselbalch and precipitation pH models for the measured intrinsic solubilities of the protonated acid polymorphs and obtain a fitted pKa; 3) To measure dissolution rates of niclosamide for each of the forms of niclosamide as a function of pH including a new methodology for measuring the supernatant concentration in situ; and 4) To extract niclosamide from commercially available and regulatory-approved tablets such that the obtained niclosamide would be very close to an already approved dosage composition; 5) to observe the excess undissolved material that was in equilibrium with the supernatant solutions by bright filed optical microscopy.

Results: When excess powdered niclosamide from two different suppliers (AKSci and Sigma) was dissolved in pH buffers, supernatant niclosamide concentration increased slowly over the lower pH range from 3.66 to just above 8, but then showed a more rapid rise in concentration from 8.5 to 9.5, reaching over 700 uM at pH 9.63. However, after overnight stirring the excess undissolved Sigma niclosamide that was in equilibrium with the supernatant converted to the low solubility monohydrate polymorph while the excess AKSci material retained its initial powder morphology and its high solubility. This was evident from optical microscopy images of the two samples showing the original block like morphology of the AKSci material while the block and spike morphology of the Sigma material had all converted to the spiky monohydrate morphology.

In the dissolution experiments, for a nominal 0.5 mM-1 mM niclosamide, the fitted logarithmic rate for niclosamide dissolution over a pH range from 8.62 to 9.44 increased by a factor of just over 3×. A more optimized stirring gave a further increased rate of dissolution as expected from dissolution models. Dissolution rates for the water, acetone, and ethanol cosolvates were slower than for the AKSci and Sigma powders commensurate with their lower intrinsic solubilities. Niclosamide was also readily extracted from Yomesan tablets at an overall rate of dissolution comparable to that of the AK Sci niclosamide powder reaching a stable niclosamide concentration of 348.3 uM at pH 9.34.

Conclusion: Comparison between data and theory showed that the theoretical maximum amount of niclosamide in aqueous solution is determined by the relative amounts of its pH-dependent protonated and unprotonated forms and follows the Henderson-Hasselbalch and precipitation-pH (pHp) models. This pH-dependent behavior provides a mechanism for increasing the bioavailability of niclosamide in mucin-penetrating solutions for direct application to the mucin-covered epithelia as nasal and throat sprays. Niclosamide form different suppliers has different initial polymorphic forms and so caution is warranted in order to provide the highest intrinsic solubility form to any niclosamide formulations especially for nasal administration since the monohydrate at pH6 only has a solubility ˜1 uM and the efficacy for inhibiting viral replication is slightly higher at 2-3 uM niclosamide.

Based on our finding we propose a low dose prophylactic nasal spray (20 uM to 30 uM niclosamide solution at pH 8.0) that could stop the virus at its point of entry and a higher concentration throat spray (300 uM solution at pH 9.2) that could reduce viral load as infected cells and virus progress down the back of the throat.

Niclosamide has emerged as a very interesting and potentially successful ubiquitous anti-viral virostatic that could be used in conjunction with all other treatments of COVID19 and other viral infections. The simple niclosamide solutions developed here are optimized to penetrate the mucus delivering niclosamide concentrations that are 10-20× the expected concentrations required to completely inhibit viral replication, and ell as inhibit viral entry into nasal epithelial cells and promote the secretion of inactive virions. This spray then could have a significant impact in this regard since the presymptomatic or early stage of the disease is a key time point to prevent further SARS-CoV-2 spread in both unvaccinated and vaccinated populations.

### BRIEF DESCRIPTION OF THE FIGURES

FIG. 1. Niclosamide (5-Chloro-N-(2-chloro-4-nitrophenyl)-2-hydroxybenz-amide) contains an ionizable OH and a zwitterionic nitro group. It is the ionizable group that provides for the much higher aqueous solubility as a function of pH.

FIG. 2. The Henderson-Hasselbalch equation calculates the fractions of each species as a function of pH, shown here for a nominal pKa (derived from experiment) of 7.12.

FIG. 3. The effects of two parameters: Precipitation pH and pKa of niclosamide on its solubility limits.
FIG. 3A Precipitation pH versus Concentration of Niclosamide (μM) as a function of pKₐ (5.5-7.0) and constant nominal solubility of Nic_{OH}, Sₒ of 1 μM.
FIG. 3B Precipitation pH versus concentration of Niclosamide (μM) as a function of solubility of Nic_{OH}, Sₒ, (0.25 μM-5.0 μM) and constant nominal pKₐ of 6.87.

FIG. 4. Comparison between HH curves and pHp for niclosamide as a function of solution pH for pKₐ=7.12, Sₒ=2.53 uM.

FIG. 5. 1 mM Precipitated Niclosamide forms a visible fluffy precipitate, especially when shaken, that would be unsuitable for a nasal spray [3].

FIG. 6. eVol syringe mounted in drill press, with 20 mL scintillation vial and magnetic stirrer set up, showing the simple procedure for introducing microliters of ethanolic niclosamide into the stirred vial.

FIG. 7. Photographic image of the series of calibration standards as 10 mL samples that (when viewed in color) show increasing yellowness of the samples with increasing niclosamide concentration from 25 uM to 300 uM all at pH 9.3 TRIZMA buffer.

FIG. 8. Series of UV/VIS spectra at pH 9.3 and 20° C. for each niclosamide solution corresponding to the photographic images of the vials in FIG. 7. The absorbance of the peak at 333 nm was used in the calibration (although the maximum in aqueous pH 9.3 buffer was closer to 335 nm).

FIG. 9. UV/VIS Calibration for niclosamide solutions at 20° C. Averaged UV/VIS Absorbance at 333 nm for Niclosamide Solutions measured by Nanodrop. Data labels give the absorbance at each niclosamide FIG. 10. 10 mL samples of excess niclosamide dissolved in pH buffers (nominally 7.0-9.3). Also shown is a deionized water sample (far left) (nominally pH 6.22) and a 300 uM Niclosamide in a pH 9.3 standard (far right), made by solvent injection (measured to be 301 uM±5 uM), showing (when viewed in color) the increasing “yellowness” characteristic of niclosamide in solution.

FIG. 11A. Equilibrated supernatant concentrations for dissolution of powdered niclosamide (from AK Sci) added in excess to each pH buffer at 20° C. Plotted is the Supernatant pH versus the Supernatant Niclosamide concentration [Nic] (uM) measured by UV5 Nano (Mettler Toledo) UV/Vis and compared to the pHp curve Eqn 4. Plotting this data against the pHp predictions for a measured intrinsic solubility for AKSci niclosamide So1 of 2.53 uM, gives a fitted pKa for niclosamide of 7.12.

FIG. 11B. Same data as in FIG. 11A but axes changed to perhaps a more easily evaluated form. Plotted is the Supernatant Nic concentration (UV/VIS) versus supernatant pH at 20° C. Also included is the pHp theory for pKₐ of 7.12 and limiting Nic_{OH} solubility of 2.53 uM at pH 3.66. As shown on the graph, a 20 uM prophylactic solution can be made at pH 7.96; a 200 uM early treatment throat spray can be made at pH 9.01, and the concentration can be raised to 300 uM at pH 9.19. The amount of Niclosamide in solution can increase to 703.6 uM at pH 9.63.

FIG. 12. Photographic images from the Optical microscope of AK Sci powder after grinding with a mortar and pestle, resuspending in water to disperse, and bath sonicating to help break up aggregates. Shown are typical block-like particles as separate or in aggregated clumps of material. This is in sharp contrast to monohydrates that are typically spiky, needle-shaped, and rod-like in appearance as shown in the literature by van Tonder et al [5] as well as in subsequent images in this study (see FIGS. 20, 23, 26 and 27). (Bright field, 40× objective lens, with Köhler illumination)

FIG. 13. Dissolution curves for the different pHs using the nanodrop UV/Vis spectrometer (ThermoFisher) over the first 1 hr after addition of dry powdered AK Sci niclosamide. Data is given for the higher pH range 8.62-9.5 for a nominal total 1 mM of the AK Sci material. The top two curves, show the (empirical) effect of more optimized stirring, that increases the dissolution rate by ˜33%, represented by AK Sci and a second supplier of niclosamide, Sigma. Also shown is dissolution of Yomesan tablet powder in pH 9.33. AK Sci niclosamide reached 564 uM at 3 hrs and Sigma niclosamide reached 680 uM at 3 hrs.

FIG. 14. Grey bars represent Initial rates for dissolution as uM/mg·s over the first 3 mins of the plots in FIG. 13, for AK Sci niclosamide into five different pH solutions of pH 8.62, 8.72, 9.06, 9.36 and 9.5. Bars in black represent the effect of a more optimized stirring for AK Sci at pH 9.5 and a niclosamide from a second supplier, Sigma at the same pH. Also shown is the initial rate of dissolution of niclosamide from the ground Yomesan tablet powder.

FIG. 15. Initial rates of niclosamide dissolution Supernatant [Nic] (uM/mg) versus Supernatant niclosamide concentration [Nic] (uM) at equilibrium (from the data in FIG. 11B) (see FIG. 18 for raw data for AKSci and extraction from crushed Yomesan tablets).

FIG. 16. Dissolution of ground Yomesan powder using the optimized stirring protocol. The sample became progressively cloudy as excipient tablet material rapidly hydrated and was stirred into suspension. The niclosamide concentration of the filtered sample was 348.3 uM.

FIG. 17. Extraction of Niclosamide from Yomesan, filtered at 75 minutes to recover a stable solution. A) clear supernatant solution of 520 uM AK Sci Niclosamide and 435 uM Niclosamide from Yomesan tablets as measured for the cloudy suspension; B) 520 mM AK Sci Niclosamide and 348 uM Niclosamide from Yomesan tablets after filtering (0.22 mm filter), as measured for the clear solution. (Images are better when viewed in color).

FIG. 18. Initial Dissolution of (left) AKSci Niclosamide and (right) Yomesan Niclosamide in pH 9.3 buffer. AKSci niclosamide is slightly faster at 51.7 uM/min and niclosamide from Yomesan at 43.1 uM/min. These convert to 0.28 ug/s and 0.23 ug/s respectively for AKSci niclosamide and niclosamide from Yomesan powders

FIG. 19 Supernatant Niclosamide concentrations [Nic] (uM) versus supernatant pH for Niclosamide precipitated from supersaturated solution (open circles) and pHp theory with pKₐ=7.12, Sₒ2=1.0 uM (dashed line) compared to AK Sci Niclosamide dissolved from powder (filled symbols) with the same pKₐ=7.12, and Sₒ1=2.53 uM (solid line).

FIG. 20. FIG. 20 Niclosamide precipitated and equilibrated from supersaturated solutions form a series of niclosamide morphologies of reduced solubility that change with pH. Shown are the optical microscope images (bright field, 40× objective lens, with Köhler illumination) taken from the precipitated samples at each indicated pH. Scale bar is 10 uM.

FIG. 21. The niclosamide gel-like particles also display a strong hydrophobicity. Gas bubbles, that were adsorbed or trapped in the microparticles due to the stirring during initial mixing and subsequent stirred equilibration, follow the crumpled contours of the niclosamide precipitated sheet formed at pHs 7.0, 7.5, 8.0 and 8.5. The precipitate adheres to the bubble surface showing that the interfacial tension and Laplace pressure are zero. (Bright field, 40× objective lens, with Köhler illumination).

FIG. 22. Dissolution of Niclosamide (H₂O precipitate and recrystallized from Acetone and Ethanol) Supernatant Niclosamide concentration [Nic] (uM) versus time (min). Also shown are the logarithmic equations for the fitted overall rates and, in parentheses, are the final equilibrium supernatant concentrations of niclosamide several days after dissolution was started.

FIG. 23. Optical microscope images of Niclosamide precipitated from supersaturation (at 1% ethanol), and Niclosamide recrystallized from ethanol and acetone. (Bright field, 40× objective lens, with Köhler illumination).

FIG. 24. Comparison between (top) Sigma niclosamide freshly dissolved at 3 hrs as clear suspensions and (bottom) after 24 hrs stirring, showing that the clear supernatant was more cloudy suspension.

FIG. 25. Dissolution of Sigma Niclosamide in pH buffer at 3 hrs and 24 hrs: Supernatant Niclosamide [Nic](uM) vs Supernatant pH and the pHp curve (fitted with pKa=7.12 and So2=1.01 uM). The Sigma data (filled triangles) is compared to the AKSci data (filled circles) for the same overnight equilibration. Data for Sigma niclosamide dissolution after 3 hrs is shown in parentheses as an open triangle at pH 9.5 and 681 uM. Also shown are photographic images of the same Sigma niclosamide sample in the 20 mL scintillation vial for the clear supernatant after 3 hr dissolution (clear supernatant at 681 uM) and the after overnight stirring (cloudy supernatant at 247 uM).

FIG. 26. Optical microscope images of (left) the Sigma Niclosamide as received from the supplier and (right) after overnight equilibration at pH 9.3. Overnight equilibration of Sigma Niclosamide (right) produced long rod-like and spiky-bundled morphology and a background of smaller fainter rods that have a much lower (˜3.5×) solubility than the parent Niclosamide powder.

FIG. 27 A. Optical microscope images of excess Sigma Niclosamide powder particles after overnight equilibration over the whole pH range from 3.61 to 9.51. This overnight equilibration produced long rod-like, needle-shaped, and spiky-bundled structures at all pHs tested showing that the Sigma niclosamide converted to the lower solubility “spiky” polymorph as the lower solubility monohydrate. These hydrated samples are compared to the dry, as-received, powder from the supplier that already showed a considerable amount of rod like and needle-shaped crystal morphology in addition to a more block like morphology.

FIG. 27 B. Optical microscope images of excess AKSci Niclosamide powder particles after overnight equilibration over the whole pH range from 3.65 to 9.63. In contrast to the Sigma niclosamide samples, this AKSci material retained its block like morphology at all pHs tested. There was a distinct absence of the prototypical spiky bundled morphology characteristic of the monohydrate. Thus, the AKSci material did not convert to the lower solubility needle-shaped “spiky” monohydrate. These hydrated samples are compared to the dry, as-received, powder from the AKSci supplier that have the block like morphology.

FIG. 28 Supernatant Niclosamide concentrations [Nic] (uM) versus Supernatant pH for Dissolved-Equilibrated Niclosamide from AK Sci and Sigma, and Supersaturated-Precipitated Niclosamide plus the Water Acetone and Ethanol solvates at pH 9.3. Dotted lines show the pHp theory for pKa=7.12 and the AKSci polymorph (So1 solubility=2.53 uM) and presumed monohydrate So2 solubility=1.01 uM.

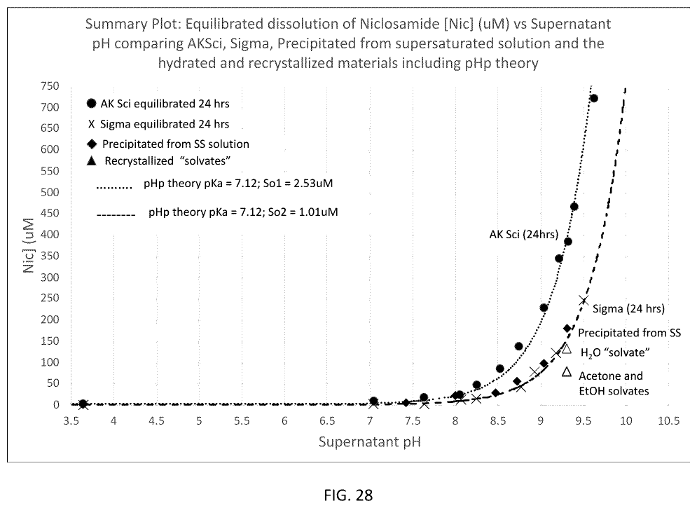

FIG. 29. Pathogenesis and kinetics of coronavirus infections. Top image from Hou et al [1], where the greyish color (originally red) highlights SARS-COV-2-infected ciliated cells in a covid-19 patient's bronchi and an, originally, green dye is an acetylated α-tubulin cilia marker. The second image below this epithelium is adapted and modified from Ocea et al, [2]), showing the six stages of viral infection [4], namely: 1. Attachment, 2. Penetration, 3. Uncoating, 4. Replication, 5. Assembly, and 6. Secretion and virion release. The whole process takes ˜36 hrs [6].

FIG. 30. Pathogenesis and kinetics of coronavirus infections. Top image is again adapted from Hou et al [1], now schematically showing, in more intense grey speckling, the saturation of the mucin with niclosamide solution and the incorporation of niclosamide into the plasma membranes of at least the top layer of epithelial cell. Niclosamide saturation of interior organelles are schematically illustrated in the main cell figure, again adapted, and modified from Docea et al, [2], but now showing the stages that are affected by niclosamide's proton shunt activity. These are: when it enters the lipid bilayer membranes for endosome and mitochondria and Golgi where it dissipates pH gradients and inhibits the following three stages of viral infection: viral 3. Uncoating and RNA release from the endosome; 4. Replication, where it reduces ATP from mitochondria; and viral 5. Assembly in the Golgi and hence 6. Secretion of non-competent virions.

FIG. 31. Schematic of the nasal epithelium with its first layer of cells comparing a micronized niclosamide and a solution of niclosamide with respect to mucin penetration. A) Administered Micronized niclosamide in spray-dried lysozyme protein particles, Sum-10 um and greater in diameter at only 0.7%. This micronized protein spray dried niclosamide only provide low solubility niclosamide (1 uM) (light grey mucin) in the apparent aqueous 0.45% NaCl suspension it is delivered in and so does not optimally deliver niclosamide to the underlying epithelial cells. B) Administered niclosamide solution microdroplets. Microdroplets of niclosamide solution of 20 um-30 um at pH 8 (and up to 300 um at pH 9.2) from the sprayed solution land and spread on the air/mucin interface (darker grey mucin). 20 uM to 30 uM niclosamide solutions readily diffuse through and permeate the mucin to deliver niclosamide more optimally to the epithelial cells and their membranes including endosomes, mitochondria, and Golgi.

FIG. 32. Comparison between spray dried lysozyme-niclosamide and niclosamide solutions made here. The shaded boxes on the plot show the pHs of the solutions for: 1) the suspended micronized lysozyme spray dried niclosamide at pH 5.0-5.2 where its solubility is only ˜1 uM if in the low solubility monohydrate form, and 2) the pH 8.0-9.2 solutions of niclosamide that can have amounts in solution of 20 uM-300 uM.

### TABLES

TABLE 1. Supernatant concentrations of AK Sci Niclosamide (uM) measured by UV/VIS (UV5Nano cuvette, five measurements) for each measured supernatant solution pH, after stirring to equilibrium (48 hrs) at 20° C.

TABLE 2. Tabulated timeline of events comparing the: kinetics of mucosal epithelium, virus entry and replication, assembly and secretion, and the delivery of Niclosamide.

### DETAILED DESCRIPTION OF THE INVENTION

This patent application is written for a broad audience: the basic scientists involved in the physical chemistry and physical pharmacy of drug molecules; the pharmacology and cell biology researchers looking into drug-membrane interactions, intracellular cell pathways, efficacy and viability; the clinical researchers concerned about the COVID19 pandemic, new variants, and other respiratory viral infections; and the government agencies, foundations, infectious disease institutes, and companies world-wide who might consider taking on this niclosamide solution prophylactic and early treatment option of a niclosamide-based nasal and throat spray, and potentially considering it at cost or reduced profit, open source pharmaceuticals, starting generic.

It provides new data on how to formulate a notoriously low water-solubility drug, Niclosamide, more effectively as higher-concentration solutions by using only simple aqueous buffers. Data is presented on the underlying physical chemistry associated with the pH-dependent solubility of this weak acid and confirms that the amount of niclosamide in aqueous buffered solution does follow the Henderson Hasselbalch and precipitation pH models. For a particular commercially available material (Niclosamide from AK Sci, CA) the amount of niclosamide in aqueous solution at neutral pH 7 is measured to be, as expected, relatively low, at only a few micromolar. However, when dissolved in buffered solutions, niclosamide solution concentrations up to 704 uM can be achieved at pH 9.63. These simple solutions can now provide a pH 7.98 prophylactic nasal spray at solution concentrations of 20 μM that is 10× those expected for respiratory cell efficacy of ˜2 uM [34]. Moreover, the pH of this solution is safe, i.e., the necessary reduction of cell ATP is completely reversible, and airway epithelial cells do not die after drug washout [45]. For the throat spray, where higher pH is tolerated and a further compromise of cell viability is perhaps warranted, solutions of 300 μM can be made at pH 9.2, the pH of green tea. This principle of pH-dependent solubility is then used to demonstrate that niclosamide can be readily extracted from the regulatory approved Bayer's Yomesan tablets and other generics that are available world-wide.

Further experimentation and deeper analyses then show how such solutions can be formed, measuring dissolution rates as a function of pH. The data also show that care must be taken with this molecule and its supplier in formulation design, because, in aqueous media, certain supplied niclosamide, such as the one from Sigma, where any undissolved material or micronized niclosamide can readily revert to a very low solubility stable monohydrate [17] that would further compromise bioavailability of any microparticle or micronized material. Experiments also focus on precipitation from supersaturated solution to achieve pH-dependent morphologies, the aqueous dissolution of water-, ethanol- and acetone-co-solvates, and also evaluate a second commercially available niclosamide sample (from Sigma). Solubility and solution concentrations across the whole pH range are supported by optical images of crystal morphologies that show how the Sigma product already contains monohydrate like polymorphs that seem to promote the overnight conversion of stirred samples to the characteristic spiky monohydrate crystal polymorph, while the AKSci product only contains block-like crystals that do not convert so readily.

Guided by the pHp curve of the amount of niclosamide in stable solution across the whole pH range, a method of making is also introduced that involves dissolution of niclosamide in the absence of any powdered excess material by a solvent exchange technique. An ethanolic solution of niclosamide is injected into an excess of buffer solution (1:99 v/v ratio) to achieve the stable solution of niclosamide in buffer, with 1% ethanol.

In the Discussion section, all of this data is then evaluated in the context of providing a more optimized solution formulation of niclosamide for nasal and throat sprays, including the need to consider the actual solubility of the various polymorphs available as commercial supplies and subsequent conversions to the most stable monohydrate especially when left in equilibrium with excess undissolved material. Attention is turned to aspects of intranasal drug delivery in the context of epithelial tissue kinetics and viral transport, replication, and secretion of virions from the host cells, Here are considered: the biology of the epithelium-tissue target, its mucin barrier, and underlying epithelial cells; the timing of the viral lifecycle in its eclipse and latent periods that produce new secreted virions; and drug transport from solution through the mucin barrier to where niclosamide can end up, —in the cell's membranes and possibly in infected cell's lipid droplets. Each of these aspects have consequences for how prophylaxis and early treatment are made most effective. It is also pointed out that the timeline for the viral life cycle and the refresh rate of the mucosa generates a need for controlling the spread of virally infected cells and viruses in the mucus down the throat. i.e., given a viral life cycle that needs live host cells to replicate and takes 36 hrs, can this still occur when the top later of epithelial cells the virus infects are shed within 1-2 days after infection? Are these epithelial cells still functional and provide a safe harbor for the viral replication as the cells and secreted virus make their way in secretions down into the lungs to have their more devastating effects?

The above is all brought together in a final consideration of effective drug delivery systems for the naso and oropharynx. The main hypothesis to be tested is that a simple niclosamide solution, when used as prophylactic nasal and early treatment throat sprays could, at the very least, reduce the viral load that the vaccinated and unvaccinated immune system has to deal with. The work though generates a series of interesting sub-hypothesis to be tested regarding: quelling an over-reactive over immune response; could the virus (membrane) loaded with niclosamide be its own drug delivery system? Are infected cells self-selective for niclosamide because they contain more lipid droplets? Could niclosamide have a positive effect on slowing mucin production and movement, trapping the virus? Could misassembled non-competent virions act as their own vaccine? Are shed epithelial cells able to culture the virus during its replication cycle? These are offered to the community as potential proposals for investigation and to generate funding to further explore these important phenomena.

Finally, the application concludes with a brief discussion of what we still need to learn from preclinical animal testing, regulatory issues, and the most at-risk populations that could constitute much needed clinical testing and adoption of the prophylactic and early treatment nasal and throat sprays

Bottom Line: This patent application focuses on the point of viral entry, —the nasal epithelium. From there the virus particles and the cells that incubate them travel via secretion-transport into the throat and down into the lungs where they have their most devastating and sometimes fatal effect. It proposes a main hypothesis that a niclosamide solution as prophylactic-nasal and early treatment-throat sprays could reduce the viral load that the vaccinated and unvaccinated immune system has to deal with. It could thereby reduce the downstream complications that have been, and continue to be, so devastating, debilitating and even lethal especially now in unvaccinated populations. It could also limit asymptomatic and symptomatic spread of the more contagious variants due to break through infections now being seen in the vaccinated. Once established in this context, there is the potential for such niclosamide solution formulations to be developed, tested, and applied to other respiratory virus infections, since the action of this molecule is not on the particular virus or variant but on the host cells that they infect.

In exemplary embodiments, provided herein is a composition for application to a mucus membrane of a patient, comprising a buffered aqueous solution comprising about 10 uM to about 300 uM niclosamide, wherein the buffered aqueous solution has a pH between about pH 7.0 and about pH 9.2.

As described herein, it has been surprisingly found that a niclosamide composition can be prepared in which an amount of niclosamide of up to about 300 uM, can be maintained in solution in a buffered aqueous solution, so long as the solution has a pH of about 7.0 to about 9.2. In embodiments, the amount of niclosamide can be increased to about 700-800 uM, if a pH of about pH 9.5-9.7, i.e., about pH 9.6 As used herein, a “buffered aqueous solution” means a solution in which primary solvent is water that includes either a weak acid and its salt, or a weak base and its salt, which is resistant to changes in pH.

In embodiments, the buffered aqueous solutions (as intended for the nasal and oral-throat sprays) have a pH between about pH 8.0 and about pH 9.2, or between about pH 8.0 and about pH 9.0, or about pH 8.5 and about pH 9.0. Suitably the pH of the buffered aqueous solution is about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, or about pH 9.2.

Suitably, the amount of niclosamide in the niclosamide compositions described herein is between about 20 uM to about 40 uM, including about 25 uM to about 35 uM, or about 30 uM. In other embodiments, the amount of niclosamide can be 50 uM, 75 uM, 100 uM, 150 uM, 200 uM, 250 uM, 300 uM, 400 uM, 500 uM, 600 uM or as high as 700 uM, if the pH is raise about pH 9.0 to about pH 9.6 (e.g., about pH 9.1, about pH 9.2, about pH 9.3, about pH 9.4, about pH 9.5 or about pH 9.6.

Exemplary buffers that can be used in the compositions described herein are known in the art, and include for example, a Tris(hydroxymethyl)aminomethane buffer or a 4-(2-hydroxyethyl)-1-piperazineethanesulfonic acid (HEPES) buffer. Additional buffers such as phosphate buffered saline (PBS), Hank's Balanced Salt Solution (HBSS), Bis-Tris Buffer, Citrate buffer, Citrate-Phosphate buffer, MOPS buffer, PIPES buffer, potassium phosphate buffer, etc., can be used depending on the situation and pH required.

In embodiments, the compositions can further comprise additional excipients, including about 0.5% (v/v) to about 2% (v/v) carboxymethyl cellulose or other stabilizers. Additional excipients can include various antibacterial agents, thickeners, viscosity enhancers, etc.

In further embodiments, the compositions can further include about 1% (v/v) to about 2% (v/v) ethanol. As described herein, methods of preparing the niclosamide compositions can include the use of a solvent injection technique, wherein a solution of niclosamide (suitably in ethanol) is injected into an buffered aqueous solution. Niclosamide can also be prepared in dimethylacetamide and acetone, for example.

As described herein, the compositions are suitably formulated as a nasal spray, an oral-throat spray, a nasal rinse, or an oral-throat rinse.

In additional embodiments, provided herein is a composition for application to a mucus membrane of a person, consisting of or consisting essentially of a buffered aqueous solution comprising about 10 uM to about 300 uM niclosamide, wherein the buffered aqueous solution has a pH between about pH 7.0 and about pH 9.2. In embodiments in which the composition consists essentially of the recited components, specific components such as additional lipid or protein stabilizers are specifically excluded. In other embodiments, specific lipid or protein stabilizers can be included, as called out in the description provided herein.

Also provided herein is a method of reducing a viral infection comprising administering a composition comprising a buffered aqueous solution comprising about 10 uM to about 300 uM niclosamide to a mucus membrane of a person, wherein the buffered aqueous solution has a pH between 7.0 and pH 9.2.

Suitably, the composition is administered to a nasal passage of the person, or can be administered to the patient's throat. If administered to the nasal passage, it is suitably administered as a nasal spray or a nasal rinse. If administered to the throat, it is suitably administered as an oral-throat spray or as an oral rinse.

The methods of prevention and treatment described herein are suitably used for the prevention and treatment of a coronavirus infection, including a coronavirus variant respiratory virus infection. In exemplary embodiments, the coronavirus viral infection is from 229E (alpha coronavirus), NL63 (alpha coronavirus), OC43 (beta coronavirus), HKU1 (beta coronavirus), MERS-CoV (Middle East Respiratory Syndrome (MERS)), SARS-CoV (severe acute respiratory syndrome, (SARS)) or SARS-CoV-2 (coronavirus disease 2019 (COVID-19)). Additional respiratory viruses, such as influenza, can also be treated or prevented by the methods described herein.

As described herein, in embodiments, replication of viral particles of the viral infection is reduced by at least 50%, compared to an untreated viral infection. In embodiments, the viral infection is reduced by at least 50%, at least 60%, at least 70%, at least 80%, at least 90%, or completely reduced (i.e., 100%). Measurement of replication of viral particles can be carried out using various methods known in the art to measure viral replication in either a cellular, in vitro assay, or in an in vivo assay in an animal patient, including a human. In embodiments, the replication of viral particles is reduced within about 4 hours, more suitably within about 2 hours or within about 1 hour following the administration, including within about 30 minutes.

Also provided herein is a method of preparing a niclosamide composition, comprising injecting an ethanolic niclosamide solution into a buffered aqueous solution to produce the niclosamide composition in the buffered solution. As described herein, this injection method suitably includes dissolving the niclosamide in ethanol (or other suitable solvent) and then injecting the niclosamide into a buffered aqueous solution, with stirring, so that the niclosamide becomes dissolved in the buffered aqueous solution. In such methods, the resulting composition will maintain a small amount, e.g., about 1% to about 2% v/v of ethanol, or even smaller amounts, i.e., 0.1%-1% v/v of ethanol, depending on the starting concentration of niclosamide.

In exemplary embodiments, the ethanolic niclosamide solution is prepared at 100 uL of a 2 mM niclosamide and is injected into 10 mL of a pH 9.2 buffered aqueous solution to make a 20 uM solution of niclosamide by diluting the initial ethanolic niclosamide in a 1:99 v/v ratio. Other preparation include, for example, 10 mL of a 2 mM niclosamide and is injected into 1 L of a pH 9.2 buffered aqueous solution to make 1 L of a 20 uM solution of niclosamide by diluting the initial ethanolic niclosamide in a 1:99 v/v ratio; 100 uL of a 30 mM niclosamide and is injected into 10 mL of a pH 9.2 buffered aqueous solution to make a 300 uM solution of niclosamide by diluting the initial ethanolic niclosamide in a 1:99 v/v ratio; 10 mL of a 30 mM niclosamide and is injected into 1 L of a pH 9.2 buffered aqueous solution to make 1 L of a 300 uM solution of niclosamide by diluting the initial ethanolic niclosamide in a 1:99 v/v ratio.

As described herein, the niclosamide compositions are suitably prepared under sterile conditions, filled into bottles (e.g. sterile vials are capped and sealed, or squeeze bottles or rinse bottles are prepared and sealed), capped and sealed, so as to be provided to people.

In still further embodiments, provided herein is a method for preparing a niclosamide composition, comprising crushing a niclosamide tablet into a powder and incubating them in a buffered aqueous solution to extract niclosamide. Suitably, the niclosamide tablets are YOMESAN® tablets, which contain 500 mg niclosamide, for the treatment of infestation of various tapeworms. The excipients are: maize starch, talcum, sodium lauryl sulphate, povidone, vanillin, magnesium stearate, saccharin sodium.

As described herein, suitably the buffered aqueous solution has a pH of pH 7 to pH 9.5. In embodiments, the amount of niclosamide table is 5 mg and the amount of buffered aqueous solution is 10 mL. Suitably, the niclosamide composition is further filtered through a 0.22 um filter, and in embodiments, the resulting composition is a buffered aqueous solution with a pH 9.34 and the final concentration of extracted niclosamide is 348 uM.

### 1.2 Why Niclosamide?

### 1.2.1. Niclosamide has Broad Clinical Applications in a Number of Diseases and Conditions

Over the past several years the anti-helminthic drug niclosamide has emerged as a quite unusual candidate that may have broad clinical applications for the treatment of many diseases other than just those caused by gut-parasites. Many current anti-virals either attempt to disrupt the synthesis and assembly of viral proteins (viral proteases, RNA-dependent RNA-polymerase, virus helicases, viral spike and structural proteins) or target host proteins and mechanisms required by the viral replication cycle (including, boosting interferon response, ACE2 receptors, cell surface and endosomal proteases, and clathrin mediated endocytosis) [46]. As discussed below (1.2.3), Niclosamide offers a different and potentially very effective way to combat viral infection because it enters host cell membranes as a lipophilic anion where it acts as a proton shut, dissipating pH gradients across a range of host cellular-organelle membranes, including mitochondria, endosomes, and lysosomes, and even the Golgi. In an excellent and comprehensive review, Chen and Mook et al [47] reported that niclosamide when tested, mainly in cells and a few preclinical animal cancer models, has efficacy that includes cancer, bacterial and viral infection, metabolic diseases such as Type II diabetes, NASH and NAFLD, artery constriction, endometriosis, neuropathic pain, rheumatoid arthritis, sclerodermatous graft-versus-host disease, and systemic sclerosis. There are also reports of activity in Parkinson's [48] and COPD [19, 27].

As a drug, Niclosamide is relatively unique in that it acts upstream of almost all cellular pathways as an inhibitor of oxidative phosphorylation and so reduces the availability of ATP in the cell. It is therefore hardly surprising that, pharmacologically, niclosamide has been shown to be a very “dirty drug”. For example, in cancer cells, it inhibits (at least) 17 different pathways [49, 50], including Wnt/β-catenin, mTORC1, STAT3, NF-κB, aromatase, and Notch signaling. Especially in cancer there appears to be additional mechanisms associated with mitochondrial fragmentation that induce cancer cells to go into Apoptosis [51]. However, niclosamide is relatively non-toxic to healthy cells [52], i.e., “niclosamide is only toxic if the cell makes it toxic”. Niclosamide also induces cell cycle arrest in G1 phase in head and neck Squamous cell carcinoma through tumor suppressor mechanisms [53]. In our and (other's) recent studies, there are no S phase cells after incubation with only micromolar niclosamide. As an adjunct to chemotherapies, niclosamide also has antineoplastic effects through direct STAT3 inhibition [54], where its inhibition of cell proliferation enhances the responsiveness of esophageal cancer cells to chemotherapeutic agents.

In the area of Osteosarcoma (OS), that we have been particularly interested in [13], niclosamide inhibits multiple pathways that promote survival and growth that are known to be dysregulated in OS, including, again, the Wnt/β-catenin, Akt/mTOR/PI3K, JAK/STAT, NOTCH, and NF-κB pathways [49, 55-58]. As a result, our re-formulation of niclosamide as a Niclosamide Stearate Prodrug Therapeutic (NSPT) showed activity in inhibiting metastatic spread to the lungs of mice in a one-day-chase osteosarcoma mouse model of metastatic lung disease [13]. The NSPTs have also slowed and eliminated the appearance of Osteosarcoma lung metastases in a canine feasibility trial for 6 of 10 dogs, including out to almost 3 years post treatment for three of the dogs [59].

Bottom Line: The reason for niclosamide's broad range of influence is its action at one of the highest levels in the cell; as an uncoupler of oxidative phosphorylation in mitochondria. Hence, a reduction or absence of ATP grinds many of the down-stream processes to a halt. This now extends to its influence on viral infection at the single cell host level, as introduced and reviewed next.

### 1.2.2. Niclosamide Also has Broad Virostatic Activity as a Host Cell Modulator

While vaccines and antibody treatments are certainly effective, they are designed to work only after infection has taken place, i.e., they do not prevent infection. They are also limited to a particular virus, a particular viral protein, eventually need to be boosted, or redesigned for an emerging viral variant. In contrast, Niclosamide is a host cell modulator and so is specific for every virally infected or infectable cell. As postulated by Laise et al [46], “Targeting host cell mechanisms may have more universal and longer-term value because the same host actors may be required by multiple, potentially unrelated viral species and because host target proteins mutate far less rapidly than viral proteins, thereby limiting emergence of drug resistance”. We concur. The action of niclosamide is more virostatic (than directly anti-viral) in the sense that it controls replication of any virus and any new variant [7] that uses the cell's own machinery to replicate. As discussed below (1.2.3), it also inhibits entry of viral RNA into the cell and, if translated, can also interfere with final viral assembly in the Golgi such that the cell secretes non-competent virions. Thus, niclosamide's mechanism of action is not directly on the viral RNA, or on blocking transcription/translation enzymes, or inhibiting the receptor-ligand interactions that first bind the virus to the cells. Rather, its targets are the intracellular membranes that otherwise facilitate these processes and where it dissipates their pH gradients reducing or eliminating function.

Since the early 2000's niclosamide has shown broad spectrum virostatic activity against various viral infections with nanomolar to micromolar potency [14, 32, 34, 41, 43]. These have included SARS-CoV, MERS-CoV, ZIKV, HCV, and human adenovirus. With respect to SARS-COV and SARS-COV-2, in March 2020 Jeon et al [14] at the Korean Pasteur Institute measured an IC₅₀ for viral replication of 0.28 uM in Vero 6 cells and followed this with a second screen (by Ko et al, [34]) in the more relevant, although still not ideal, Calu-3 cells (human lung epithelial isolated from human lung adenocarcinoma) of 0.84 uM. They also showed a complete IC₁₀₀ inhibitory activity for viral replication at the level of 1 uM in Vero 6 cells and an estimated 2 μM-3 μM in the Calu-3 cells [34]. These results were recently confirmed by Braga et al [18] for SARS-COV-2 also in Vero 6 cells with an IC₅₀ for viral replication of 0.34 uM, as well as in respiratory Calu-3 cells where 2.5 uM niclosamide showed inhibition of viral replication. Similarly, Mostafa et al [29], also in Vero E6 cells, showed that, while the IC₅₀ for inhibiting viral replication was actually even lower, at 0.16 uM niclosamide, the half maximal cytotoxic concentration for the host cells (CC₅₀) was 204.6 uM, representing a therapeutic (selectivity) index of 1279x. Mirabelli et al [60] also measured an IC₅₀ of 0.14 uM for niclosamide in infected liver Huh-7 cells. Finally, Zhu et al [61] showed that SARS-COV-2 causes multinucleated syncytial cells arranged in a net-like structure observed in plaque regions. Here, the data from Braga et al showed that niclosamide protected against syncytia in vitro [18] where cells still infected in the presence of niclosamide were no longer syncytial. Incidentally, Ko et al, [34] identified the protease Nafamostat as a more potent antiviral drug candidate, with activity in the few nanomolar, and so this could be another drug that is prime for formulation.

Bottom Line: Multiple papers in the literature have now identified niclosamide in several studies and different cell lines as a potentially viable treatment option for SARS-COV-2 and other viral infections [40-44]. Most studies are in Vero 6 cells, although the Korean Pasteur group have extended their screens to human lung Calu-3 cells [34]. What they found was that the IC₅₀ anti-viral activity for inhibiting replication was slightly less potent in the Calu-3 cells by about a factor of 3 and (reading from their graph) an estimated 2 uM to 3 uM for complete inhibition of viral replication. As discussed in detail below (2.2), niclosamide only has a water solubility of ˜1 uM to 2 uM at nasal pH and so, while still not primary lung airway cells, with an IC₁₀₀, of 2 uM to 3 uM to inhibit replication in the host cells, this data points to the need for much higher concentrations of niclosamide in solution than can be obtained from just niclosamide solubility in water alone or by the dissolution of particulate or micronized niclosamide.

### 1.2.3. Niclosamide Inhibits Three of the Six Stages of Viral Infection

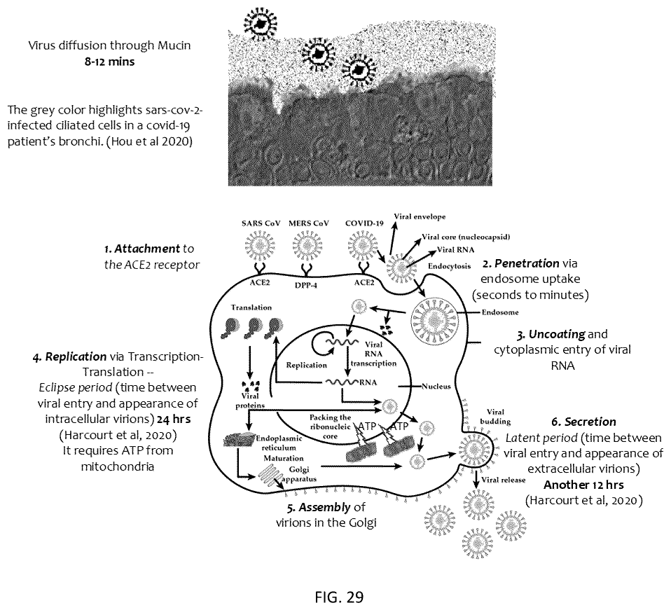

Looking deeper into the mechanisms of niclosamide activity, specifically with respect to viral infection, as discussed by Goulding [4], because viruses are obligate intracellular pathogens, they cannot replicate without utilizing the machinery and metabolism of a host cell. As shown later in FIG. 29 and discussed additionally, there are six basic stages that are essential for viral replication: attachment, penetration, uncoating, replication, assembly, and virion release. Niclosamide can inhibit three of these:

1. Uncoating: Because it is a lipophilic anion, niclosamide can partition into lipid membranes, and can shunt protons out of the endosome. Niclosamide can therefore reverse the usual acidification of endosomes that the virus needs for the conformational change required by the spike protein to fuse with the endosome membrane. As described by Fehr and Perlman for coronaviruses [62] and confirmed for SARS-COV-2 [63, 64], the lower pH is required to expose a fusion peptide that inserts into the membrane and allows for the mixing of viral and cellular membranes, resulting in fusion and ultimately release of the viral genome into the cytoplasm. Niclosamide has therefore been shown to prevent viral fusion, uncoating, and the entry of viral RNA in cells infected with influenza [22], the Dengue virus, [65] and this has now been shown to be the case for SARS-COV-2 [30]. Also, associated with its ability to dissipate other pH gradients in the endosomes and lysozomes and acidify the cytoplasm, niclosamide is an autophagy-inducing compound [41], which adds to its potential as a treatment against SARS-CoV-2.

2. Replication: By dissipating the pH gradient across the mitochondrial inner membrane [39], niclosamide inhibits oxidative phosphorylation and so reduces available energy for a virus to replicate in the host cell [47]. Thus, niclosamide also acts as a proton shunt in the inner membrane of mitochondria and dissipates the normal pH gradient, thereby inhibiting the transport of H⁺ ions through the transmembrane ATP synthesis enzyme that drives ATP production. As a result, ADP does not get translated to ATP, and there is a reduction in cell viability (but not necessarily death for normal cells, as Kim at al have recently shown, [45]). Thus, niclosamide effectively slows down the metabolism of the infected cell, and the extent to which is does this can be titrated with niclosamide dose. The consequences of even partially reducing ATP in a potentially infectable or, indeed, infected host cell, is that the virus cannot use the cell's own machinery to replicate itself [14, 41]. In vaccinia poxvirus, virus production actually requires increased amounts of ATP [66, 67], and so, if this holds for coronavirus, niclosamide could provide an even greater inhibitory effect. Specifically for SARS-COV-2, niclosamide shows prophylactic inhibition of SARS-COV-2 replication [14], such that, when niclosamide was already present in Vero 6 cells, SARS-COV-2 could not replicate. Of 47 antiviral drug candidates tested against SARS-CoV-2 by Jeon et al [14], niclosamide completely inhibited viral replication at an IC₁₀₀ of 1 uM in Vero 6 and 2 μM to 3 μM in Calu-3 [34]), while host cell viability extended to >100 uM. Interestingly, in the Vero 6 cells, the IC₅₀ for niclosamide of 0.28 uM was 30 to 40 times more effective than chloroquine, lopinavir, and remdesivir, with IC₅₀ values of 9.12 uM, 7.28 uM, and 11.41 uM respectively [14].

3. Assembly: Niclosamide also inhibits the manufacture of viral protein assembly in the pH-dependent activity of the Golgi [65]. It prevents E glycoprotein conformational changes on the flavivirus virion surface resulting in the release of non-infectious immature virus particles with un-cleaved pr-peptide from host cells. And so again, while not yet tested, if this holds for SARS-COV2 niclosamide spray could have additional effects that limit viral load and infection. Interestingly such non-competent virions may hypothetically act as their own “vaccine”. This hypothesis is still to be tested.

Bottom Line: Collectively, all these literature studies support the potential application of niclosamide as an antiviral agent, with a virostatic mechanism against many viral infections, including the SARS-COV-2 of current interest, that could extend to its more contagious variants [68], and also the perennial influenza and many others [32]. They highlight a series of unique mechanisms of action of the drug, i.e., niclosamide can partition into lipid bilayer membranes as a lipophilic anion. Thus, all of niclosamide's multiple different pathways in cancer [50] and now infected host cells [14, 32, 34, 41, 43] are based on lipophilic proton shunt activity that dissipate pH gradients in endosomes and lysozomes, the mitochondrial inner membrane, and in the Golgi. Importantly, these actions inhibit viral entry and viral replication, and promote the mis-assembly of non-infective virions. Niclosamide has therefore emerged as a very interesting and potentially successful ubiquitous anti-viral virostatic that could be used in conjunction with all other treatments of COVID19 and other viral infections as a prophylactic preventing viral replication or early treatment option that reduces viral replication in the nose and throat.

### 1.2.4. The Need for New Formulations

Given that the in vitro cell data is overwhelming for its virostatic potential, the challenge we have met is to optimize the formulation for this typically low-solubility drug by inventing a solution of Niclosamide [16] and obtaining concentrations of 20 μM at pH 8.0, 300 uM at pH 9.2, and up to ˜700 μM at pH 9.63. These solution concentrations are well above its in vitro inhibition of viral replication in Vero 6 and Calu-3 cells of 1 uM-3 uM [14, 32, 34, 41]. Also, as mentioned above and discussed in more detail in section 5. Discussion, preliminary studies, by Kim [45] in airway epithelial cells show that, with an IC₅₀ of 20 uM to 30 uM niclosamide, these necessary reductions in cell viability and ATP are reversible and do not result in any cell death. Reformulated and tested as a nasal and throat spray, niclosamide could represent an effective prophylactic and early treatment for many viral infections, including ones that mutate and escape current vaccine protection. Thus, a prophylactic nasal and an early treatment throat spray that “puts the virus in lockdown” or does not even let it into the nasal epithelial cells, would be a huge boost in the control of viral infection and spread for unvaccinated populations and also vaccinated spreaders where the vaccine is highly protective against hospitalization and severe disease but is less so for asymptomatic or break through infections, that seem to be linked to the delta variant [69, 70].

Searching for COVID19 Niclosamide at clinicaltrials.gov [35] and at COVID19 help.org [71], shows that there are fourteen studies that are currently recruiting or not yet recruiting; also a few preclinical studies deserve some mention.

Oral Administration: Seven trials are on-going simply administering the conventional niclosamide (Bayer-Yomesan) oral tablets as used in the original anti-helminthic application and now repurposed for COVID19. Of these, there are only two in the USA. One additional oral trial is by the University Hospital, Lille, looking to prevent hospitalization or death, by administering Hydroxychloroquine or Diltiazem-Niclosamide for the Treatment of COVID-19, again orally. While certainly worth trying, especially for gut infection, as shown by a recent pancreatic cancer trial by Schweizer et al [31], using the same tablets, systemic delivery is poor—plasma concentrations were only 35.7-182 ng/ml (0.1 uM-0.6 uM) when 500 mgs was given 3 times per day (1.5 g) for four weeks. This trial (for prostate cancer) was closed for futility. And so, oral tablets in the COVID19 trials may be sub-optimal for systemic viremic disease and as any form of prophylactic or early treatment regimen. To address this poor response to oral administration of Yomesan, a new Phase 2 trial by AzurRx BioPharma is currently testing micronized niclosamide oral “immediate release” tablets for COVID-19 Gastrointestinal Infections [72]. It is hoped that such “micronization will allow superior dissolution in gut fluids” and that this in turn “may allow local niclosamide concentrations to reach anti-viral levels”. This kind of micronized niclosamide is limited by intrinsic solubility, not speed of dissolution, that, as shown in section 4. Results, a Yomesan tablet starts to dissolve immediately and in pH 8.6 to 9.4 buffer took ˜30 minutes to reach saturation under well stirred conditions. It is unlikely that the micronized “immediate release” tablets will provide more niclosamide in solution in the gut than Yomesan tablets. As is well known [73], the intraluminal pH changes from highly acid in the stomach to ˜pH 6 in the duodenum, then gradually increases in the small intestine from pH 6 to about pH 7.4 in the terminal ileum, then drops to 5.7 in the caecum, and again gradually increases, reaching pH 6.7 in the rectum. Since, niclosamide solubility is determined by the nature of the dissolving and equilibrating polymorph, as shown in this patent application, the amount of the niclosamide monohydrate in solution for any of these pHs is still only on the order of 1 uM and the virus replication appears to be in the 1-3 uM level [14, 32, 34, 41].

In preclinical development CNPharm have developed a novel organic-inorganic hybrid oral formulation, Niclosamide-dehydrotalcite CP-COV03, (a niclosamide-clay intercalate coated with nonionic Tween polymer surfactant). In in preclinical studies CP-COV03 has apparently shown 12.5× improved (12 hour) bioavailability and the half-life compared to existing Niclosamide [74]. As just announced in May 2021 [75], Hyundai Bioscience plan to proceed to clinical trial with CP-COV03. A concern here is that the tested dose in rats was 50 mg/kg Niclosamide, which converts to a Human Equivalent Dose (divide by 6.2) [76], of 8.06 mg/kg and for a 70 kg human that would be 565 mg per dose. They suggest taking it twice per day, every day, and at these kinds of doses, while Yomesan is safe systemically, it has shown Grade 1 nausea, anorexia and weight loss [31], and if higher doses are deemed necessary in clinical trials these toxicities could be dose limiting as debilitating nausea, vomiting, diarrhea and colitis. The drug could therefore face non-compliance and adverse events issues and long-term prophylactic use could be questionable.

Intramuscular Injection: Four trials are for Daewoong's intramuscular injectable niclosamide including one at Tufts [36]. Preclinically there are efforts being made by Hobson et al [77] developing a nanoprecipitation and spray drying process to form niclosamide (NCL) in an aqueous solution of stabilizers and sugars nano-dispersions for long-acting injectable delivery. Plasma concentrations though drop rapidly within a day to 100 ng/mL, levels below anti-viral efficacy, and this could also face compliance issues of having to inject daily.

Unknown route: One, Imuneks Farma, in Turkey, is administered as 200 mg niclosamide in 10 mL of suspension to 200 patients, but the route of administration is not disclosed.

Nasal Inhalation route. Although currently there are no combined nasal-throat formulations being clinically tested as proposed here, one clinical trial for nasal spray and inhalation, is starting. Union Therapeutics Denmark, is now starting initial clinical trials with a micronized niclosamide in spray dried lysozyme supplied by TFF Pharmaceuticals [78] in at-risk kidney patients at Addenbrookes, UK [37]. In October 2020, this particulate niclosamide nasal spray has already been shown safe in 44 heathy volunteers [79]. While safety is not efficacy, this can only bode well for our optimized niclosamide solution. This successful development and positive initial safety testing of an inhalable, spray-dried, microparticulate niclosamide, motivates further development of niclosamide in perhaps more optimized mucin-penetrating, and more easily produced solution formulations for nasal and throat spray applications. A more detailed analysis of this spray-dried, microparticulate niclosamide is provided at the end of the discussion section, —5.4 A Cautionary Tale for any microparticulate material that is used to target the mucosa. Given the data obtained in this paper, there is some concern, again, over the amount of niclosamide that can be delivered by such a microparticle form of niclosamide embedded in microparticles of spray dried lysozome. The protective nasal mucin excludes any particles bigger than a few hundred nanometers and so would be expected to exclude 5-10 μm spray dried lysozyme and micronized niclosamide.

Bottom line: With a raging world-wide pandemic and more contagious variants already in circulation, and limited vaccination world-wide, Niclosamide, is a broad spectrum virostatic host cell modulator [32] that has already shown convincing activity as a virostatic against SARS-COV-2 [40-44]. When formulated as a simple mucin-penetrating solution, niclosamide can potentially help to control initial infection and viral loads across the board, while vaccines and other oral antivirals provide subsequent immune protection. As with vaccine development, there is a need for millions if not billions of doses, (especially on a frequent basis) and so there is certainly room for several prophylactic formulations, products, and companies that can help to control such initial viral infection and early symptoms. A low dose (20 uM) prophylactic solution of niclosamide at a nasally safe and acceptable pH of 8.0 and a (up to 300 uM) throat spray at pH 9.2 would be one of the simplest and potentially most effective sprays from both an efficacy standpoint as well as manufacturing and distribution, with no cold chain.

### 2. Preformulation Drug Characterization (pKₐ, S_{w}, Log P)

This section provides the theoretical and rudimentary basis that lay the foundation for the niclosamide solution formulation. It is essentially “first year pharmacy” applied to niclosamide. It provides an understanding of the basic molecular properties of the compound, such as its pKa, the resulting pH-dependent aqueous solubility (S_{w}), and its octanol water partition coefficients (Log P and Log D). ach of these properties then go into planning and interpreting data from experimental determination of intrinsic solubility of the low solubility acid, amounts in solution, precipitation pH, and dissolution characteristics. They also highlight the differences and therefore need to evaluate these properties for different morphological forms, from different niclosamide suppliers, as well as extracted from commercially available Niclosamide tablets, e.g., Bayer's Yomesan.

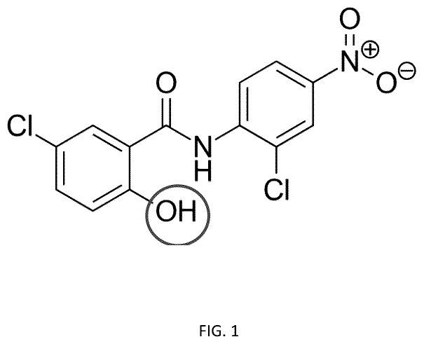

As shown in FIG. 1, Niclosamide (5-Chloro-N-(2-chloro-4-nitrophenyl)-2-hydroxybenzamide), is a chlorinated salicylanilide, that contains a zwitterionic nitro group and an ionizable phenolic OH. The most important moiety for the current application is the ionizable phenolic OH. Analysis starts with the effect of deprotonation of Niclosamide's salicylic OH to give the negatively charged salt (termed Nic₋ᵥₑ), which occurs over a certain range of pH and is determined by the molecule's pKa. The reason this is important is that the protonated acid, which is the prevalent species at neutral pH 7 and lower, is expected to have a much lower solubility than the charged salt, and so by precisely identifying the pKa, the solubility can be determined and increased at higher pH. It is this weak acid pKa that enables the new higher concentrations of niclosamide to be prepared in simple buffer solutions. The questions answered in this study then are: “How much does the solubility increase with increasing pH?” “How high does the pH have to be to achieve niclosamide concentrations that are well in excess of the anti-viral therapeutic levels of 1 μM-3 μM?” and, “Are these levels expected to be safe in the nasal and buccal/throat epithelium?

Thus, the parameters we are most concerned about are the pKa, that determines the amount of each species in solution that is available at each pH, (i.e., the protonation-deprotonation balance), niclosamide's intrinsic aqueous solubility (Sₒ) and the Log P, (or Log D—the pH dependence of this partition) which underlies the ability of niclosamide to partition into the various lipid bilayer membranes and exert its proton shunt effect. Taking these in turn provides rudimentary and important quantitation of the properties of this drug that are the basis for the new and simple solution formulation and developed spray, its mechanisms of action, and that should really be considered in any other nano and especially microparticle formulation of niclosamide.

### 2.1. pKₐ

The pKa is defined as the negative base-10 logarithm of the acid dissociation constant (Ka) of a solution). It is an important parameter to consider here because pKa and pH are equal when half of the acid has dissociated. Although experimentally challenging for such a low solubility molecule as niclosamide, the pKa can be measured experimentally [80] or calculated theoretically [81]; both are available in the literature, and a range of values are reported for niclosamide.
Jurgeit et al in their paper, “Niclosamide is a Proton Carrier and Targets Acidic Endosomes with Broad Antiviral Effects [22] report that Niclosamide has an estimated pKa of 5.60 as given in [82], calculated using solubility data.
This pKa value of 5.60 is also confirmed at U.S. EPA ECOTOX database [83] related to Pesticide Risk Assessment.
At Drugbank.ca, it is quoted as 6.89 and references calculations by Chem Axon.
Similarly, at ACD/iLabs, it is calculated as having a pKₐ of 7.2±0.4.
In [84] they say, Niclosamide has a very low pH-dependent water solubility due to the phenolic moiety with pKa=6.38 [85].
   Thus, the literature has a range of reported pKₐ values from 5.6 to 7.2, and the average of all these values is 6.52.
   Once the pKa is measured (or estimated) the well-known Henderson-Hasselbalch equation calculates the fractions of each species. For a weak acid, like niclosamide, this is simply:

pH=pKₐ+Log₁₀[Nic₋ᵥₑ]/[Nic_{OH}] Eqn 1

This equation can be represented as:

pH=pKₐ+log(α/1−α) Eqn 2

where α is the fraction ionized species as the deprotonated salt, Nic₋ᵥₑ.

As outlined in [86] the fraction ionized can be obtained as:

α=1/(1+10^{(pKa-pH)}) Eqn 3

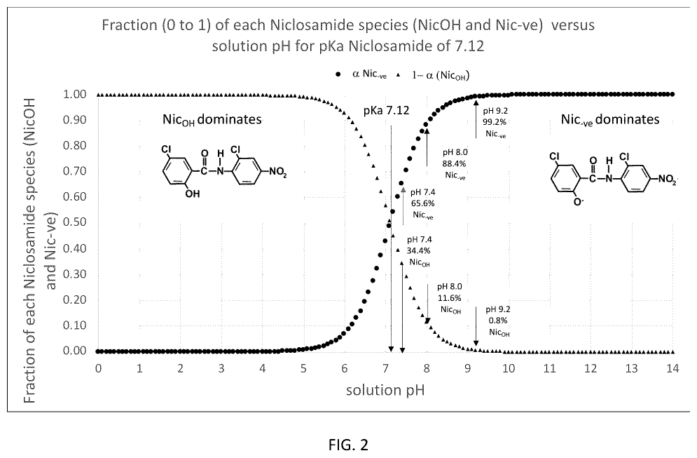

As shown in FIG. 2, for a known pKa, the Henderson-Hasselbalch equation allows a plot to be made of the expected fractions of each species: α is Nic₋ᵥₑ (filled circles) and 1−α is Nic_{OH}, (filled triangles) as shown in this plot, exemplified for pKa of 6.87 (derived from experiment, see later, Results). As can be seen, at low pH the dominant species is the protonated acid Nic_{OH}. Then, with increasing pH, Nic_{OH} deprotonates and the salt Nic₋ᵥₑ starts to become more prevalent.

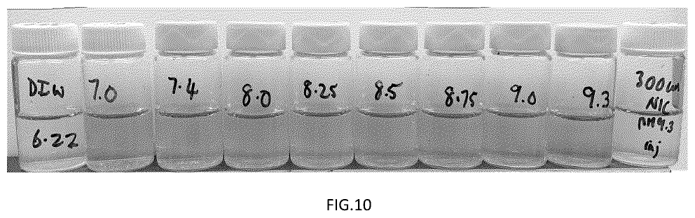

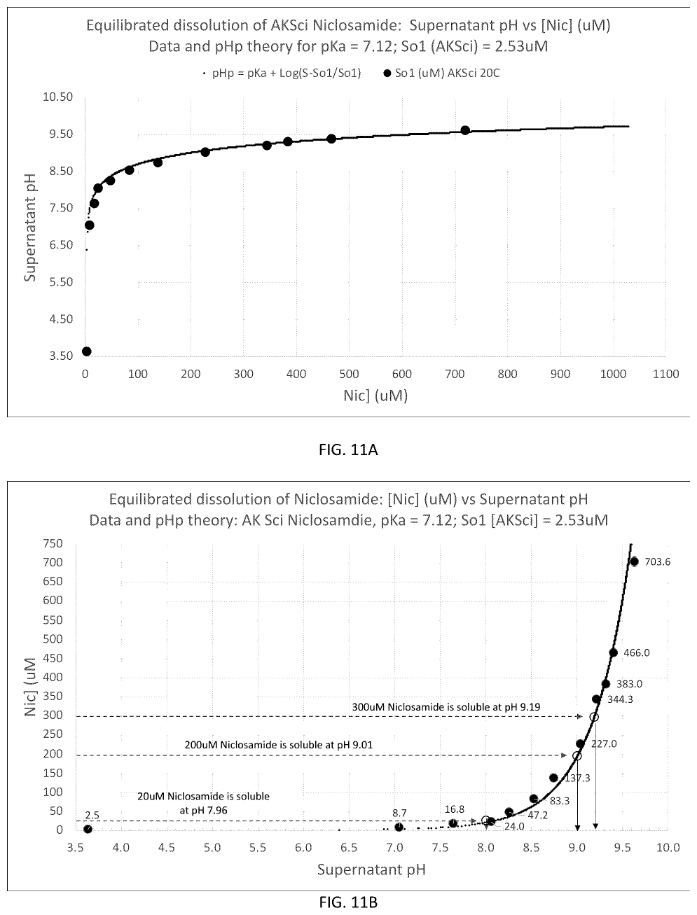

As seen later in FIG. 11, fitting the pHp data for the intrinsic solubility Sₒ measured for the protonated acid of 2.53 uM, AKSci niclosamide gave a pKₐ of 7.12, and so this, in and of itself, is a way to “measure” pKa. Thus, if this value is used as an example, there is 0.5 of each niclosamide species at pH 7.12. As can be seen in FIG. 2, at low PH. (less than pH 4), the low solubility acid represents 100% of niclosamide in solution, and the fractional amount of this species is expected to decrease with increasing pH.

Similarly, the fraction of the more soluble charged salt is expected to increase with increasing pH; it becomes the 100% dominant species at ˜pH 10. Thus, a solution equilibrium is expected between the acid and the salt for any solution pH between pH 4 and 10. For example, at physiological pH of 7.4, the acid and salt are 34.4% and 65.6% of the niclosamide in solution, respectively. At our target nasal spray concentration of 20 uM at pH of 8 the amounts of acid and salt are 11.6% and 88.4%; and at pH 9.2 (our target 300 uM niclosamide for the oral-throat spray) Nic-ve is 99.2% of the acid salt pair and the low solubility acid is only 0.8%. It is the high solubility salt that does the proton shunting across membranes.

The key result from this graph for the new application of niclosamide as a nasal and throat spray solution, is that the amount of the low solubility Nic_{OH} is expected to decrease and the amount of the higher solubility Nic₋ᵥₑ, is expected to increase with increasing pH. Overall, then, the amount of niclosamide in aqueous buffered solution is therefore expected to be higher at higher pH. This is the basis for the current solution formulation of niclosamide. As described later, we are clearly limited as to the range of pH that can be applied in the nasal pharynx, but less so, in the oral pharynx. However, at first glance, a pKa of 7.12 does appear to give a workable range and a potentially useful formulation for a niclosamide solution.

### 2.2. Aqueous Intrinsic Solubility (Sₒ)

The next challenge is to obtain the maximum concentrations of niclosamide in solution as a function of pH. For this we need to measure or estimate the solubilities of each species, or at least one of them, the low solubility acid. Obviously, the reason solubility is important is because it determines the amount of bioavailable drug that can be sprayed as well as the concentration of drug that gets through the mucin to the all-important layer of epithelial cells. As described above, if niclosamide is delivered as relatively low solubility micronized material, there is less chance of molecular water-soluble niclosamide reaching the epithelia cells to act as a prophylactic or early treatment of the virus in the epithelial cells. It is here that we have focused on what it takes to provide a mucus-penetrating niclosamide solution rather than micronized material simply trapped at the mucus surface.

Values of aqueous solubility of niclosamide in the literature are again widely distributed. One reason may be the different polymorphic forms that are tested as well as pH (that is often not specified). As quoted in the Graebing paper [87]) the solubility of niclosamide in water is given as 5-8 mg/L (14) which is 15.5 uM to 24.5 uM. An EPA document says: “Niclosamide is practically insoluble in water” and gives a value of 1.05×10⁵ g/100 mL which equals 0.321 uM. As characterized for niclosamide in a series of papers by de Villiers et al [5, 17, 88, 89], polymorphism, hydrate versus anhydrate, and cosolvates can change the solubility, dissolution, and chemical stability of the drug compound. In what is probably the most reliable set of values, van Tonder et al [17] the solubility of the crystal forms of niclosamide were measured in distilled water and found to decrease in the order: anhydrate [symbol] monohydrate HA>monohydrate HB, with the HB monohydrate being the most stable form. The values reported were: Anhydrate 13.32±3.18 mg/mL; Monohydrates HA 0.95±0.06 mg/mL; Monohydrates HB 0.61±0.09 mg/mL. These values convert to 40.4 uM, 2.9 uM and 1.87 uM, respectively. As described in the Experimental section in Methods and Results, it has been possible to obtain some estimate of the limiting solubility of niclosamide from a UV/VIS by painstakingly evaluating two noisy scans, i.e., one for the blank buffer and one for the sample at pH 3.66 where the niclosamide solubility is expected to be its absolute lowest. As presented later, this solubility for the AKSci niclosamide is only 2.53 uM±0.1 uM, and the value for the presumed monohydrate (from equilibrated Sigma niclosamide) is even less at 1.01 uM±0.26 uM, and so are actually in good agreement with this literature. Even though the pH was not measured or reported in van Tonder's paper, a DI water solution of niclosamide has a pH of ˜6.5, (as measured here) and the solubility obtained here for the presumed monohydrate at pH 7.09 of 1.24 uM±0.23 uM, is in good agreement with van Tonder.

Lastly, there appears to be only one mention of pH dependent solubility of Niclosamide in the literature. The Pesticide Handbook [90] reports that the solubility of Niclosamide (polymorph not specified) at pH 6.4 as 1.6 mg/L (which is 4.89 μM) and at pH 9.1 as 110 mg/L (which is 336.3 μM), suggesting that the solubility of niclosamide can vary by at least a factor of almost 100× depending on pH between 6.4 and 9.1. This data supports the new findings here across the pH range. In any event, care must be taken to specify pH and the polymorph when reporting any solubilities or amounts in solution for niclosamide.

### 2.3. Amounts in Aqueous Solution and the Precipitation pH (pHp)

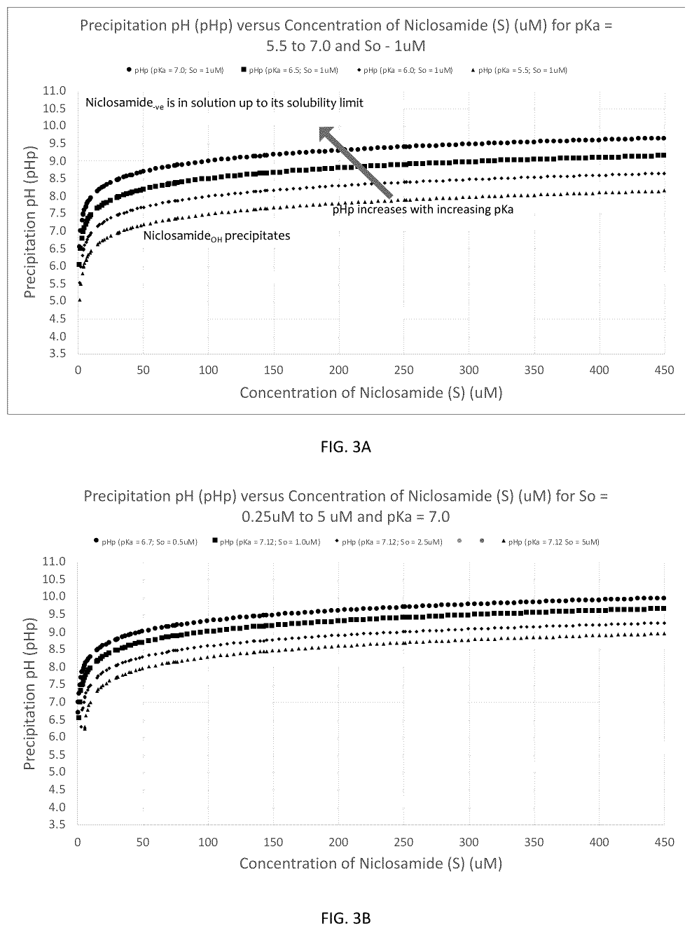

Changes in the amounts of niclosamide in solution brought about by alterations of solution pH can be predicted by the “pHp equation” as shown in FIGS. 3A and B. The Precipitation pH (pHp) is the pH below which an acid (or above which a base) will begin to precipitate (with all the necessary caveats associated with stochastic homogeneous precipitation when formed from supersaturation).

It is related to the pKa of the drug its intrinsic solubility and its concentrations in supernatant solution by the equation:

pHp=pKₐ+log(S−Sₒ)/Sₒ Eqn 4

where,

Sₒ=the molar intrinsic solubility of the undissociated acid (Nic_{OH}), and S=the molar concentration of the salt form initially added.

FIG. 3 shows an exemplary plot of this limit to niclosamide solubility (as pHp) versus Niclosamide concentration in supernatant solution for (A) a range of pKₐs from 5.5 to 7.0 and a constant nominal Sₒ of 1 μM; and (B) a series of solubilities of Nic_{OH} (Sₒ) of from 0.25 μM to 5 μM and a constant nominal pKa of 7.0.

What this graph shows is that the pHp provides a precipitation limit for the amount of niclosamide in supernatant solution as we might want for a throat or nasal spray. As indicated in FIG. 3A, in the region of the graph above the lines, Niclosamide is in solution, predominantly as Nic₋ᵥₑ the negatively charged salt (up to its solubility limit, measured later to be ˜640 μM at pH 9.5); below the lines, niclosamide precipitates.

Thus, with the two niclosamide species, there are lower and upper limits to solubility. In the pH range below ˜9.5, it is therefore the Nic_{OH} that precipitates and is the reason micronized niclosamide has such a limited amount in solution. Of course, the precipitation, when initiated from super saturated solution, will depend on the degree of super saturation and time allowed for the precipitation to happen if in this part of the plot. Such supersaturations and the nature of the precipitated material are therefore investigated below (Methods, 3.3.6 and 4. Results, 4.5).

As we will see in the results of later experiments, the intrinsic solubility Sₒ of AKSci niclosamide is measured to be ˜2.53 uM and with a value for the pKa of 7.12, we obtain a very good fit to the experimental data. The overall result though is that for a given niclosamide acid solubility, the pHp increases with increasing value for the pKa.

Similarly, as shown in FIG. 3B, a plot of pHp versus Niclosamide concentration for a range of Nic_{OH} solubilities (Sₒ) (0.25 μM to 5 μM) and a nominal constant pKa of 7.0, shows that the pHp increases with decreasing solubility of Nic_{OH}. As shown in FIG. 3B, as expected, Sₒ also affects the position of the curve, and does so to a similar extent as the range of possible pKas. Clearly, the accuracy of the solubility measurement at these very low solubilities is quite critical.

And so, with two fitting parameters we can fit any subsequent data with a value for the pKa, and one for the intrinsic solubility. Basically, the curve can be shifted to higher pHps by either decreasing the value of the intrinsic solubility of the material or increasing the value of the pKa. Thus, to what extent we can fit the data to theory depends on the accuracy of not only the measurement of concentrations as a function of pH, but also on the accuracy of the measured intrinsic solubility of the very low solubility Nic_{OH} species and, if available, on measuring or calculating the pKa itself.

### 2.4. Comparison Between HH Curves and pHp

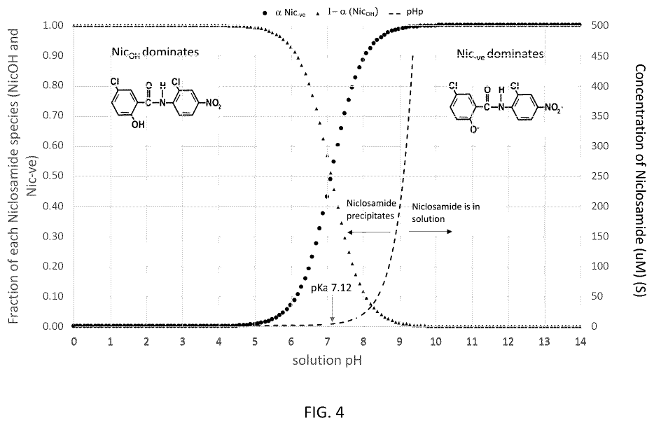

While this could be saved for the discussion when all results are presented, it seems more instructive to provide the comparison between HH curves and pHp here, in order for the reader to anticipate what is to come. Plotted in FIG. 4 using the values derived from experiment of pKa=7.12, Sₒ=2.53 uM are the fractions of each Niclosamide species (Nic_{OH} and Nic₋ᵥₑ) (left axis) and the pHp curve for the measured limiting solubility of niclosamide (uM), Sₒ versus the solution pH (right axis).

What this graph demonstrates is that the amount of niclosamide per se (as measured in the supernatants of dissolving material by UV/Vis) only starts to significantly increase beyond the pKa of 7.12. This implies that, at pH 7.12, although there is 50% of the highly soluble deprotonated salt present, the 50% of the low solubility acid dominates the precipitation. As measured by DLS PALS, any niclosamide precipitate is actually negatively charged at −15 mV and so some Nic₋ᵥₑ either must adsorb to the precipitate, or the pH at the plane of shear is not the bulk pH, but greater. Both curves are derived from the same basic equations:

HH in Eqn 1: pH=pKₐ+Log₁₀[Nic₋ᵥₑ]/[Nic_{OH}]

pHp in Eqn 4: pHp=pKₐ+Log₁₀(S−Sₒ)/Sₒ

Because the solubility limit for the (AKSci) Nic_{OH} is so low at 2.53 uM, therapeutically we are almost always dealing with a situation where there is some niclosamide that has precipitated or is present as micronized material. All this data was measured for the AK Sci material, and, as shown later, if a micronized formulation (such as that used by Brunaugh et al [91] obtained from Shenzhen Neconn Pharmtechs Ltd. Shenzhen, China) is actually the more stable hydrate it will can have even lower solubilities and reduced bioavailability as niclosamide in solution across the whole pH range.

In any event, below the solubility limit of Nic₋ᵥₑ (which is at or greater than 670 uM) and above the solubility limit of Nic_{OH}, (2.53 uM) the amount of NIC_{OH} in solution can only be at this limit of 2.53 uM, and the Nic₋ᵥₑ makes up the rest. Thus, as shown in FIG. 4, for a given pKa of niclosamide of 7.12, the position of this curve that delineates the solubility limits of niclosamide across the whole pH range, is determined by the intrinsic solubility value for the low solubility acid, in this case 2.53 uM. How these curves impact the amounts of niclosamide in solution for micronized material at ˜pH 5 and in solution up to pH 9.1 will be presented and discussed in Section 5.4.

The important conclusions from this analysis are that, if the niclosamide solutions are formed by dissolution of excess material, the pHp curve gives the amount of niclosamide in supernatant solution when at equilibrium with the undissolved material. Herein lies the issue and influence of crystal polymorphs and their own intrinsic solubilities, where the monohydrate is the most stable and therefore the lowest solubility, dictating the least amount of niclosamide in solution across the whole pH range. That is, it is the given value of So that determines the whole curve for a given pKa, which should be the same for all niclosamides that are actually dissolved.

### 2.5. Log P

Finally, the most important parameter with respect to mechanism of action of niclosamide on cellular membranes, is its Log D, which is the extent niclosamide partitions into octanol versus water as a function of pH. This parameter along with its size, (as molar volume) and its topological polar surface area determines its propensity to partition into lipid bilayer membranes (as Log B). Gobas et al [92] compared Log P and Log B for a series of 27 selected halogenated aromatic-hydrocarbons. Their data showed that, while the octanol-water partition coefficient (Log P) increases linearly with molar volume (Vs) the membrane-water partition coefficients (Log B), measured for DMPC bilayers, increased initially with Log P for the smaller molecules, but then followed a more parabolic relationship with respect to larger Vs. The maximum Log B occurred for solutes with molar volumes of ˜300 cm³/mol, after which, their ability to partition into bilayers went down.

For the case of such a small molecule as niclosamide, (Vs=202.5±3.0 cm³, ACD/iLabs) and a low topological polar surface area of 95.15 Å² (Drugbank), (where any value below 140 Å² is considered hydrophobic enough to partition into the membrane interior), it is well within the parameters for membrane partitioning. Thus, since we are focused on its pH dependence, it is now the Log D that is an indication of its ability to partition into lipid bilayer membranes and exert its proton shunt effect. That is, it is the extent to which the molecule is charged or not, since charges are not well partitioned into low dielectric (dc) media like the inside of lipid membranes where the dielectric constant is 2. It is here that niclosamide has its advantageous property, to delocalize the charge and become a lipophilic anion.

As given at ACD/iLabs, Log D niclosamide (for their calculated pKa of 6.89) is 4.55 at pH 0.01 and at pH 7.2 it is still 4.23. Above its pKa, the Log D is still 3.5. The reason its Log D does not decrease appreciably after it is deprotonated is, again, because the negative charge is delocalized within the molecule in internal hydrogen bonding, such that it becomes a lipophilic anion and so can act as the all-important intramembrane proton shunt [22].

### 2.6. Other Uncouplers of Oxidative Phosphorylation

As mentioned, Niclosamide came to the fore as an uncoupler of oxidative phosphorylation. In this activity, it is not alone. As reviewed by Terada in 1990, [39] there are several other molecular structures that have similar uncoupling effects. Phenols, benzimidazoles, N-phenyl-anthranilates, salicylanilides (niclosamide), phenylhydrazones, salicylic acids, acyldithiocarbazates, cumarines, and aromatic amines are known to induce uncoupling, by their protonophoric actions. For example, as given by Terada [39], other weakly acidic uncouplers (including the concentration of their uncoupling activity, acid groups, and pKas) include: dinitrophenol (50 uM, phenolic OH, 4.1); S-13, (30 nM, phenolic OH, 6.57); PCP, (1 uM), phenolic OH, 4.80); SF 6847 (10 nM, phenolic OH, 6.83); TTFB (100 nM, imidazole NH, 5.5 in 50% ethanol); flufenamic acid, (50 uM, COOH, 3.85); and FCCP, (100 nM, secondary amine, 6.2 in 10% ethanol). The most potent of these compounds are SF 6847 (3,5-Di-tert-butyl-4-hydroxy-benzylidene-Malononitrile) and S-13 a salicylanilide derivative, exhibiting uncoupling activity at concentrations in the 10 nM range. Thus, as niclosamide leads the way, there are other opportunities for drug and prodrug formulations as we have done with niclosamide, and hence similar preformulation characterizations.

Bottom line: This preformulation drug characterization has shown that Niclosamide has a relatively low solubility (1-2 uM) at neutral pH by virtue of the low solubility of the protonated Nic_{OH} species, which has a limiting solubility Sₒ(for the AK Sci material) measured below of 2.52 uM. However, upon deprotonation of its phenolic Cl-OH, it becomes a lipophilic anion. At pHs above its pKa (fitted here as 7.12, which agrees fairly well with the calculated value of 6.89 from ACD/iLabs and chemaxon), the negative charge is distributed within internal hydrogen bonding such that the molecule still has a Log D of 3.5. The reason this is important for drug delivery and intracellular action is that, while ostensibly charged, it can partition into lipid-cell membranes, including intracellular organelles, including the mitochondrial inner membrane. It can therefore act as a proton shunt and effectively dissipate pH gradients that the cell sets up across those membranes that usually drive a series of cellular functions including ATP generation, endosome acidification and assembly in the Golgi. It is this weak acid pKa of 7.12 coupled to the low intrinsic solubility of the Nic_{OH} (2.35 uM for the AKSci, and ˜1 uM for the monohydrate) though that enables the new and much higher concentrations of niclosamide to be made in simple buffer solutions. And there are other interesting compounds to also evaluate and test.

### EMBODIMENTS: INITIAL THINKING AND EXPERIMENTAL STUDIES

### 3.1. Embodiments

The overall scientific goal of this study was to explore the expected pH-dependence of Niclosamide implied by the literature and calculation databases for its pKa [22, 82, 84, 85]. As presented above, in the Preformulation drug characterization this data indicates that the solubility of niclosamide should increase with increasing pH as shown in FIGS. 2,3 and 4 and confirmed later in results, FIGS. 11A and 11B. It was also to then determine if, and to what extent, niclosamide could be actually extracted from commercially available and regulatory-approved tablets of niclosamide, namely Yomesan (Bayer, Leverkusen, Germany) as well as other generics (Luxiaoliunpian, Hanzhong Tianyuan Pharmaceuticals, China, data not shown). Finally, in support of the observations made in these experiments and data, given the unknown nature of the commercially available niclosamide material, it was to similarly explore different materials (as received from niclosamide suppliers, precipitates from supersaturated solution and recrystallized cosolvates), their dissolution and equilibrium solubilities and morphological changes that accompany these transitions as a function of solution pH.

There were, hence, a series of Embodiments.

### Embodiment 1. To Measure the Equilibrium Dissolution of Niclosamide Versus pH from a Commercial Supplier (AK Sci)

The first embodiment was to confirm that the amount of niclosamide (from a single supplier, AKSci CA) in buffer solution should increase with increasing pH by equilibrating small excesses of powdered niclosamide in stirred vials and measuring the supernatant concentration using a calibrated nanodrop and/or cuvette UV/Vis protocol.

### Embodiment 2. To Measure Rates of Dissolution of AK Sci Niclosamide, Sigma Niclosamide and Yomesan Powder as a Function of pH Using a New Methodology for Measuring the Supernatant Concentration In Situ

A new methodology was established for measuring the supernatant concentration of niclosamide in situ using UV/Vis nanodrop absorption of niclosamide solutions equilibrated with supplied powdered niclosamide and crushed and ground Yomesan tablets. Innovation here was to develop a new sampling technique that utilized a nanodrop UV/Vis spectrophotometer for detection of niclosamide at 333 nm and 377 nm using only 2 uL samples drawn directly from the stirred suspensions.

This new technique allowed the measurement of initial dissolution rates and full dissolution curves in situ as the drug was dissolving. Time periods were of 60 minutes and longer for a series of pH solutions using the AK Sci material and niclosamide from a second supplier, (Sigma) as well as the crushed Yomesan tablet. Rather than having to employ large volumes of suspension and a series of tubes, connectors, pumps, filters, and stirred cuvettes, 2 uL samples were taken directly from the 10 mLs of suspension in a stirred 20 mL vial. This allowed measurements to be made of the dissolution of the various niclosamide samples as a function of pH, in the pH range where soluble niclosamide can best be measured (8.5-9.5). This gave their initial rates of dissolution including more optimized mixing conditions for the supplied material (AK Sci and Sigma). As shown later, the reason this was important to compare is that, while the AK Sci material was relatively stable upon dissolution, the Sigma material converted to a lower solubility polymorphic form (probably the monohydrate) with overnight stirring.

### Embodiment 3. To Extract Niclosamide at pH 9.3 from Yomesan—Commercially Available and Regulatory-Approved Tablets

Niclosamide is available in 500 mg tablets that are taken orally for worms, and so the idea was to determine if and to what extent niclosamide could actually be pH-extracted (no organic solvent used) from such commercially available and regulatory-approved tablets of niclosamide including Bayer's Yomesan and a generic (Luxiaoliunpian, data not included). If so, the obtained niclosamide was very close to an already approved dosage composition and, if used as an oral spray, was essentially applied locally as a spray to the same epithelium but at lower doses (10 microgram) than are bioavailable (˜20 micrograms) when thoroughly chewed as (2 grams) Yomesan tablets.

### Embodiment 4. To Precipitate Niclosamide from Supersaturation and Obtain “Natural” Crystals at these pHs

When precipitated from supersaturated solution the precipitated microcrystals are expected to take on their “natural” morphology associated with nucleation and growth at that pH. Therefore, using the solvent injection technique it was possible to obtain such precipitated material and measure their equilibrium solubilities corresponding to precipitation at each pH. Optical microscopy also allowed images to be obtained of the particle morphologies at each pH.

### Embodiment 5. To Evaluate Dissolution of Niclosamide as a Water-Precipitate, and as Recrystallized Material from Acetone and Ethanol

The above studies led to a series of experiments that sought to provide additional information as to the nature of the commercially available niclosamide material that dissolved so readily (AK Sci and Sigma), where, in one case (Sigma) converted to a lower solubility form while in the other (AK Sci) was stable at high concentration for days in equilibrium with its original powder. Niclosamide was therefore precipitated into water to obtain the fully hydrated form and was recrystallized from ethanol and acetone and then dissolved to equilibrium in pH 9 buffer to determine their dissolution profiles, solubilities, and morphology, again by optical microscopy.

### Embodiment 6. To Measure the Equilibrium Dissolution of Sigma Niclosamide (3 Hrs and Overnight Stirring)

Finally, as was done for the AKSci material, embodiment 6 was to confirm that the amount of niclosamide from a different supplier, Sigma Mo, in buffer solution also increased with increasing pH. Here equilibration was again measured for small excesses of powdered niclosamide in stirred vials after 3 hrs and also after 24 hrs, equilibrating and measuring the supernatant concentration using a calibrated nanodrop and/or cuvette UV/Vis protocol. What was discovered here was that this form of niclosamide converted to the low solubility polymorph after overnight stirring because the starting material already contained this polymorph.

Bottom line: The picture that emerges from this series of Embodiments is one of niclosamide being able to take on different morphological forms as already established to some extent by van Tonder et al [5, 17, 88, 89], but now showing this for niclosamide from different suppliers, and over a range of pHs and concentrations. As discussed in more detail in the discussion section, any new formulations of niclosamide that are sought for testing in cells, animals, and especially in humans, and that are expected to deliver niclosamide when formulated for administration to, for example the nasal cavity as a nasal spray, need to take into account the intrinsic solubility of these more stable monohydrate forms. If they occur, or are allowed to occur, the nature of the polymorph will determine the amount of bioavailable soluble niclosamide that can permeate the protective mucin in a nasal spray or inhaled administration. In the niclosamide solution approach where the solutions are made by the ethanol-solvent exchange technique there are no undissolved materials and so the solubility-determining polymorphs are not present. As such, this new method of making the solutions means that the low solubility polymorphs cannot exert any deleterious equilibrating effects, i.e., the high niclosamide solution concentration is preserved.

### 3.2. Initial Thinking and Early Experimentation

For completion, this section briefly describes some initial thinking and early experimentation that generated a series of provisional patent applications [16] and guided the development of the simplest and most optimized formulation, the pH buffered solutions.

### 3.2.1. Niclosamide Readily Precipitates as a Fluffy Visible Precipitate

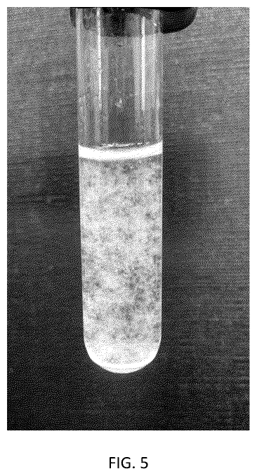

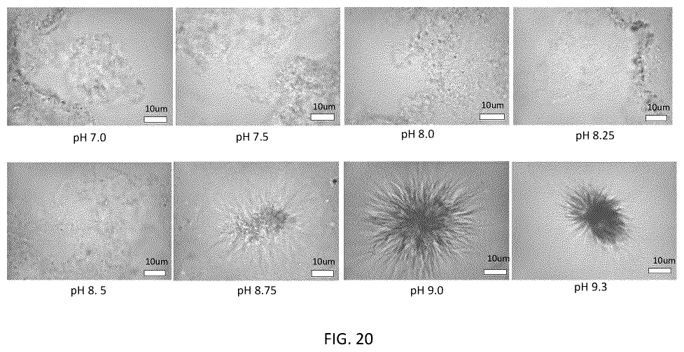

In the very first and simplest experiment, niclosamide was precipitated by solvent exchange (see 3.3 Methods 3.3.2) into excess aqueous (anti-solvent) buffer. As shown in FIG. 5, at physiologic pH, it forms large, and aggregated microparticles that present as a “fluffy”, gel-like, precipitate, that is visible by eye [3] (see microparticle images later in FIG. 20). The nasal mucosa is designed to protect the nasal epithelial cells and prevent the permeation of particles with a size greater than a few 100 nanometers [93]. Only small nanoparticles, micelles, and dissolved molecules of niclosamide in solution can get through.

In the initial thinking for formulation design for this nasal and buccal epithelial application, it was therefore feared that, while easy to make, particulate suspensions of niclosamide would simply be trapped at the top surface of the protective mucin layer and so would not be fully optimized for a nasal or throat spray. That is, microparticles that cannot penetrate the mucin barrier can only provide the small fraction of the low solubility niclosamide (1 uM-2 uM) in solution and so do not optimally deliver molecular-niclosamide to the underlying epithelial cells. However, microparticle formulations such as Nasonex and Flonase do exist for nasal applications, and so what is the situation here?

### 3.2.2. The Case of Nansonex and Flonase that could Inspire Microparticle Formulations

While the microparticle-micronized drug concept for drug delivery is clearly a well-established option, especially for nasal sprays, is it really optimized for niclosamide? It is here that considerations of crystal polymorphs, morphology and their pH dependent intrinsic solubility are key determinants in the design, optimization, and ultimate performance-in-service. As an example, in stark contrast to the 1-2 uM solubility of niclosamide and its 2-3 uM IC₁₀₀ virostatic efficacy [34], micronization does work for similarly low solubility drugs like mometasone furoate and fluticasone propionate i.e., as Nansonex [94-96] and Flonase [97-99] respectively.

Consider first the formulations themselves and the amount of micronized and soluble drug present compared to their efficacy. For mometasone furoate, according to the FDA IND approval [95], Nasonex Nasal Spray in aqueous medium, contains: 50 micrograms of the API mometasone furoate; Glycerin, microcrystalline cellulose and carboxymethylcellulose sodium, sodium citrate, citric acid (to control pH) benzalkonium chloride (preservative), and polysorbate 80. It has a pH between 4.3 and 4.9. Mechanistically, the molecule Mometasone furoate diffuses across cell membranes to activate pathways responsible for reducing inflammation. Each actuation of Nasonex delivers 50 mcg of mometasone furoate in 100 uL of formulation through the nasal adapter. Thus, the mometasone furoate concentration per spray (50×10⁻⁶ g/100×10⁻⁶ L)×1/521.43 g/mol)=958 μM. As micronized material this is in presumable equilibrium with its aqueous solubility of 20.7 uM (calculated from chemaxon).

By comparison, the relative trans-activation potency (EC₅₀) for mometasone furoate is 0.069±0.021 nmol/L, i.e., the amount required to induce GR-mediated transcription of a synthetic target gene regulated by a glucocorticoid response element is delivered at 14 million times more drug than its EC₅₀. Similarly for fluticasone propionate with an aqueous solubility of 22.8 uM, its EC₅₀ activity is 0.320±0.04 nmol/L and so Flonase delivers 3 million times more drug than needed for activity. As is clear, most of this drug is in the form of micronized mometasone furoate or fluticasone propionate, that, because of its few-micrometer size, cannot get through the protective mucin that covers the underlying epithelia. And so, the key mechanism here for drug transport is simply the diffusion of the soluble fraction, (21 uM or 23 uM) of drug that can permeate through the mucin. Thus, while Nasonex and Flonase might provide good examples and motivation for micronized drug formulations for nasal application, they only work because of the huge 30,000 to 6,000 times greater bioavailable aqueous concentration of the drugs compared to their nanomolar efficacy.

It's the amount of drug in solution at thousands of times the effective dose at the receptor that gets to the underlying cells to have its corticosteroid receptor action, while the 1 million times excess micronized drug just sits on the mucus that is being replaced every 21 minutes.

### 3.2.3. Unfortunately, Niclosamide's Solubility at Physiologic pH is in the Same Range as its Efficacy

The most stable form of niclosamide (its monohydrate) [17] is such a low solubility drug at physiologic pH, (1 uM-2 uM) and its anti-viral efficacy has been determined to also be in this same range of 1 uM [14] in Vero 6 cells and 2-3 times less in the more appropriate lung Calu-3 cells (˜2-3 μM) [34], Thus, at nasal pH (6-7) any aqueous solution of niclosamide that is in equilibrium with any particulate, for example, micronized niclosamide may not be sufficient for optimal efficacy. It really does not matter how much microparticles of niclosamide are sprayed intranasally, the bioavailability is essentially set by the solubility of the niclosamide polymorph in the formulation at the environmental pH. As discussed in more detail below (5.2.2) given that nasal mucosa has a refresh rate of ˜21 mins [100], even as a depot of material, microparticles would likely not dissolve sufficiently well or quickly enough in the limited amount of water present in the mucosa to provide optimal niclosamide to the underlying epithelia. Thus, while microparticle niclosamide may provide some efficacy because it does provide some but limited amounts of niclosamide in solution, it is clearly not an optimally bioavailable formulation.

### 3.2.4. Could Niclosamide be Encapsulated or Stabilized as Nanoparticles?

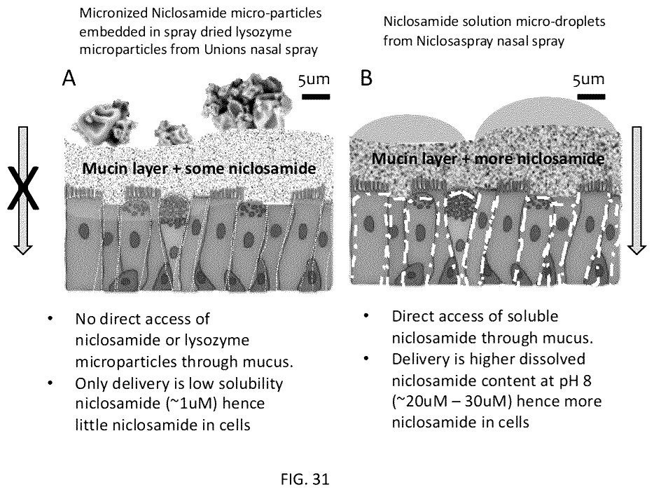

Since March 2020, our efforts were focused on ways to increase the amount of niclosamide in a sprayable suspension stabilized by a series of common surfactants, polymers, and preservatives routinely used in, for example, mouthwash, nasal sprays, and eye drops [16]. The goal was to make nanoparticles that could permeate the mucin, because, as shown schematically later in FIG. 31, with a particulate cut off of ˜0.5 μm [93] microparticles of ˜1-10 μm diameter would be unlikely to provide optimal delivery of niclosamide.

Attempts were therefore explored to stabilize the large visible particles, seen in FIG. 5, into something smaller that could stabilize niclosamide nanoparticles and so could in principle permeate the mucin. We have previously used a solvent exchange technique to introduce ethanolic niclosamide solutions into excess aqueous anti-solvent to form drug delivery particles for cancer [12, 13]. Using this technique for niclosamide and a range of surfactant, polymer and protein stabilizers, many well-stabilized micro- and even nano-particle suspensions were prepared. A series of provisional patent applications were obtained [16]. However, while of low aqueous solubility, niclosamide is still soluble enough for any precipitated nanoparticles to ripen and become larger microparticles, which, again, would merely stick to the top of the mucin layer and so be unsuitable (or at least not optimized) for nasal sprays. Also, as presented later in Results 5.1.4, and recognized and characterized by others [5, 17, 88, 89] “niclosamide is not niclosamide is not niclosamide”. That is, commercially available niclosamide that may be initially purchased and used in a formulation, but it is not necessarily the most stable and therefore least soluble form but can convert in aqueous media to its low solubility monohydrate polymorph [17] over time. What this means, formulation-wise, is that even if a microparticle formulation is made, if resuspended in aqueous buffer, it could readily convert to a low solubility monohydrate polymorph and so become less bioavailable in solution.

Examining individual surfactants in the commercial mouthwash and hydrating nasal sprays that are included as stabilizers and quaternary ammonium preservatives, gave micellized, microcrystals and also stabilized positively charged ionic-complexed nanoparticles (of ˜0.2 um-0.5 um in diameter). These latter particles could, in principle, adhere to the negatively charged mucus. However, they were found to be so stable that they would not sufficiently dissolve. As for micellar solutions (Tweens Poloxamers), while readily prepared, such relatively high surfactant concentrations could be toxic to the nasal epithelium.

Niclosamide can also bind to albumin in a 1:1 ratio [101, 102], and in other experiments it was discovered that if the concentration of albumin in solution was, for example, 15 uM, then niclosamide could be added to this albumin solution (by the ethanolic solvent exchange technique) and not precipitate. Dynamic Light Scattering (DLS) measurements showed that size of particles was 8 nm representing the diameter of macromolecules of albumin and so niclosamide simply bound to the albumin up to this 15 uM concentration. If the amount of added niclosamide exceeded this limit for albumin binding, the niclosamide did precipitate as ˜135 nm-150 nm diameter nanoparticles that were then stabilized by the albumin, as also measured by DLS [102]. In this way, it is also possible to create niclosamide solutions in solutions of albumin and additionally, suspensions of albumin-stabilized niclosamide nanoparticles that could all, in principle, permeate the mucin and so represent additional embodiments for nasal and throat sprays.

Again, this is not to say that a microparticle suspension stuck at the mucin surface could not provide some limited amount of niclosamide in equilibrium solution that could reach the underlying epithelial cells to some extent and show some efficacy, (as it has been encouragingly shown to do with micronized niclosamide [91]), it is just that this nasal delivery could be optimized by using a much simpler solution at higher but still tissue-tolerated pH. In contrast, 20 uM to 300 uM niclosamide solutions (with no microparticles) readily diffuse through and permeate the mucin to more-optimally deliver greater amounts of niclosamide to the underlying epithelial cells, as presented next.

### 3.2.5. A “Solution” to the Problem: How Much Niclosamide is Actually Sprayed Per Dose?

As described above (2. Preformulation drug characterization) and demonstrated in the results section (4.2), an evaluation of the physicochemical properties of niclosamide revealed a much simpler solution (literally); one that could be obtained by slightly increasing the pH (to pH 8) of the aqueous media that niclosamide was dissolved into. This provides niclosamide concentrations of 20 uM-30 uM that are 10× the efficacious virostatic levels of 2 uM-3 uM [14, 34] and that we have shown effectively reduce the ATP produced in host airway epithelial cells at levels that are also non-toxic to these cells [45]. Therefore, prophylactic nasal spray solutions of 20 uM are expected to be safely administrable and pH 8, itself, is also within the nasal pH range. Early treatment throat spray solutions of up to 300 uM niclosamide have been obtained at pH 9.2, which is on the same order as the pH of green tea or commercially sold alkaline water and so for oral administration this pH is also expected to be safe.

For the treatment of worms, 4×500 mg of Yomesan tablets are thoroughly chewed in the mouth or mixed to a paste in 30 mls of tap water and swallowed. In comparison to this, 2 grams of niclosamide that the buccal and throat epithelia is exposed to during Yomesan administration, for the throat spray, a single spray of 100 uL of 300 uM niclosamide is only 9.8 micrograms per spray, i.e., 204,000 times less niclosamide. In reality, it is not this dramatic a difference because, as is one of the main themes of this invention, we have to take into account what is actually bioavailable to the buccal epithelium as molecular dissolved niclosamide and not just particulate niclosamide tablet. So, what would this represent? A simple calculation provides the answer.

If the pH of tap water (and saliva) is ˜pH 6-7, then, as measured here in the Results section, the amount of niclosamide in solution at this pH is ˜2 uM. Therefore, the amount of niclosamide that could potentially dissolve out of the 2 grams of tablets in 30 mLs at 2 uM niclosamide, is 60 nanomoles or 19.6 micrograms (Mwt niclosamide is 327.1 g/mol). Compare this now to the 100 uL of 300 uM throat spray dose of 9.8 micrograms, which is similar, but still 50% less than the approved oral tablets. So why not just chew the Yomesan tablet? Because it is 204,000 times more niclosamide than you need, and, if swallowed, does have GI side effects that would make it somewhat intolerable on a regular basis and so not that patient compliant. In fact, this is the basis for the embodiment here that niclosamide can be readily extracted from existing approved commercial tablets, including Yomesan (Bayer) niclosamide tablets, to higher concentrations than in tap water or saliva if the pH is raised. Once prepared I this way and perhaps filtered to remove tablet excipients this solution could also be sprayed.

For the prophylactic nasal spray at 20 uM niclosamide, 100 uL of 20 uM niclosamide solution is only 0.65 ug per spray, i.e., 3 million times less in total mass and still ˜32× less in terms of bioavailable niclosamide. These simple calculations actually emphasize the main theme of this paper, that, intrinsic solubility, and the amount of niclosamide in solution is everything when it comes to epithelial bioavailability. It also highlights the need to identify the particular polymorph that is in equilibrium with any aqueous phase in a formulation since as shown here, niclosamide is supplied in different forms that have different intrinsic solubilities, some (AKSci) higher than others (Sigma)

Bottom Line: Thus, in this article, both the novel solution formulation and the reappropriation of existing commercially available tablets are described, and methods and data given to support their utility as nasal and throat sprays for the prophylactic and early treatment of SARS-COV-2 infections, its more contagious variants and other respiratory viruses. This niclosamide solution formulation has already been scaled up to multi-liter volumes as a 503b pharmacy batch that can be provided in 10 mL sealed and capped sterile vials; it just needs testing.

### 3.3. Materials

Niclosamide was from AK Sci (CA) (Lot No. 90402H, listed as, at least 98.9% pure by HPLC) and Sigma, St. Louis, MO; Water was deionized and filtered through 0.22 μm filters. TRIZMA buffer was from Supelco and comprised: Trizma Base, 99.8+%, reagent grade Tris(hydroxymethyl)aminomethane (HOCH₂)₃CNH₂, Mol Wt 121.14 g/mol, white crystalline powder; Trizma HCl, 99+%, reagent grade (Tris[hydroxymethyl] aminomethane hydrochloride), (HOCH₂)₃CNH₂·HCI white crystalline powder Mol Wt 157.60 g/mol. Ethanol 200% proof, and Acetone (VWR), and Dimethyl acetamide (DMA) (Sigma).

Trizma Buffer: Trizma HCI in solution produces a pH of approximately 4.7 but has little if any buffering capacity. Trizma Base in solution produces a pH of approximately 10.4 but also has little if any buffering capacity. Blending Trizma Base and Trizma HCI produces any desired pH between 7 and 9, with a reasonable buffering capacity.

pH buffers were made using the TRIZMA HCl and TRIZMA base buffer system (Supelco). An online calculator was used [103] where pHs between 7 and 9 and concentrations are specified. For example, 1 L of 0.1M buffer at pH 9 is obtained from the following recipe:
Prepare 800 mL of distilled water in a suitable container (1 L glass screw capped bottle).
Add 1.011 g of Trizma HCl to the solution (0.0064M).
Add 11.337 g of Trizma Base to the solution (0.0936M).
Make a series of 100 mL buffer solutions in 250 mL glass screw capped bottles nominally at pHs of 4.0, 7.0, 7.5, 8.0, 8.25, 8.5, 8.75, 9.0, 9.3 and 9.5 from this 1 L stock solution.

At the highest TRIZMA base-concentrations it was possible to exceed pH 9 and extend the range to pH 9.5. The pH solutions were made by adding to the stirred solution suitably small amounts of 2M HCl and fine-tuned with 0.1M using a pipettor to achieve the lower pHs or by adding small amounts of Trizma base to raise the pH. pHs were measured using a Mettler SevenEasy™ pH Meter S20, calibrated prior to any measurements using standard (VWR) buffers of pH 4, 7 and 10. Nominal pH 4 buffer was made using sodium citrate-citric acid buffer.

### 3.4. Methods

### 3.4.1. Solubility Versus pH: Calibration Standards

A calibration for niclosamide concentration (y) versus UV absorbance at 33 nm (x) was first obtained at 20° C. on a Thermo Scientific Nanodrop 1000 UV-Vis Spectrophotometer over the range 25 uM-300 uM niclosamide, providing a linear fit of y=0.0014x−0.0006. This nanodrop was used primarily for measuring the dissolution of the niclosamide samples versus time, taking only 2 uL from the stirred suspension to measure the supernatant concentration of dissolving niclosamide. As described here and this represents a new method and protocol for drug dissolution determination. However, for very low concentrations the extremely small pathlength in this nanodrop technique (0.5 mm) had relatively poor resolution, and it was difficult to distinguish, for example, 1 uM and 2 uM concentrations from blank controls. Equilibrium solubility and equilibrium amounts of niclosamide in buffered solutions were therefore carried out using the longer 10 mm path length provided by the cuvette in the UV5 nano (Mettler Toledo). This increased the resolution, accuracy, and reproducibility of the measurement of absorbance at 333 nm and hence the detection of niclosamide concentration. The calibration here gave a linear equation of y=0.0149x−0.0065, (R²=0.9998) and therefore provided for values in the 10 uM-200 uM range.

Both instruments were therefore used to measure and calculate the concentrations of niclosamide from their UV/VIS sample absorbance. In order to create the standards and provide accurate concentrations of niclosamide in solution, solutions were made by the solvent injection technique (see next 3.3.2). That is, weighing such small amounts to create 10 mLs or even 20 mLs of sample for the standards was difficult and even beyond the sensitivity of the mg balance (10 mLs of a 100 uμM Niclosamide solution requires weighing 0.327 mgs). However, for equilibration and dissolution experiments, such excess (mgs) of niclosamide were weighed and added to each buffer solution ensuring excess niclosamide that could dissolve and reach equilibrium.

It is here that it was discovered how one supplied niclosamide (AKSci) maintained its initial polymorphic state and provided relatively high concentrations of niclosamide in solution in equilibrium with excess powdered material, while other supplied niclosamide (Sigma) initially dissolved to a similar extent but then overnight converted to the low solubility presumably monohydrate polymorph. This was also the case for niclosamide precipitated from supersaturated solution that when precipitated also formed what appeared to be the low solubility polymorph as did dissolution of water, acetone, and ethanol co solvates.

### 3.4.2. Solvent Exchange Technique for Making Calibration Standards and Supersaturated Solutions (Used in Embodiment 4)

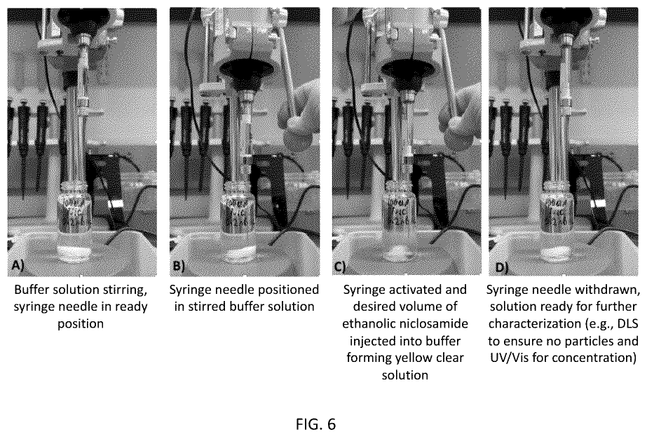

The solvent exchange technique, that we have normally used to make nanoparticles [12], can readily be used to make small volumes of niclosamide solutions as used in Embodiment 4, and indeed can be used to make the final solutions without including excess material by dissolution. The procedure is to simply inject a relatively concentrated ethanolic niclosamide solution into an excess of anti-solvent, —the stirred buffer. FIG. 6 shows the eVol syringe, 20 mL scintillation vial, and magnetic stirrer set up.

Basically, the technique involves diluting a concentrated ethanolic niclosamide solution and exchanging the good solvent (ethanol) for the antisolvent (pH buffer) but doing it in such a way that the final concentration does not exceed the pHp limit (as depicted in FIGS. 3 and 4 and shown later in Results, FIGS. 11A and B), and so there is no precipitation of niclosamide and therefore no polymorphs to equilibrate with. As shown in FIG. 6, an eVol syringe (Trajan Scientific and Medical. Trajan Scientific Australia Pty Ltd) is clamped securely in a commercially available drill press (Yeezugo, Guangzhou, China). This mounting allows for repeatable and accurate positioning of the syringe needle tip in the stirred solution, —a feature that is essential for injection of the solution into the most effectively stirred volume of the solution in the vial. While less of an issue when making solutions, a repeatable and consistent injection speed and mixing environment is critical for supersaturation precipitation since the vortex is not the most efficiently stirred part of the system and particle nucleation is very sensitive to the mixing environment [12].

The Protocol for Making the Standards is as Follows:

Withdraw 12 milliliters of the buffer solution into a 30 mL BD syringe. Insert and luer-lock a 0.22 um filter to the syringe and prime the filter by expelling 2 mLs of solution into a waste beaker. Add 9.9 mLs of the solution from the syringe through the filter into the 20 mL scintillation vial. Load the eVol syringe with 100 uL of a 30 mM niclosamide solution in ethanol that has also been filtered through a 0.22 um filter to remove any insoluble residual particulate material in the supplied niclosamide powder. Add an ethanol-cleaned, small magnetic stir bar to the vial and place the vial on a magnetic stirrer as shown in the photographic images in FIG. 6. Turn on the magnetic stirrer to create a vortex (FIG. 6 A). Bring down the syringe needle and position it over to the side of the vortex (FIG. 6B) i.e., for better mixing the syringe tip needs to be close to the ends of the stir bar. Inject the 100 uL of 30 mM ethanolic Niclosamide into the stirred solution at maximum injection rate (FIG. 6 C). Within 1 to 2 seconds of finishing the injection turn off the stir motor, raise the needle (FIG. 6 D) and remove the vial. This method makes 10 mLs of 300 uM Niclosamide including 1% residual ethanol.

Calibration standards (of 20 uM, 50 uM, 100 uM, 150 uM, 200 uM, 250 uM and 300 uM) for the nanodrop UV/Vis experiment were prepared by injecting 100 mL of ethanolic niclosamide solutions into 10 mLs of the high pH buffer giving a 1:99 dilution and a final 1% ethanol solution of niclosamide. To achieve each niclosamide concentration, a 30 mM stock solution was made by weighing 100.09 mg of AK Sci niclosamide powder (i.e., 98.13 mg of niclosamide, allowing for the 2% impurity that is filtered out later) and dissolving in ethanol up to 10 mLs total volume. Subsequent dilutions of 2 mM, 5 mM, 10 mM, 15 mM, 20 mM, and 25 mM ethanolic niclosamide were made in 2 mL final volumes, and 100 uL of each ethanolic niclosamide solution were injected into individual vials of 10 mLs of pH 9.3 buffer. This meant that there was a residual 1% ethanol in each standard. The Yalkowski model for solute solubility as a function of cosolvent-water mixtures (in this case ethanol) [104-106] shows that 1% ethanol would only increase the solubility of niclosamide by ˜4% compared to pure buffer. Actually, compared to simple weighing of powder directly into aqueous buffer, this technique provides a very convenient way to make more pure solutions, since the small amount of insoluble impurity that appears to be in the AK Sci product, and does not dissolve in ethanol, can be filtered out prior to making the solution using a 0.22 um filter.

This procedure can also be repeated using smaller injection volumes of the same 30 mM ethanolic niclosamide, e.g., 33 uL of 30 mM ethanolic niclosamide into gives a 50 uM solution, and then the % ethanol changes, in this case to 0.06% for a 1:600 dilution. A similar calibration was made for the UV5nano cuvette.

### 3.4.3. Equilibrium Dissolution of AK Sci Niclosamide Versus pH at 20° C. Used in Embodiment 1

A series of niclosamide solutions were prepared at each nominal pH from 4 to 9.5 (4, 7, 7.5, 8, 8.25, 8.5, 8.75, 9, 9.3, 9.5) by dissolving niclosamide at concentrations that were in excess of its expected solubility, i.e., 100 uM (0.33 mg/10 mLs) for pH 4, 7, 7.5 and 8; 300 uM (1 mg/10 mLs) for pH 8.25, 8.5 and 8.75) and 1 mM (3.3 mg/10 mLs) for pH 9, 9.3 and 9.5. As can be appreciated, very small amounts were required, (˜0.33 mg to 3.3 mgs). Niclosamide, as obtained directly from the supplier (AK Sci) was weighed into a weight-zeroed 1.5 mL Eppendorf tube and added to 10 mLs of each buffer solution in 20 mL screw top scintillation vials at 20° C.

The solutions and undissolved particles in the vials where then shaken by hand to wet and immerse the hydrophobic niclosamide powder and stirred by magnetic stirrers overnight to reach the solution equilibrium at 20° C. Measurements were made a few hours after making and stirring, and after 1-8 days to ensure equilibrium. Since some suspensions were visibly cloudy due to the excess undissolved material, in order to avoid sampling particles that could interfere with the UV/Vis measurement, 0.5 mLs of the supernatants were taken, spun down by centrifugation (10 mins at 15,000 G) to remove any suspended particles, and analyzed by UV/Vis nanodrop spectrophotometer (ThermoFisher 1000) and UV5nano (Mettler Toledo) cuvette systems. Full spectra were recorded and the absorbance at the 333 nm peak was compared to the calibration in order to determine the niclosamide concentrations of the supernatant. Their final pH was remeasured on a Mettler Toledo pH meter.

For the UV/Vis measurements, each buffer was used as the blank, and at least 5 individual samples were taken for each buffer and averaged to establish the “blank baseline” which was usually between −0.003 to 0003 absorption values. These values were subtracted from the niclosamide measurements. This subtraction was particularly important for the very low niclosamide concentrations at pHs 3.66 and 7, and 7.5, especially for the lowest solubility monohydrate polymorph, where the signal was very close, but not in distinguishable, from the usual noise of the instrument (˜0.003 absorbance units).

### 3.4.4. Rates of Dissolution for AK Sci Niclosamide, Sigma Niclosamide and Yomesan Powder Used in Embodiment 2

The dissolution of niclosamide and Yomesan powder at 20° C. was measured in a timed dissolution study using the nanodrop spectrophotometer. Appropriate amounts of AK Sci niclosamide powder, as in the above equilibration studies, were weighed into dry 1.5 mL Eppendorf tubes and capped. Since, as shown in results, final concentrations of niclosamide for pH 7 to 8.25 were still relatively low, only pH 8.5, 8.75, 9, 9.3 and 9.5 were tested. 10 mLs of each buffer were aliquoted from a 10 mL BD syringe fitted with a 0.22 um filter into 20 mL scintillation vials, and a magnetic stirbar added to the vial. At time (t)=0, the contents of one of the Eppendorf's was rapidly emptied into the pH 8.5 solution vial. The vial and solution were quickly shaken in order to wet and immerse the hydrophobic powder into suspension, and the vial was placed on the magnetic stirrer. 2 uL samples for UV/Vis measurement on the nanodrop spectrophotometer were taken by a 2-20 uL pipettor at regular time intervals, e.g., every 30 s up to 7 minutes, every minute from 8-16 mins, every 2 mins from 18-30 mins and then every 5 mins from 30-60 mins. Thereafter, samples were taken at 90, 120, and 180 mins, as well as the next day.

Thus, by utilizing 2 uL sampling and making measurements on the nanodrop spectrophotometer a new technique for measuring drug dissolution that does not require large volumes, pumps, filters and stirred UV/Vis cuvettes was developed. It seemed that by extracting only 2 uL from the stirred supernatant it was very rare for the 2 uL volume to include a powder particle. In any event, if this happened, the nanodrop software identified it as perhaps a bubble or error due to changed path length and the measurement could be excluded and repeated.

### 3.4.5. Niclosamide Extraction from Yomesan Tablets at pH 9.3 Used in Embodiment 3

Direct dissolution, and hence extraction, of niclosamide from commercial tablets into buffer at 20° C. used the already approved materials from Bayer's Yomesan. In this experiment, it was determined if and to what extent the niclosamide embedded in the Yomesan tablets could be extracted into pH 9.3 buffer by simply grinding up the tablet into a relatively fine powder (using a mortar and pestle) and adding excess powder to the buffer solution. A 500 mg niclosamide tablet weighed 668.16 mg, and therefore 161.8 mg of the tablet was binder, filler, and other excipients. The tablet material was therefore 0.748 mg niclosamide/gm of tablet. The equivalent amount of powdered to give a 1 mM suspension niclosamide was weighed (3.3 mg niclosamide/10 mLs; actually 4.37 mg allowing for tablet excipients) and equilibrated in a stirred buffer solution as described above. According to Bayer's literature [107], the excipients are: maize starch, talcum, sodium lauryl sulphate, povidone, vanillin, magnesium stearate, saccharin sodium, some of which are soluble in water, and some are insoluble.

As with the pure niclosamide, dissolution and equilibration were carried out in the magnetic-stirbar-stirred vial. Sampling for UV/VIS absorption measurement was done at ˜3 hrs, and the suspension was left stirring overnight. As described in results, this produced a more-cloudy suspension probably due to excipients. To recover the dissolved niclosamide in a clean solution, the 3 hr suspension was filtered through a 0.22 um filter and this supernatant concentration again measured by UV/Vis.

### 3.4.6 Supersaturated Solution Precipitation of Niclosamide: Amounts of Niclosamide in Solution at Each pH and Corresponding “Natural” Morphologies (Used in Embodiment 4)

An interesting experiment was carried out that determined the solubilities corresponding to what are expected to be the “natural” morphologies of niclosamide when precipitated at each pH at 20° C. Using a series of stock solutions in ethanol, final supersaturated concentrations of niclosamide were created in each pH buffer in slightly excess amounts, i.e., just enough to generate precipitated material given that time was required for the stochastic nucleation and precipitation to occur. This excess, kinetically soluble niclosamide was mixed into the buffers using the solvent injection technique. 33 uL of 30 mM into 10 mis of buffer gave supersaturated solutions of 100 uM for pH 3.66, 7, 7.5, 8, 8.25; 100 uL of 30 mM gave a 500 uM supersaturated solution for pH 8.5 and 8.75; and 333 uL of 30 mM gave a 1 mM supersaturated solution for pH 9.0, 9.3 and 9.5. The solutions were again stirred until precipitation was observed, often overnight and sometimes longer. In the case where no precipitate was seen, for example, in the pH 9.5 solution after 1-2 days stirring, additional volumes of 30 mM ethanolic niclosamide were added and the solution continued stirring until a precipitate was obtained. For this pH 9.5 sample, solution concentrations had to be increased to 3 mM supersaturation and even then, it was stable for at least a few hrs at 3 mM niclosamide. The precipitated suspensions were examined by optical microscopy using bright field optics; Kohler illumination and a 40× objective to obtain the micrograph images of the precipitates. 500 mL of supernatants were again centrifuged in 1.5 ml Eppendorf tubes for 10 mins at 15,000 G to obtain clear supernatant solutions that were in equilibrium with the various precipitated morphologies. Again, UV/Vis spectra and 333 nm intensities were obtained by the nanodrop spectrophotometer.

### 3.4.7. Dissolution of Niclosamide Solvates at 20° C. (Used in Embodiment 5)

To start to evaluate the various expected polymorphs and to try and obtain a morphology that was perhaps like the purchased materials (of unknown processing), three samples were made i.e., the “water-hydrate” precipitated from ethanol into excess water and two that were recrystallized from acetone and from ethanol.

Water precipitate: Niclosamide was precipitated from supersaturated solution by solvent exchange into excess deionized water by injecting 400 uL of ethanolic 25 mM niclosamide solution into 10 mLs of stirred pH 9.3 buffer, i.e., much as might be done after synthesizing the niclosamide dissolving in an ethanol solvent and recovering by precipitation into excess water. The niclosamide precipitated immediately as its usual “fluffy” white precipitate. It was filtered on a sintered glass filter, washed three times with deionized water, and dried. The material was ground by mortar and pestle ready for the dissolution test and viewed microscopically to evaluate its crystal morphology.

Niclosamide Recrystallized from Acetone and Ethanol. Niclosamide was recrystallized from acetone and ethanol by dissolving excess niclosamide into the solvents at 25 mM, gently warming in a fume hood to dissolve excess niclosamide, boiling off 50% of the solvent, that was then allowed to cool under stirring. The recrystallized niclosamide solvates were filtered and dried. Each material was also viewed microscopically to evaluate their crystal morphologies.

Dissolution of the solvates. As with the AK Sci, Sigma and Yomesan materials, ˜3.3 mg of niclosamide from each of the hydrate and cosolvates was weighed and added to stirred pH 9.3 buffer to give a nominal 1 mM niclosamide in suspension ready to dissolve. 2 uL aliquots were taken directly from the stirred solution in the 20 mL scintillation vial at appropriate time intervals and absorbance at 333 nm was measured by nanodrop spectrophotometer.

### 3.4.8 Dissolution and Conversion of Sigma Niclosamide to the Low Solubility Form (Used in Embodiment 6)

A final experiment evaluated the dissolution of niclosamide from a second supplier, Sigma, over the pH range 7 to 9.5 at 20° C. This powder was much lighter in color, a creamy yellow, as opposed to the more brown-yellow of the AK Sci product. As before (3.3.3 Equilibrium Dissolution of AK Sci Niclosamide versus pH) excess niclosamide was weighed to give an equivalent of 100 uM (0.33 mg) for pHs 7, 7.5 and 8.0; 200 uM (0.65 mg) for pHs 8.25 and 8.5, 500 uM (1.65 mg) for pHs 8.5 and 9.0 and 1 mM (3.3 mg) for pHs 9.3 and 9.5. When ready, each of these samples of powdered Sigma niclosamide was added to individual 10 mLs of buffer solution in 20 mL stirred vials, and the suspensions were stirred by magnetic stirbar for three hours. At this point 0.5 mls of each were centrifuged at 15,000 G for 10 minutes and the supernatant niclosamide concentration was measured by UV/Vis nanodrop spectrophotometer taking 2 uL samples. At least 5 samples were taken to give the average niclosamide absorbance. Each pH buffer was used to blank the measurements and the blank values (typically 0.002 or 0.003) were subtracted from the niclosamide absorbance values.

### 4. Results

### 4.1. Calibration Standards

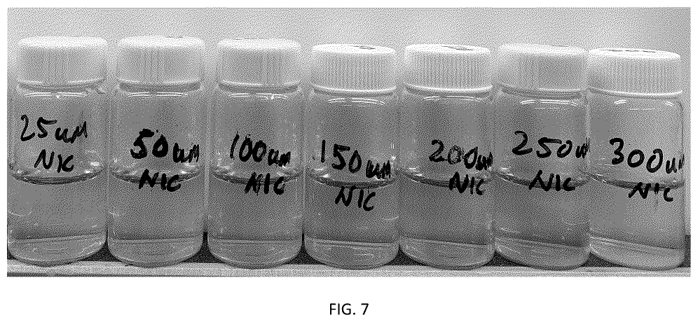

Calibration standards were made up in 10 mL volumes in 20 mL scintillation vials at nominally 20 uM, 50 uM, 100 uM, 150 uM, 200 uM 250 uM and 300 uM concentrations. FIG. 7 is a photographic image of the series of calibration standards made by solvent exchange technique in pH 9.3 Trizma buffer. When viewed as a color image, it shows, quite visually, how the concentration of niclosamide creates a greater yellowness with increasing pH of the solution.

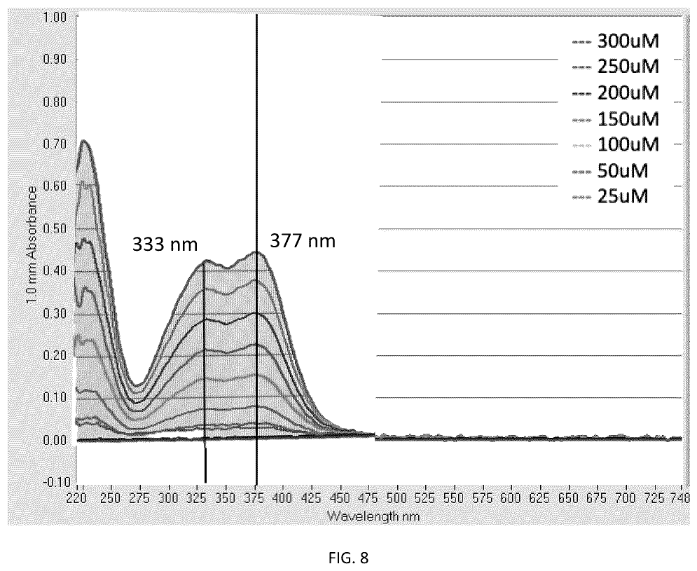

UV/Vis Absorption spectra are shown in FIG. 8, from which the average absorption values were obtained at 333 nm and 377 nm at 20° C.

As shown in FIG. 8, the niclosamide UV/Vis spectra from 25 uM to 300 uM all show the characteristic double peak profile at this pH of 9.3, with λₘₐₓ maxima at 333 nm and 377 nm. Measurement of niclosamide concentration was therefore taken at 333 nm, the most constant peak associated with niclosamide absorption.

It is unusual for niclosamide spectra to be obtained in aqueous media because of its relatively low solubility at normal working neutral pH of 7. Here we introduce for the first-time spectra for niclosamide at higher pH ranges.

Because niclosamide is usually thought to be relatively insoluble in aqueous media, calibration spectra are usually obtained in methanol, methanolic 0.1 N HCl and methanolic 0.1 N NaOH, as shown by Al-Hadya and Badraddin (Al-Hadiya 2005). The dominant peaks in methanol are at 333 nm and 377 nm. While niclosamide in methanolic 0.1 M NaOH continues to show the double peaks, in methanolic 0.1 M HCl where the salt is lost and the prevalent species is the protonated acid, there is only a dominant 333 nm peak. And so, the constant peak at 333 nm was chosen to report on niclosamide concentration in aqueous solution.

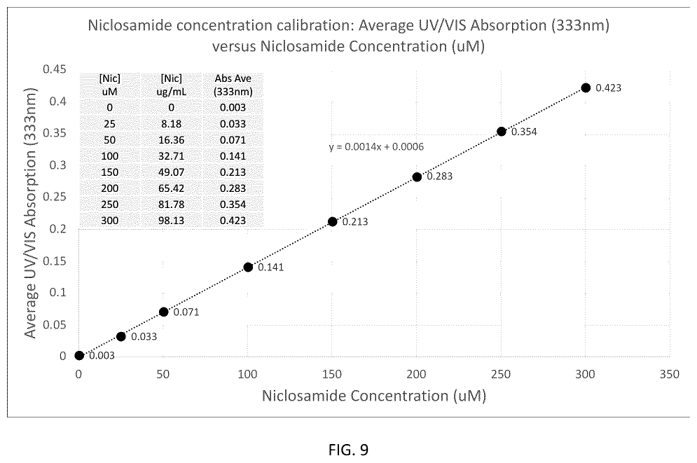

FIG. 9 shows the UV/Vis Absorption calibration at 333 nm (averaged for at least 5 measurements) and plotted versus Niclosamide Solution Concentration (uM) measured by Nanodrop at 20° C. The line is given by y=0.0014x+0.0006. Each absorbance values (Abs_{Nic}) were therefore converted to niclosamide concentrations [Nic] (uM) using this calibration equation, i.e., [Nic]=(Abs_{Nic}−0.0006)/0.0014. Equivalent values for ug/mL are given in the table inset. A second calibration on the UV5 nano using the cuvette (longer path length) was y=0.0149x−0.0065.

What follows now are the main Embodiments in more experimental and theoretical detail with analyses, interpretation and their relationship and impact to the nasal and throat sprays

### 4.2. Embodiment 1: Equilibrium Dissolution of Niclosamide Versus pH from a Commercial Supplier (AK Sci)

The vials containing the equilibrated niclosamide solutions measured by UV/Vis at 333 nm peak (by Thermo Scientific 1000 Nanodrop Spectrophotometer and Mettler Toledo UV5nano) as equilibrated stirred samples are shown in FIG. 10. It is again clear (when viewed in color) that the characteristic yellow solution coloration gradually increases with increasing pH. A 300 uM sample “standard” made by ethanol injection is shown for comparison (far right). In the final formulation, preparing the niclosamide-in-buffer solutions by ethanol injection of an ethanolic niclosamide solution into the buffer is preferred, since this ethanolic solution can be pre-filtered and is sterile and is more accurate for smaller volumes like 10-20 mLs.

For convenience, shown in Table 1 are the measured pH and average supernatant Niclosamide concentrations [Nic] (uM) corresponding to each of the vials in FIG. 10.

TABLE 1
Supernatant concentrations of AK Sci Niclosamide (uM)
measured by UV/VIS (UV5Nano cuvette, five measurements)
for each measured supernatant solution pH, after
stirring to equilibrium (48 hrs) at 20° C.

| | Average [Nic] (uM) | |
|---|---|---|
| Measured pH | UV/Vis, 333 nm) | Std Dev [Nic] (uM) |
| 3.64 | 2.53 | ±0.09 |
| 7.05 | 8.68 | ±0.13 |
| 7.64 | 16.75 | ±0.06 |
| 8.06 | 25.83 | ±0.12 |
| 8.26 | 47.26 | ±0.18 |
| 8.53 | 83.31 | ±0.17 |
| 8.75 | 137.29 | ±0.66 |
| 9.04 | 215.66 | ±1.46 |
| 9.22 | 344.32 | ±3.84 |
| 9.32 | 382.68 | ±3.65 |
| 9.4 | 466.14 | ±2.93 |
| 9.63 | 703.26 | ±13.06 |

Averages and Standard Deviations were taken from five UV/Vis measurements at each pH and the blank subtracted, (additional re-checks to the original series were also taken (not shown) in the steep part of the curve at pHs 9.1, 9.3 and 9.5). After addition and equilibration of niclosamide in solution, the pH values were fairly stable compared to the nominal pH. The concentrations of these same supernatant solutions are plotted in FIG. 11A for each sample. With increasing pH from 3.66 to 9.63 there is a concomitant increase in the supernatant concentration of niclosamide and this data is well fitted in form and position by the pHp curve Eqn 4, using the measured intrinsic solubility of niclosamide at pH 3.66 (Sₒ1) of 2.53 uM and a value for the pKa of 7.12.

In order to evaluate the data and compare to theory in what is, perhaps, a more easily evaluated form, the same data as in FIG. 11A is replotted in FIG. 11B with the axes switched. As can be seen, the supernatant niclosamide concentration ([Nic] uM) increased slowly over the lower pH range from 3.66 to just above 8, but then showed the expected more rapid rise in concentration from 8.5 to 9.63, where some of the highest values measured (and re-checked) for equilibrated samples were over 700 uM. This clearly demonstrates the potential for creating simple solutions of niclosamide.

Also included in FIGS. 11A and B is the pHp theory for a fitted pKₐ of 7.12 and measured limiting intrinsic Nic_{OH} solubility of 2.53 uM. The added lines in FIG. 11B show that, for prophylactic use, a 20 uM prophylactic solution can be made at pH 7.96, which is within the normal pH of the nasaopharynx [108]. For the early treatment throat spray, as preclinical animal studies and then human studies proceed to dose escalation, a 200 uM solution is readily achieved at pH 9.01, and the solution concentration of niclosamide can actually be raised to 300 uM at only pH 9.19. In the oral cavity a higher pH is expected to be tolerated and this is where a higher niclosamide concentration is perhaps required for already infected and shed or localized epithelial cells (see later discussion 5.2.3 The effects of niclosamide). Also, at pH 9.63, where the percentage of the deprotonated salt, Nic₋ᵥₑ, according to the HH plot in FIG. 2, is 99.2%, the amount of niclosamide in solution is now approaching the solubility limit for the Nic₋ᵥₑ which is at least 703.6 uM.

These data in FIGS. 11A and B are also compared to the theory for precipitation pH (Eqn 4). In order to compare the data to the theory, both the pKa and the limiting solubility of the acid component, Nic_{OH}, are needed. The value of 2.53 uM for AKSci niclosamide at pH 3.66 although not the low solubility form, is just slightly higher than the apparently lower limiting solubility of the Niclosamide Monohydrates H_{A} (2.9 μM; 0.95 ug/mL) and H_{B} (1.86 μM; 0.61 M) from vanTonder [17] measured in deionized water (pH not given but expected to be ˜6.5). Also, the data is fit to a pKₐ of 7.12 which is within the range of pKa data in the literature, (5.6 to 7.2, where the average of all these values is 6.52.)

Referring back to FIG. 4, where the Henderson Hasselbalch curves were compared to the pHp curve, the data are actually consistent with the prediction that at the pKa, where 50% of niclosamide in solution is the low solubility acid and this is in equilibrium with an equal amount of the salt, the total amount in solution should be controlled by acid solubility, i.e., the amount of the acid in solution is measured to be 2.53 uM, and so the total solubility at pH 7.12 should be 2×2.54 uM=5.08 uM, and the measured value at 7.05 is actually 8.68 uM.

As an aside, at pH of 9.7, 10.0 and 10.45 obtained by using a carbonate-bicarbonate buffer system, initial solutions were characteristically yellow and of high niclosamide concentration. However, over a short, 1 hr of stirring, the added niclosamide powder converted to red crystalline micro-crystals with a needle-like morphology reminiscent of the monohydrate, (but red). The presence of these crystals reduced the supernatant concentration, and so pointed to the appearance of a more stable and less soluble polymorph of niclosamide at these higher pHs, that appears to have not been recognized or reported previously. Further exploration of this polymorph is beyond the scope of the current work but could form the basis of future experimentation.

### 4.3. Embodiment 2: Rates of Dissolution for AK Sci Niclosamide and Sigma Niclosamide Powder

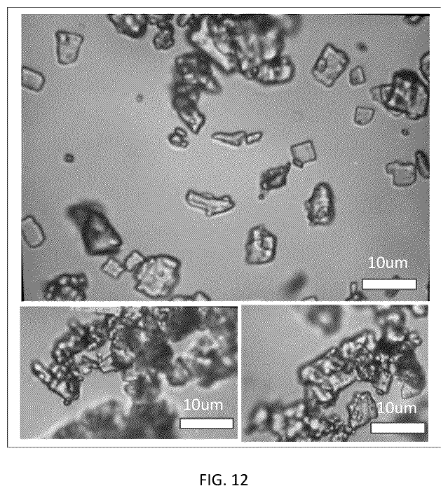

Having measured the equilibrium values of the amounts of niclosamide that can dissolve into supernatant solution over a range of pHs, the next series of experiments were to quantify the rates of dissolution of the AK Sci powder that was ground and suspended in water. First though, it is instructive to examine the initial morphology of the, as-supplied, AK Sci niclosamide powder. In FIG. 12 are photographic microscope images of AK Sci niclosamide powder after grinding with a mortar and pestle, resuspending in water to disperse, and bath sonicating to help break up aggregates.

Characteristic sizes, as length and width of individual particles range from 1-10 μm with an average size (from this micrograph) of 4.3 um+/−2.2 um. Interestingly, any rod-like needle-shaped and spiky crystals that are characteristic of the low solubility monohydrate, are noticeably and importantly absent as shown in the literature by van Tonder et al [5] as well as in subsequent images in this study (see FIGS. 20, 23, 26 and 27). This is consistent with the AK Sci product being a polymorph (perhaps a solvate) with a higher solubility than the most stable monohydrate, although its origins are unknown. While efforts were made to ask for details from AK Sci (and Sigma), suppliers were reluctant to divulge any information about product manufacture and post synthesis processing such as precipitation or recrystallization conditions, solvents, and potential solvates. Thus, the nature of the powdered material obtained from AK Sci is, as yet, unknown.

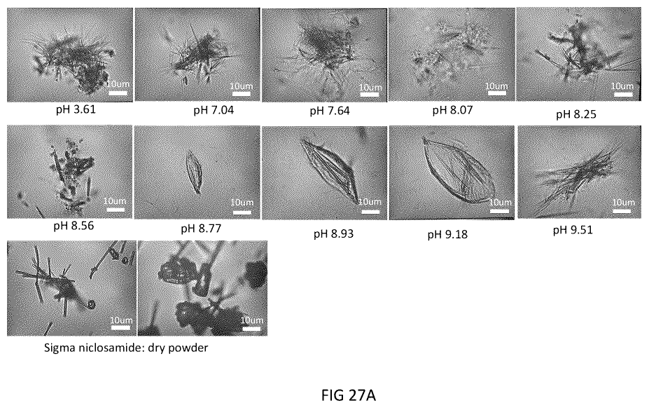

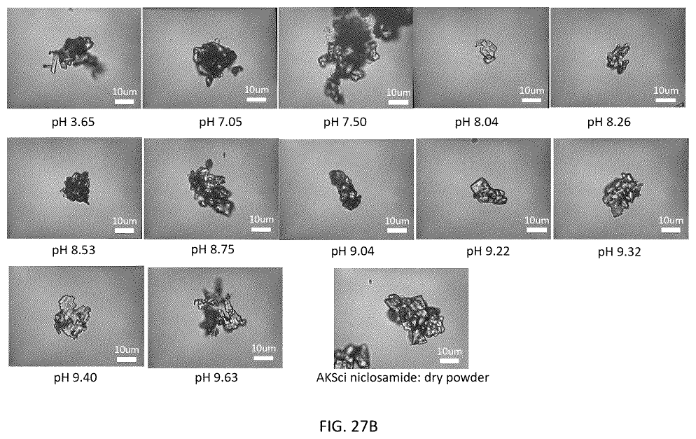

Upon dissolution of the AK Sci material, i.e., when a portion of the added (1 mM) niclosamide powder has dissolved and it was maintained under stirred conditions for 24 hrs and longer, its morphology did not change radically from the original powder. Thus, the AK Sci niclosamide did not readily convert to the less soluble hydrates when incubated for days in the presence of its original powder. Additional images across the whole pH range are given in FIG. 27 comparing the morphology of undissolved and equilibrated crystals of niclosamide from AKSci with those from Sigma. This presence of a higher solubility polymorph (as in AKSci) versus a lower solubility (monohydrate) polymorph as in Sigma, is clearly important to the bioavailability of soluble niclosamide in drug formulations that utilize microcrystalline or even nanoparticles of niclosamide in suspensions.

While a more quantitative analysis would measure the surface area per gram of drug powder, all samples were ground to a fine powder with the pestle and mortar (as shown in FIG. 12). So, for the same material (AK Sci niclosamide) the only variables are total mass and mixing. Samples were weighed to within 10% of 3.27 mg, which in 10 mLs gives a total equivalent 1 mM niclosamide, which is in excess of all expected saturated supernatant concentrations.

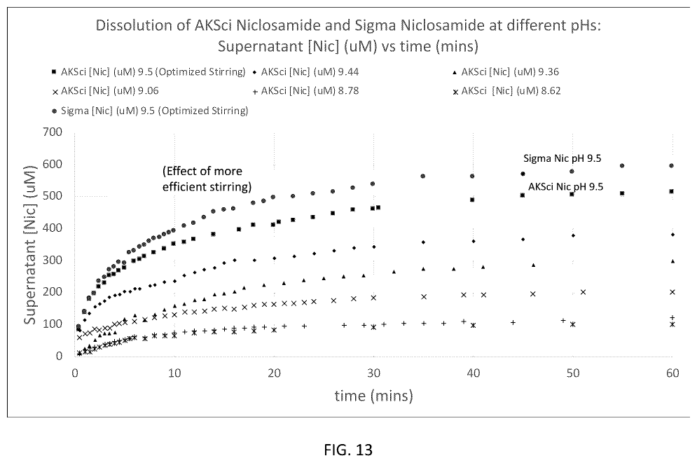

Therefore, since the mass added and mixing were similar for each pH sample tested, FIG. 13 shows the dissolution curves over the first hour, that give a fairly quantitative measure of intrinsic dissolution of niclosamide at each pH. As shown earlier in FIG. 11, below pH 8.5, the equilibrium saturation concentrations are still quite low, and so we focus here on the higher pH range 8.62-9.5 for a nominal 1 mM of the AK Sci material. The fitted logarithmic rate for each dissolution curve, as shown on the graph, was found to increase with increasing pH from a pre-ln factor of 22 at pH 8.62; 24 at pH 8.72; 38 at pH 9.06; and 71 at pH 9.36.

During the experiments for pH 8.62-9.5, the magnetic stirbar was relatively small compared to the diameter of the scintillation vial, (about half) and the rotation speed was medium. To (empirically) explore the effect of stirring speed, the size of the magnetic stirbar was standardized to about 75% of the diameter of the vial keeping the stirring rate at medium. Under these conditions, the dissolution rate of niclosamide in pH 9.44 buffer was significantly increased by about a factor of 33% i.e., 71 ln(x) versus 94 ln(x). For all subsequent dissolution studies, this would be the “standardized” set up. Using this standardized set up the dissolution of the Sigma niclosamide was made under these same, more optimized, stirring conditions. As also shown in FIG. 13, its rate of dissolution (116 ln(x)) was comparable to that of the AK Sci powder. More efficient stirring is shown by the filed symbols; AK Sci niclosamide reached 564 uM at 3 hrs and Sigma niclosamide reached 680 uM at 3 hrs.

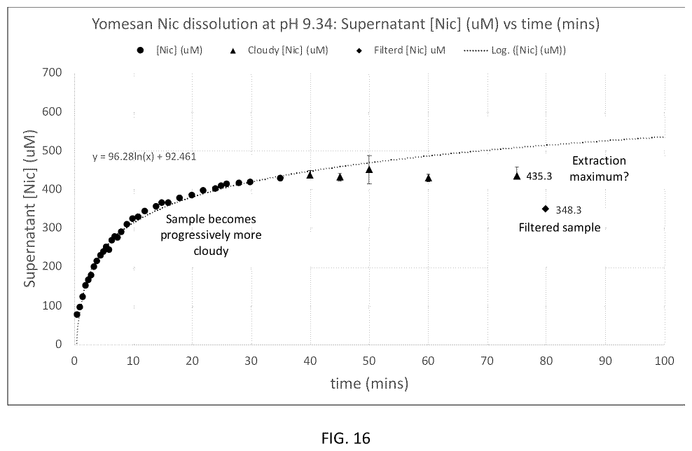

Also shown is dissolution in pH 9.33 of Yomesan tablet powder that was crushed by mortar and pestle under the optimized stirring conditions (see also FIG. 16). Interestingly, if left stirring overnight the supernatant niclosamide concentration decreased consistent with the formation of a more stable and lower solubility polymorph.

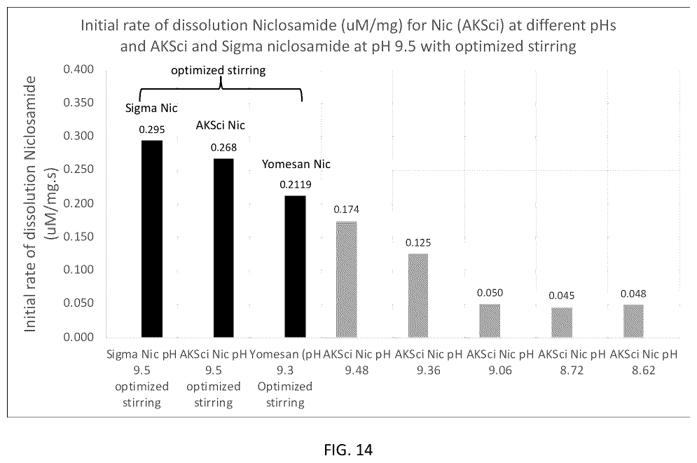

Consider now the initial dissolution rates over the first 3 min as intrinsic measures, i.e., in concentration units as micromolar per milligram·second (μM/mg·s); (micromolar implies micromoles/liter). FIG. 14 shows the initial (linear) dissolution rates from the data in FIG. 13, plotted as a bar graph. Consistent with the overall dissolution profiles, initial dissolution rates increased with increasing pH of the supernatant (grey bars). Also shown is the effect of the more optimized stirring for AK Sci and Sigma at pH 9.5 as expected from dissolution models (black bars). This more optimized stirring increased the initial rate of particle dissolution (empirically) by a factor of about 50% but did not change the final equilibrium saturation (solubilities). Also shown here for comparison in FIG. 14 is the rate of initial extraction of niclosamide from Yomesan, (again, see later, FIG. 16, for the full dissolution curve and filtration of the sample). This dissolution data, as suggested earlier, now demonstrates that niclosamide is available for dissolution even from ground-up commercially available niclosamide tablets.

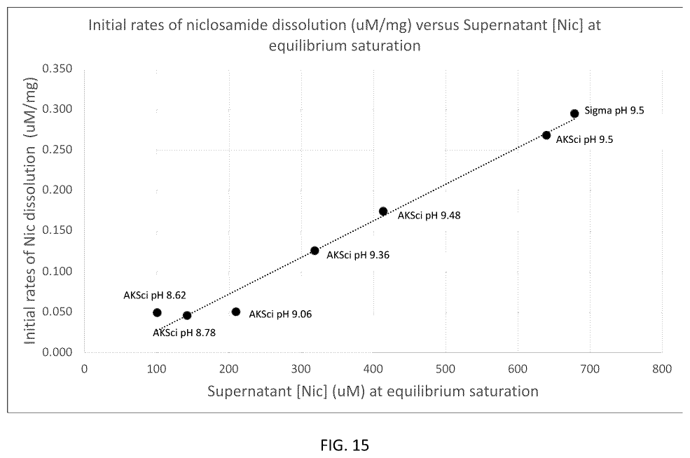

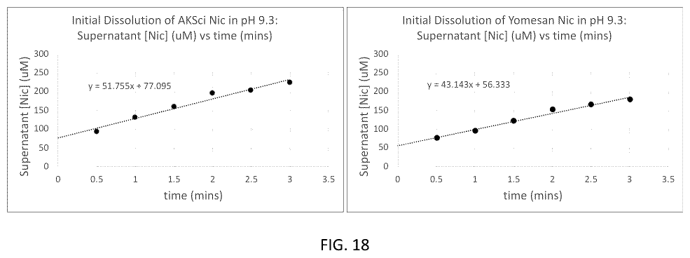

Dissolution models (like Epstein-Plesset [109] that we have used previously to evaluate dissolution of gas [110, 111] and liquid microparticles [112-114]), assume that the concentration at the particle surface is the saturation concentration Cₛ, and that the rate of dissolution (dm/dt) should be proportional to this saturation concentration. Thus, as shown in FIG. 15, when plotted as the normalized concentrations (μM/mg) the initial rates of dissolution are close to being proportional to the equilibrium solubilities, especially for the higher pHs and hence higher niclosamide solubilities from FIG. 11B (where the stirring was standardized). See FIG. 18 for raw data for a comparison of the initial rates of dissolution for AKSci and extraction from crushed Yomesan tablets.

### 4.4. Embodiment 3: Niclosamide Extraction from Yomesan Tablets at pH 9.3 (Optimized Mixing Protocol)

With equilibrium and dissolution rates for the commercial AK Sci niclosamide established at the different pHs and under optimized stirring conditions, the dissolution, and hence extraction of niclosamide from crushed Yomesan tablet material was investigated. 4.5 mg of powdered and ground Yomesan tablets (equivalent to 3.5 mg of niclosamide) was added to 10 mLs of pH 9.34 buffer and stirred for 75 minutes. Measurement at a series of time intervals was again by UV/Vis nanodrop on 2 uL samples taken directly from the stirred suspension. The dissolution curve of supernatant niclosamide concentration [Nic] (uM) versus time (mins) for this ground-up Yomesan powder is shown in FIG. 16. The sample became progressively cloudy as excipient tablet material hydrated, perhaps disintegrated, and was stirred into suspension. Eventually, the measured concentration of niclosamide into solution plateaued, signifying perhaps a limit to the extraction. While the UV/Vis spectrum still retained the double peak profile (333 nm and 377 nm), there could have been some influence of suspended material and so the suspension was filtered through a 0.22 um filter and the clear supernatant was remeasured. As shown, the niclosamide concentration of the filtered sample was still high at this pH 9.34 at 348.3 uM after just 1 hr of stirring. This demonstrates that niclosamide is readily extracted from the powdered Yomesan tablet, including its excipients, in a similar rate and to similar levels as that for pure AK Sci and Sigma niclosamide. Since Yomesan is available world-wide, as are several generics, it would be possible for local suppliers to carry out a similar extraction on the tablets, filter the supernatant (before any equilibration to the lower solubility polymorph) and provide niclosamide solutions to populations in need of the prophylactic and early treatment spray extracted from an already approved and commercially available tablet. Equilibrating 10 mls of buffer with 1 mg of tablet provides this solution of 350 uM niclosamide. Since the tablets contain 500 mg niclosamide, one tablet could, in principle provide 500 mLs of 350 uM niclosamide at pH 9.33 for the throat spray, or 8.75 Liters of 20 uM niclosamide solution at pH 8 for the nasal spray.

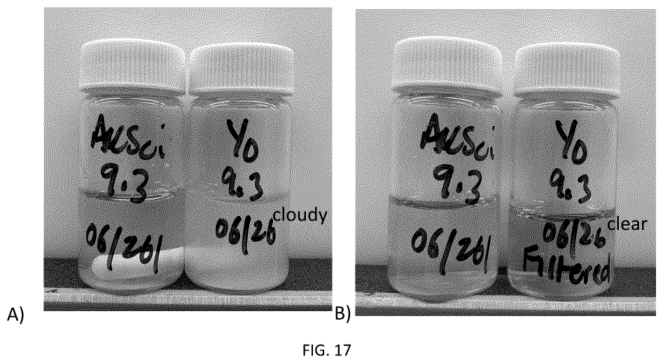

Photographic images of the vials containing the cloudy and filtered samples of the extracted niclosamide suspensions from the ground-up Yomesan tablet material are shown in in FIG. 17 and compared to a solution of AK Sci niclosamide at the same pH.

FIG. 17A shows the cloudy supernatant sample and FIG. 17B shows the clear supernatant solution that was obtained when filtered through a 0.22 um filter to remove the particulate matter that originated from the tablet excipients. If the Yomesan extraction is left stirring overnight, this resulted in a very cloudy suspension and a reduction in the niclosamide supernatant solution concentrations to only ˜109 uM.

Possible explanations for this reduction in supernatant niclosamide concentration with overnight stirring are the reabsorption of niclosamide into hydrating excipients, (such as magnesium stearate), or a conversion of the niclosamide to a lower solubility polymorph, as seen, presented, and discussed later for other niclosamide samples, (such as from Sigma and precipitated from supersaturated solution). In any event, simply filtering out the suspended material, including any tablet-powdered niclosamide that remained still undissolved, maintains the solution concentration at almost 350 uM, and this was stable in solution overnight and for several days.

### 4.4.1. Initial Dissolution of AKSci Niclosamide and Yomesan Niclosamide in pH 9.3 Buffer

For completion, the initial rates of dissolution for AK Sci niclosamide were shown earlier in the comparison of the systems in FIG. 15 as uM/mg. This data is now given as plots of raw data in FIG. 18 in order to compare the rate of dissolution with the ground-up Yomesan tablet powder in uM units, —units that are more conventionally used for efficacy measurements in cell culture. The initial rates of dissolution over the first three minutes are similar between the powdered niclosamide from AK Sci and the crushed and powdered Yomesan tablet. As shown in FIG. 18, the initial rate of extraction of niclosamide from the powdered Yomesan tablet into the supernatant solution is 43.1 uM/min and is only slightly less than the dissolution of powdered AKSci niclosamide at 51.7 uM/min. Assuming the mortar and pestle grinding process created particles of about the same size and hence surface area per gram, these convert to intrinsic dissolution rates of 0.28 ug/s and 0.23 ug/s for AKSci niclosamide and niclosamide from Yomesan powders, respectively. The non-zero intercept appears to come from the initial shaking required to ensure immersion of the poorly wetted niclosamide in the buffer suspension.

Thus, at the intrinsic dissolution rate of 0.23 μM/mg·s, the rate of appearance of niclosamide into the supernatant from crushed Yomesan powder is almost as fast as for pure niclosamide powder from the AK Sci and Sigma product. This suggests that the ground-up powder using the mortar and pestle broke up the tablet material such that niclosamide particles were fairly-accessible to the buffer solution and could readily undergo the dissolution process and hence be easily extracted.

### 4.5. Embodiment 4: Precipitate Niclosamide from Supersaturated Solution and their “Natural” Crystal Morphologies

### 4.5.1. Niclosamide Precipitated from Supersaturated Solution Compared to AK Sci Niclosamide Dissolved from Powder Versus Supernatant pH

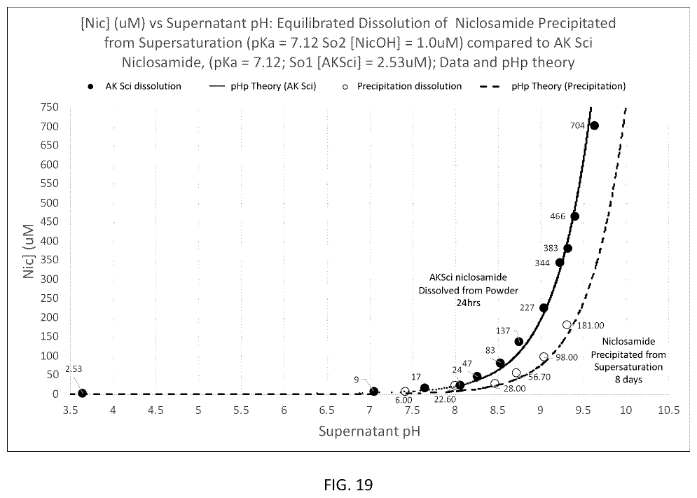

Shown in FIG. 19 in these preliminary experiments, levels of supersaturation were explored and compared to the parent AK Sci material (of unknown processing), including the final equilibrium solubilities of the precipitated niclosamide as a function of the supernatant pH.

As used to accurately make the control solutions, the second way to achieve a solution of niclosamide is to first dissolve the niclosamide in a water-miscible solvent (like ethanol, or acetone, DMSO, or DMA) and exchange the solvent for the aqueous anti-solvent at a final concentration where the niclosamide is still soluble (see above, 3.3.2 Ethanol injection technique). This technique can also be used to create supersaturated solutions from which the niclosamide can (eventually) precipitate and form whatever the stable solid morphology is at that pH. Niclosamide was formed into supersaturated solution at 2-5 times excess niclosamide (with respect to the equilibrium amount of AK Sci niclosamide in solution as in FIG. 11B and Table 1) over the range of pH's and allowed to precipitate over time.

As shown in FIG. 19, the open circles represent niclosamide precipitated from supersaturated solution from the same source (AK Sci). The AK Sci niclosamide was first dissolved into ethanol at 30 mM and then injected into each buffer at 2-5 times the excess concentration of that measured solubility, allowed to precipitate, and fully equilibrate for 8 days and then filtered through a 0.22 um filter prior to taking the clear supernatant for UV/Vis measurement. For comparison, shown in filled circles, is the series of AK Sci niclosamide dissolved from ground-powder, as given earlier in FIG. 11B.

What this data shows is that niclosamide, when precipitated from supersaturated solution, equilibrates to a much lower final supernatant niclosamide concentration than the parent compound, which is presumably reflective of the solubility of the solid form at each pH. Prior to precipitation, it was possible to achieve supernatant concentrations (kinetic solubility) as high as 3 mM niclosamide at the highest pH of 9.5, —a supersaturation of ˜3 mM/200 μM=15 times. The data shows that, while the AK Sci niclosamide powder was relatively stable in solution at the high concentrations achieved, precipitation from supersaturated solution forms the lower solubility polymorph at each pH, that still does have a pH dependence for its now thermodynamic solubility.

It is instructive to also compare the data to pHp theory using the same pKₐ and the measured intrinsic solubilities for each sample. The intrinsic solubility of the AKSci niclosamide (Sₒ1) was measured to be 2.53 uM±1.0 uM (solid line through the filled circles) and the intrinsic solubility of the low solubility niclosamide polymorph (Sₒ2) was measured to be 1 uM±0.26 uM (dashed line through the filled triangles). Again, the pKₐ used in the pHp theory for both curves is the same value of 7.12 that was derived from fitting the AK Sci dissolution data in FIGS. 11A and B. Since the pKₐ is the equilibrium balance between the acid and salt forms in solution, it is satisfying that this same pKₐ of 7.12 can be used to fit both sets of data. As mentioned earlier, there was some difficulty in measuring these low pH solubilities, but there was a discernable difference between instrument noise for the blank and the sample.

### 4.5.2. Supersaturated Solutions Form a Series of Niclosamide Morphologies of Reduced Solubility

Corresponding to the supernatant concentrations given in FIG. 19, samples of the precipitated material were taken and viewed under the optical microscope. As shown in FIG. 20, the photographic images of precipitated particles from supersaturated solution at each supernatant pH are full screen images using a 40× bright field objective.

At pH 7, where the low solubility protonated acid makes up almost 50% of the niclosamide in solution (see Henderson Hasslebalch curve in FIG. 2), niclosamide precipitates as flat sheets formed in the stirred aqueous buffer that make a mass of gel-like particles in suspension. Macroscopically, these are the characteristic “fluffy” white particles seen swirling in the vial (see image in FIG. 5). This flat sheet, particulate, gel-like morphology persists at pH 7.5, 8.0, 8.25 and 8.5. Being formed from a 1:99 dilution of ethanolic niclosamide, that is exchanged for the excess pH buffer under rapid stirring, some sheets are observed to fold, as in the image for pH 8.25. Then, at pH 8.75, the morphology makes a transition to the more needle-like polymorph that is also observed for the precipitated particles at pH 9.0 and 9.3, where the deprotonated niclosamide salt is the dominant species and is characteristic of monohydrate.

What these microscopic images demonstrate then, seemingly for the first time in the literature, is that these most stable niclosamide hydrates [17] not only have a pH dependence to their solubility but also have a pH dependent morphology that accompanies their changing solubility as a function of pH.

### 4.5.3. Niclosamide Gel-Like Particles Also Display a Strong Hydrophobicity and Coat Gas Bubbles

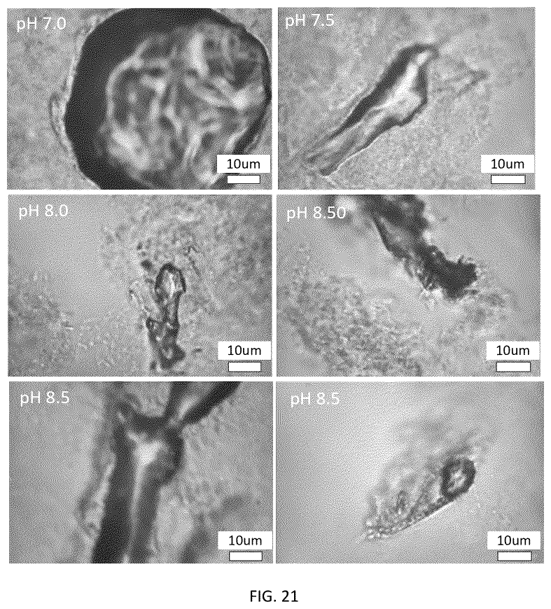

A new and interesting, but not unexpected observation, for such a hydrophobic drug, was that the niclosamide gel-like particles also display a strong hydrophobicity. In some images of the precipitated samples, dark objects were often observed. As shown in FIG. 21, what these represent are gas microbubbles that follow the crumpled contours of the niclosamide precipitated sheet formed at pHs 7.0, 7.5, 8.0 and 8.5 as the precipitate adheres to the bubble surface. Because the particles are formed by precipitation in a rapidly stirred buffer environment, it seems that gas microbubbles can get trapped.

These characteristically optically black masses of air show a complete lack of surface tension (or tension in the surface). That is, rather than being round and exhibiting their usual air-water surface tension and concomitant Laplace pressure, they are deformed to the shape of the niclosamide material. Clearly, as one would expect from niclosamide's low solubility and moderate log P of 4, at these pHs, the niclosamide particles are quite hydrophobic. This was also borne out by the way the particles of powder when added to a vial for the dissolution test would float at the air solution interface unless well shaken to wet the powdered particles and immerse them in buffer suspension.

This kind of zero tension and zero Laplace pressure has actually been observed and well-characterized before for lipid coated gas microbubbles (as solid lipid monolayers) in our micropipette experiments that measure interfacial tensions as well as gas bubble dissolution in undersaturated solutions [110, 115, 116]. Here though, the hydrophobic precipitated niclosamide sheets form a kind of Pickering emulsion that is stabilized by the sheet-like particles adsorbed onto the interface between the aqueous and gas phases.

### 4.4 Embodiment 5: Dissolution of Niclosamide as a Water-Precipitate, and as Recrystallized Material from Acetone and Ethanol

### 4.4.1. Dissolution Profiles

Given the high solubilities observed by the AK Sci material, while other sources (Sigma) and precipitated material readily converted to the less soluble and more stable hydrate polymorphs, preliminary studies were conducted to create different precipitates and cosolvates in order to evaluate their dissolution and crystal morphologies. These studies were conducted to initially compare the samples with the AKSci material (and try to ascertain if it was a water, ethanol or acetone solvate; it wasn't).

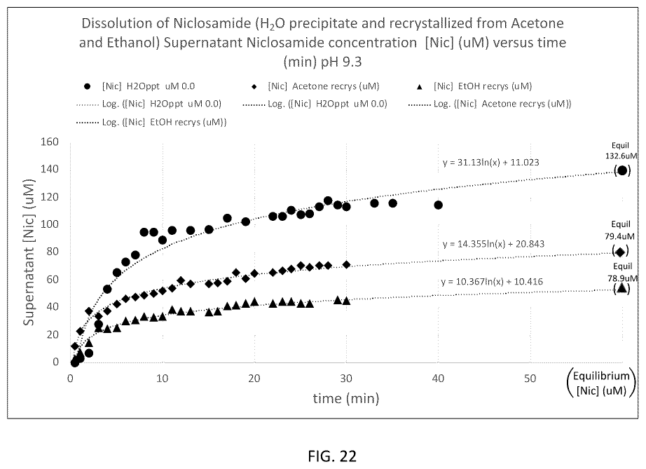

Samples of niclosamide were made from the AK Sci original material as a water-precipitate and recrystallized from Acetone and Ethanol, as described in methods. The same mass of ground material, 3.5 mgs (˜1 mM equivalent), was added to each 10 mls of pH 9.3 buffer in a 20 mL scintillation vial and stirred with magnetic stir bar. 2 uL samples were taken at time intervals and their absorbance measured on a UV/VIS nanodrop spectrophotometer at 333 nm. The concentrations of niclosamide (uM) in the supernatant versus time (mins) are plotted in FIG. 22 for the water precipitate, and the recrystallized niclosamide from acetone and ethanol.

Dissolution profiles were therefore determined for each of the dried samples (water precipitated, and acetone and ethanol recrystallized) as well as the equilibrium supernatant concentrations several days after dissolution, all determined at pH 9.3. As can be seen, the dissolution rates go as Nic_{H20ppt}>Nic_{Acetone recrys}>Nic_{EtOH recrys}, although the initial rates measured as the intrinsic parameter uM/mg of material were: Nic_{H20ppt} (0.104 uM/mg) >Nic_{EtOH-recrys}, (0.0439 uM/mg) ˜Nic_{Acetone-recrys} (0.0369 uM/mg).

This data shows that the commercial niclosamide product from AK Sci was likely not recrystallized from acetone (as we were told) because the as-received AK Sci niclosamide has a much higher solubility (˜383 uM) at pH 9.3 than the acetone recrystallized material (79.4 uM). It also shows how the water solvate that was dried and then re dissolved at pH 9.3 has a similar (132.6 uM) solubility to that precipitated in situ as in FIG. 19 of 180.9 uM. Also, the acetone and ethanol cosolvates have an even lower solubility than the water precipitated material.

### 4.4.2. Crystal Morphologies

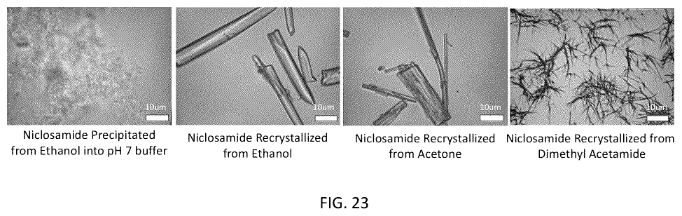

Images of the morphologies of the water-precipitated and acetone- and ethanol-recrystallized samples are shown in FIG. 23. As shown before, and included here for comparison, niclosamide precipitated from supersaturated solution (after solvent exchange from an ethanolic solution into excess water at 1% ethanol) has a very different morphology to the rod-like, needle-like, samples recrystallized from acetone and ethanol. These latter structures are more reminiscent of the methanol and other cosolvates made and characterized by van Tonder et al [5, 17, 88, 89].

These images taken of the various samples illustrate that the source of niclosamide, including its synthesis and post processing solvent recrystallization or precipitation recovery, can dramatically influence the final intrinsic solubility of the drug compound and the amount that can be dissolved in aqueous solution at any pH. This, in turn, is expected to influence the pharmaceutical performance of the drug product, especially its solubility limit and dissolution, both of which affect its ultimate bioavailability. This is especially important in our (and other's) applications. As mentioned above and discussed further below (in section 5.2), the most effective way for niclosamide to permeate through the mucin layer that normally covers and protects the underlying nasal and buccal epithelial cells is as a soluble molecule. Also, any suspension of microparticle material is expected to undergo a conversion to the more stable and least soluble monohydrate form. However, if the solid is filtered out or removed by centrifugation, the remaining supernatant retains its high soluble form, unless heterogeneous nucleation might trigger the monohydrate.

### 4.5. Embodiment 6: Sigma Niclosamide Dissolution and Overnight Stirring Also Produces Lower Solubility Material

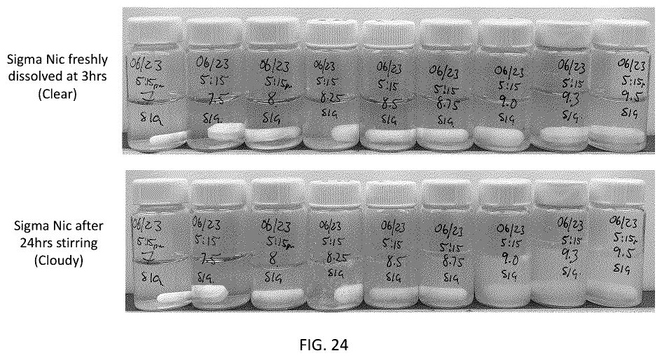

As shown above (FIG. 13), the Sigma Niclosamide product readily dissolves and, over a period of 3 hrs, it achieves a niclosamide supernatant concentration of 680 μM. This was actually slightly more soluble than the AKSci material (AK Sci was 564 uM) at this pH. However, while the AK Sci material appeared to be generally stable as excess powdered particles that had somewhat dissolved and were in equilibrium with their supernatant solution over the short term (3 hrs), the Sigma material converted to a much lower solubility polymorph over a period of the next several hours. 24 hrs after initially starting the dissolution experiment, as shown in FIG. 24, the supernatant went from a clear solution (top image) to a cloudy suspension (lower image).

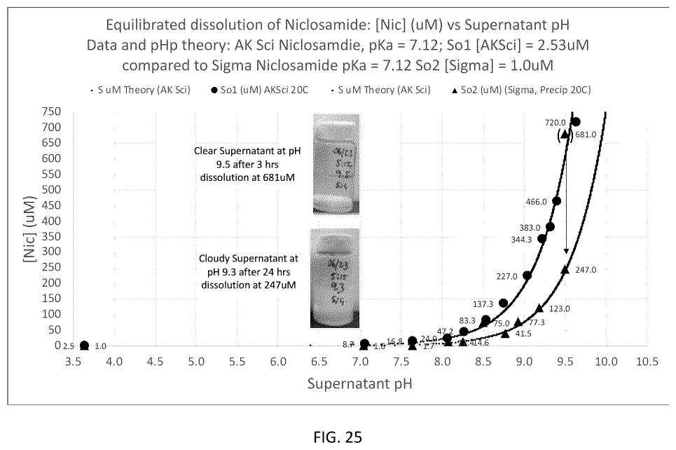

As shown in FIG. 25, when the supernatants were filtered through 0.22 um filters and the supernatant niclosamide was measured by UV/Vis spectroscopy, the supernatant niclosamide concentration had fallen from 681 uM to 247 uM at ˜pH 9.5, —a loss of material concentration of 434 uM. Thus, when dissolved and equilibrated over the range of pHs 7 to 9.5, the final supernatant concentrations were in a similar range to those measured for the niclosamide precipitated from supersaturated solution (in FIG. 19), i.e., they both showed solubilities (Sigma Nic at pH 9.3=103 uM; precipitated material=180.9 uM) consistent with forming the more stable hydrated polymorph that nevertheless still showed a pH dependence to the amount in supernatant solution.

The experimental data is compared to the pHp model using the measured value of intrinsic solubility for the low solubility polymorph (designated Sₒ2) of ˜1.0 uM and the same derived pKa of 7.12 for the fit. This overnight equilibration and resulting decrease in supernatant concentration produced a change in the particle morphology. Observing these samples solved the mystery of why the Sigma niclosamide showed such different behavior to the AKSci supplied material. Herein lies a cautionary tale for any researcher who obtains niclosamide from a given supplier, since the equilibrium morphology that can readily be obtained could be the low solubility monohydrate polymorph.

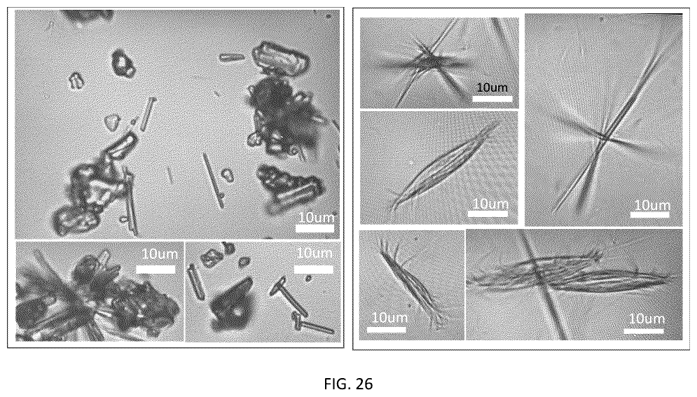

As shown in FIG. 26 the original Sigma niclosamide material displayed a mixture of block- and rod-like particles and after overnight equilibration with the excess material these were converted to long, more fibrous, and spiky bundled structures with a background of smaller fainter fibers. This material had a much lower intrinsic solubility (1 uM) than the parent Sigma Niclosamide powder and a reduced amount of niclosamide in supernatant solution of ˜3.5× over the whole pH range.

To investigate this further and compare with the AKSci material, additional optical microscope images were of excess niclosamide powder equilibrated in buffers across the whole pH range. These are shown in FIGS. 27A and B and explain the difference in dissolution and equilibration behavior between the two supplied Niclosamides. That is, the difference between the Sigma niclosamide product and the AK Sci product is that, while both have an appearance of block like crystals, the Sigma material does already have rods and spikes indicating that it contains a mixture of polymorphic niclosamide including the low solubility monohydrate, and so were likely processed after synthesis by recrystallization or precipitation in different media. Also, the powdered material is more prone to conversion to the more stable (apparent) monohydrate after overnight equilibration in the pH 9.3 buffer than the AK Sci material that only contains block-like morphology and a complete absence of the spiky monohydrate morphology.

### 5. Discussion of Niclosamide Solution Embodiments

Discussed first are the main experimental results associated with the equilibrium and kinetic dissolution experiments for niclosamide into pH buffers from a commercial source and how this enables making the niclosamide-based nasal and throat sprays. Next, is addressed the extraction of niclosamide from approved Yomesan tablets and how this might expedite regulatory approval, especially outside the USA where it is widely approved. This is followed by further scientific findings concerning the reversion of niclosamide from other, more soluble, forms of niclosamide when left in suspensions to more stable and lower solubility polymorphs when supernatants are left in contact with the excess solid material. Filtering out the excess niclosamide preserves the achieved amount in solution unless heterogeneous nucleation eventually precipitates the more stable polymorph and then the supernatant concentration can decrease.

Attention is then turned to a discussion of what is known about epithelial tissue kinetics and viral transport, replication, and secretion of virions from the host cells and how this could impact formulation development and treatment options as presented above. Finally, based on the results, thinking, analyses, and conclusions laid out here, the discussion ends with a cautionary tale for any microparticle material, especially for optimized local delivery to nasal and respiratory epithelia. Because it too is a nasal spray, particular discussion is focused on the newly formulated micronized niclosamide embedded in spray dried lysozyme [91] and how it could be more fully optimized for nasal and inhalation administration. That is, because of the low solubility of niclosamide per se and the fact that the protective mucin that covers the epithelial cells is a barrier to such microparticles, the only possible transport of niclosamide to the underlying layer of nasal and respiratory epithelial cells is the niclosamide that is in molecular solution. Being dissolved in simple pH buffers, our niclosamide solutions are also much easier to manufacture than having to micronize the niclosamide, and then spray dry it with microparticles of lysozyme protein.

### 5.1. Niclosamide Solutions can Readily be Obtained by Dissolving Niclosamide Powder into Simple Buffer

The main result from this work according to the data for the AK Sci (brick-like) polymorph and the pHp theory that is fitted through the data, as exemplified in FIGS. 11A and 11B, is that a 20 uM niclosamide solution can readily be obtained by dissolving niclosamide powder into aqueous solution at a pH of 7.98; a 200 uM solution of niclosamide can be obtained in buffered solution at pH 8.92; and the concentration can even be raised to 300 uM at pH 9.10. The ultimate solubility at pH 9.63 was on the order of 700 uM. The preferred way to make the solutions though is by the solvent exchange technique that makes niclosamide solutions that are not in contact or deleterious equilibration with undissolved excess materials.

For the nasal epithelium, while the average pH in the anterior (front) of the nose is 6.40 and the average pH in the posterior (back) of the nasal cavity is 6.27, the overall range in pH is 5.17-8.13 for anterior pH and 5.20-8.00 for posterior pH [108]. Therefore, as shown for the AK Sci (brick-like) polymorph and even the mixed polymorphic Sigma sample over a short 3 hr equilibration time, a 20 uM niclosamide solution is 20× that required to completely prevent viral replication in SARS-COV-2 infected cells as measured for niclosamide by Jeon [14] of 1 uM in Vero 6 cells, and more recently by Brunaugh et al [91] of 0.4 uM, including a purported enhancement by lysozyme in culture, also in Vero 6 cells, (i.e., not mucin covered cells). For what could be considered the more relevant Calu-3 lung cells (still not mucin covered) where the IC₁₀₀ was ˜2 uM-3 uM, the available concentrations for the AK Sci polymorph niclosamide solutions made here are still 10× the efficacy. If testing in animals and humans reveals 20 uM is safe, dose escalation is possible since as shown again by the data and pHp theory in FIG. 11B, the niclosamide concentration can be increased to 30 uM at pH 8.16 and 50 uM is obtained at only pH 8.39. For throat spray early treatment, concentrations can be escalated up to 300 uM at an orally/respiratory tolerated pH of 9.19. Thus, having obtained the intrinsic solubility of this polymorph of niclosamide (2.53 uM), shown how the amount in solution increases with increasing pH and then obtaining the appropriate fit to the data with pHp theory by choosing a pKa of 7.12, the utility of this data and the pHp model is that it can predict the amount of niclosamide in solution at any pH.

### 5.2. Niclosamide can be Readily Extracted into Buffer from Yomesan Tablets and Other Generics

The other major result of this study is that Niclosamide can be readily extracted into buffer from crushed and ground Yomesan tablets where the niclosamide is available to dissolve in the buffer at a similar rate to pure AK Sci niclosamide powder, achieving 350 uM within ˜1 hr). The initial rates were 51 uM/min for AK Sci niclosamide and 43 uM/min for niclosamide extraction form Yomesan, that convert to 0.28 ug/s and 0.23 ug/s respectively. The dissolution experiment in FIG. 16 nicely showed that ground up Yomesan powder can be dissolved into pH 9.34 buffer to achieve a final niclosamide concentration of the filtered sample of 348.3 uM. This is more than enough to use as a 300 uM throat spray and can be easily diluted into pH 8 buffer to give the 20 uM nasal spray. This recognition now motivates the reformulation of the already used and approved tablets into a sprayable suspension. This new observation opens the door for a different route to making the niclosamide nasal and throat sprays from already regulatory approved and commercially available tablets (including a series of generics). This could also have implications in expediting the regulatory pathway especially in Europe and elsewhere, where the tablets are still under regulatory approval.

### 5.3. Niclosamide can Readily Revert to its Most Stable Form, and so Reduce its Solubility

In support of these two major take a-ways, are the studies that explored the low solubility apparent monohydrate polymorphs. That is:
For one commercially available niclosamide powder (AK Sci), after dissolution equilibrium was attained over a period of a few days at pH 9.3, the high concentration solution supernatant was stable despite being in contact with its original excess powder. Upon microscopic observation the excess undissolved material retained its original brick-like morphologic appearance signifying that there was no conversion.
For the Yomesan tablet powder, it reverted to a lower amount of niclosamide in supernatant solution that could have been an interactive uptake by hydrating excipients in the excess tablet material or, more likely, it reverted to a lower solubility polymorph than was used to make the tablets in the first place.
For the Sigma niclosamide, its initially dissolved, high concentration solution was stable for a few hours, but when also in contact with its original excess powder, converted to a lower solubility polymorph. Here, microscopic examination of the original material showed that it contained a mixture of a brick like morphology and a spiky needle shaped morphology, the latter being characteristic of the low solubility monohydrate polymorph. Thus, the Sigma material was already predisposed to conversion to the low solubility polymorph and did so upon overnight equilibration with the excess material acting as a heterogeneous template.
For niclosamide precipitated from supersaturation, the precipitate eventually achieved their own low solubility solid forms, and also displayed the needle-shaped, spiky morphology
For the water-precipitate, and acetone and ethanol cosolvates, the solubilities were similarly low across the whole pH range and again displayed the needle-shaped, spiky morphology.

Why all this intrinsic solubility, amount in solution and the appearance of low solubility polymorphs is important to formulation design and performance is nicely summarized in a quote from van Tonder et al [5], “Hydration of niclosamide crystals can also be detrimental to the performance of niclosamide powder during various pharmaceutical processes because this drug can be exposed, potentially, to water during crystallization, wet granulation, lyophilization, spray drying, and aqueous film coating. Water can also be taken up during storage in an environment containing water vapor or from excipients that contain water and are capable of transferring it to other ingredients. When these niclosamide hydrates are formed, water is incorporated into the crystal lattice”.

A summary of the data is now shown in FIG. 27. Included are the data from FIG. 19 (Niclosamide Precipitated from Supersaturated Solution) and FIG. 25 (Sigma Niclosamide after 24 hrs) all combined with the equilibrium amounts of niclosamide in buffered solution measured for the water, acetone and ethanol solvates (FIG. 22), and compared to the original AK Sci material (from FIG. 11B). The experimental data is also compared with the two pHp curves, one for the AKSci polymorph with an intrinsic solubility of 2.53 uM and the other for the low solubility (presumed) monohydrate polymorph with a measured solubility of 1 uM, and both curves using the same pKa for niclosamide in solution of 7.12.

Thus, as shown in the optical microscope images in FIG. 26, and FIG. 27, the (probably not surprising) conclusion is that niclosamide can readily revert to its most stable polymorphs (—the monohydrates and cosolvates identified by van Tonder et al [5, 17]) that have a lower intrinsic solubility of niclosamide in aqueous solution when in contact with these solid forms and this translates to a lower amount in solution across the whole pH range.

While still showing the expected pH dependence for solubility i.e., dependent on the acid and salt solution equilibrium, there is a clear distinction and drop in solubility, dependent on the solid form in equilibrium with the species in solution. What this shows is that for both the Sigma niclosamide and the precipitated materials, as well as the cosolvates, the amount of niclosamide in actual solution is dependent on the nature of the solid form it is in equilibrium with. Unfortunately for the commercial powders, this information is not available because the manufacturers will not divulge their post synthesis processing to recover the niclosamide material as it is a trade secret.

While beyond the scope of this current study, further studies by Raman and x-ray diffraction FT-IR/Raman, and calorimetry could be carried out and are being planned to gain more insight into the crystal forms of these commercial products and the precipitates and solvates.

Thus, when particulate niclosamide is in solution, an equilibrium is set up between the energetics of the solid form (bonding within the crystal form, e.g., crystal lattice energy) and the species in solution. To reiterate, as reported by van Tonder, the most stable forms of niclosamide are the monohydrates that have the lowest solubility compared to anhydrates and other cosolvates. But these forms are not necessarily what are purchased from niclosamide suppliers at AKSci, Sigma or, as with Brunaugh et al [91], from a supplier, Shenzhen Neconn Pharmtechs Ltd. (Shenzhen, China), that is also of undefined crystal morphology, and hence solubility, and has not been measured as a function of pH.

As is well known in pharmaceutics, the solubility and permeability behavior of drugs play a major role in bioavailability. This is particularly important in local delivery via any nasal and throat spray or nebulized lung inhalant, since it is only the soluble form that can permeate the mucin layers and reach the epithelia. The value for that solubility is therefore critical for effective drug delivery. In fact, apart from ensuring direct access through the mucin, it was this recognition (of such a low ˜2-3 μM solubility for niclosamide at neutral pH), that prompted the invention and development of a niclosamide solution as opposed to any microparticle formulation that were all explored in the work that went into the series of provisional patents [16].

### 5.4. In Summary: “Niclosamide is not Niclosamide is not Niclosamide”

To summarize what has been learned is that, when niclosamide is present as a dissolved molecule in aqueous solution, its behavior is predictable by concepts associated with drug protonation, and deprotonation embodied in the Henderson Hasselbalch and precipitation pH theories. These theories bring together the pKa of the niclosamide with the intrinsic solubilities of its dissolving polymorphs. Because it has a reported pKa of around 6 to 7 [22, 82, 84, 85], measured to be 7.12 in this work, the theoretical maximum amount in solution is determined by the relative amounts of its pH-dependent protonated and unprotonated forms. As taught, and shown here, the solubility of niclosamide follows the Henderson-Hasselbalch and precipitation-pH (pHp) models (FIG. 2). Basically, the amount of niclosamide in aqueous solution increases at the higher pHs because the fractional amount of the lower soluble acid form decreases, and the more soluble deprotonated salt form is the dominant species. This pH-dependent behavior provides a mechanism for increasing the availability of niclosamide in simple solutions to provide optimized formulations as nasal and throat sprays. Such formulations can be readily prepared simply by dissolving niclosamide powder into buffered aqueous solution, or preferably made by solvent exchange from concentrated ethanolic niclosamide into pH buffers. Also as shown here for the first time, it is possible to extract niclosamide into buffered solution from commercially available and regulatory approved anti-helminthic tablets that are normally used for worms [8], including generics.

However, if and when precipitated or present as a particulate material, micronized or surfactant- or protein- or polymer-“stabilized” microparticles, the amount of niclosamide in aqueous solution is determined by the nature of the crystal or amorphous material that the local aqueous solution is in equilibrium with, or is moving towards. This is where careful preformulation drug characterization is needed in order to fully understand and predict the nature of niclosamide's morphological forms, its resulting solubility in aqueous media, and hence its bioavailability in (any) dosage form.

Presented here then were preformulation characterizations, conducted experiments, results, comparisons with theory and discussion that spoke directly to a series of issues in optimizing nasal and throat sprays of niclosamide:
The data characterized the equilibrium and kinetic dissolution behavior of niclosamide from dry powder including niclosamide from various suppliers and co-solvates showing that the AK Sci polymorph (brick-like morphology) provided stable supernatant solutions even when in equilibrium with its original excess powdered material.
Niclosamide from a different supplier, (Sigma) contained a mixture of polymorphs including both the brick-like and spiky needle-shaped morphology characteristic of the low solubility monohydrate. Consequently, upon dissolution of excess powdered material the brick-like component readily dissolved in a similar manner to the AK Sci material, but after 24 hrs equilibration the excess material had all converted over to the low solubility polymorph and the amount of niclosamide in pH-buffered solution had concomitantly been reduced by about a factor of 3 across the whole pH range.
The data also evaluated niclosamide's precipitation from supersaturation in aqueous buffered solutions and showed that niclosamide could readily revert to its most stable and lowest solubility hydrated form upon such precipitation.
An optimized buffered aqueous solution of niclosamide was provided that is assured to permeate through the protective mucin layers that otherwise protect respiratory airway epithelial cells from microparticles, including ones that are formulated as such in the more traditional nasal spray formulations.
A protocol was provided that can be used to extract niclosamide into buffered solution from commercially available and regulatory approved niclosamide tablets, including Bayer's Yomesan and other generics that are available worldwide.

### 6. Why Niclosamide? the Nasal Spray Formulation, its Target Mucosal Epithelial Cells and its Action as a Virostatic Anti-Viral for SARS-COV2

Attention is turned now to answer the question, “Why niclosamide?” What is it about the way niclosamide acts in the host cell that makes it such a good candidate for a prophylactic nasal spray and early treatment throat spray to combat the SARS-COV-2 virus, its more infective variants, and other respiratory viruses?

Consider the kinetics of each aspect of this drug delivery system, i.e., the kinetics of the target tissue, —the mucin barrier and underlying epithelial cells; the kinetics of the virus and its stages of infection; the kinetic transport of molecular niclosamide from solution through the mucin; and the virostatic effects of niclosamide on the host cell that inhibits three of those stages of infection. Together, they all have consequences for how prophylaxis and early treatment may be made most effective in terms of the physiology and macro-pharmacology of viral infection and what a safe and effective drug delivery system for the naso and oropharynx might look like, clinically (in section 7). All these analyses have revealed a cautionary tale for any microparticle material that seeks to deliver drugs, especially low solubility ones like niclosamide, to cells underlying a nasal and upper respiratory mucosa. Finally, as presented in section 7, this all leads to suggestions for future work and a series of hypotheses yet to be tested at the cell and preclinical animal level, and on to regulatory issues and clinical testing.

### 6.1. The Six Stages of Viral Infection at the Host Cell and how Niclosamide Inhibits Three of them

The virus first infects the nasal epithelium and viral load is highest in pharyngeal secretions early in the course of infection [117]. Nasal epithelial cells were shown to express the highest levels of angiotensin-converting enzyme 2 (ACE2) and of the cellular serine protease TMPRSS2, both working as the main entry receptors for SARS-CoV-2 following interaction with its viral (S)pike protein [118]. According to Goulding [4], the series of infective events are attachment, penetration, uncoating, replication, assembly, and virion release. As reported by Harcourt et al, this whole process can take ˜36 hrs [6].

### 6.1.1 Kinetics of the Virus and Stages of Viral Infection

Without getting into too many details of the molecular biology and biochemistry (there are many papers now in the literature that speak to all of this in a qualitative sense), the goal here is to delineate the sequence kinetically and illustrate where niclosamide can have its action.

First, FIG. 29 shows the viral infection sequence from contacting the nasal mucin to secretion of active virions. The top image is taken and adapted from Hou et al, [1] shows SARS-Cov-2-infected ciliated cells in the first layer of the epithelium from a COVID-19 patient's bronchi. To get this spectacular image [1], the trachea from a SARS-CoV-2 autopsy was probed for SARS-CoV-2 by RNA-ISH—a combination of RNA and In Situ Hybridization techniques. Colorimetric detection of SARS-CoV-2 demonstrates infection of the surface epithelium. Shown in the image is the co-localization of SARS-CoV-2 (originally red) but now greyish highlights on the ciliated cell surfaces with cell-type-specific markers (originally green) determined by the dual-immunofluorescent staining (acetylated α-tubulin cilia marker). This data from Hou et al shows that the virus seems to preferentially infect the ciliated cells and not the nasal submucosal glands [1]. Other data by Zhu et al, [61] indicates that virus can be found at the apical surface of both ciliated cells and secretory cells, plus, the observation of inclusion bodies formed by viral components in the cytoplasm, and therefore potential infection of both cell types.

In any event, the virus appears to just infect the top layer of ciliated cells within the superficial epithelia lining-proximal airway surfaces, particularly in the trachea, but may also infect secretory cells [61]. Above this superficial layer of cells is a mucin layer depicted as a speckled pattern. FIG. 29 schematically shows that it is ˜15 μm thick, and so comparable in depth to the first later of the epithelium.

How long might it take for transport of the virus through the mucin to the epithelial cells? The SARS-COV-2 virus particle is measured to be 80 nm-120 nm in diameter as detected in electron-microscopic examination of cell culture supernatant fluid [119]. A Stokes-Einstein-equation calculation gives this 80 nm-120 nm particle a diffusion coefficient in water of 5.4×10⁻⁸ cm²/s to 3.6 ×10⁻⁸ cm²/s respectively. Olmsted found that similarly small virus-like particles, (i.e., the 38 nm diameter Norwalk and 55 nm diameter HPV particles), surprisingly, diffused in mucus as fast as they diffused in saline buffer. Thus, if the 80 nm-120 nm diameter corona virus also does not interact with the mucus, it would pass through a hydrated 15 μm thick mucin layer by diffusion in 8-12 mins, ready to bind to the cell receptors.

What happens next is also shown in the lower schematic in FIG. 29 adapted from Docea et al, [2].

As discussed by Belouzard et al [120], following 1. Attachment by binding to the ACE2 receptor, enveloped virus entry can occur directly at the cell surface or after internalization via endocytosis. If they transition to endosomes then, 2. Penetration via endosome uptake could take from seconds to minutes. In order for the viral RNA to get into the cell, there has to be 3. Uncoating of the virus, i.e., a fusion of the viral membrane with host membrane to release its RNA into the cell cytoplasm. This is driven by large conformational changes of the spike protein initiated by receptor binding itself, but may need additional triggers such as pH acidification, as occurs naturally in the endosome. The choice of entry mechanism (surface plasma membrane or endosome) may depend on the cell type, hence the need to carry out viral infection studies not on Vero 6 cells, or even Calu-3 lung (cancer-derived) cells that do not have ACE-2 receptors, but on the airway epithelial cells the virus actually infects, as we have started to do [45].

Once in the cytoplasm, 4. Replication begins via transcription-translation of the viral RNA. This is called the Eclipse period and is the time between viral entry and appearance of intracellular virions. As reported by Harcourt et al, this can take ˜24 hrs [6] and it requires ATP from the mitochondria. 5. Assembly of the virus occurs in the Golgi and can take another ˜12 hrs. The assembled, complete, and functional virions are prepared for 6. Secretion out of the cell. Thus, the time between viral entry and appearance of extracellular virions, the so called Latent period is, in total, about 36 hrs [6] but could be longer up to 48 hrs-72 hrs [61]. The virus particles are therefore secreted into the mucus and from there can reinfect other parts of the epithelium or be carried by the mucus into the lungs. An interesting observation from high resolution cellular imaging by Zhu et al [61] showed the aggregation of organelles close to the apical surface, including mitochondria, vesicles, virus particles, and viral inclusion bodies. Interestingly, virus particles were not enclosed within a binding membrane but adjacent to the mitochondria. This is interesting because, as described next, niclosamide slows or inhibits mitochondrial function.

### 6.1.2. The Virostatic Effects of Niclosamide on the Stages of Infection

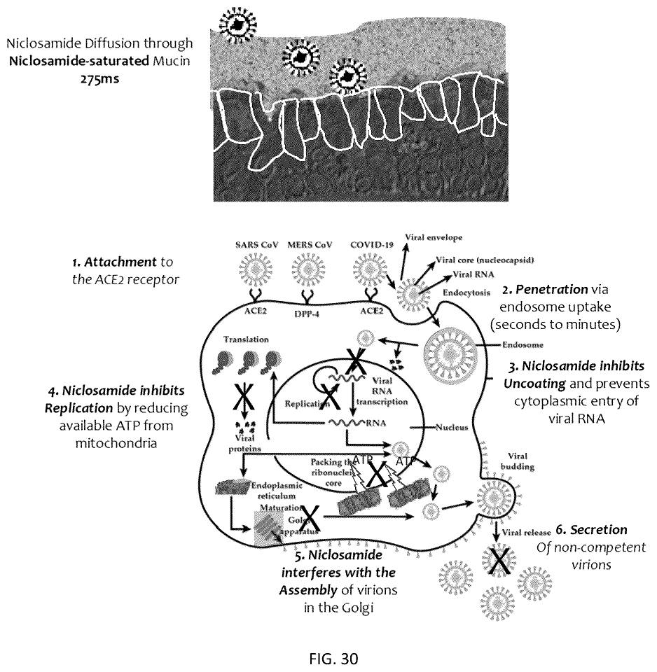

The three stages of viral infection that niclosamide can have a direct effect on are shown in FIG. 30. The top image is now modified schematically to indicate the saturation of the mucin with the concentrated niclosamide solution, shown in more intense grey speckling. The white outlines of the cell membranes are an attempt to show schematically the incorporation of niclosamide into the plasma membranes of at least the top layer of epithelial cells. Most importantly, it is expected that niclosamide can enter the internal organelles of the endosome, lysozome, mitochondria, and Golgi, but this is not depicted here, although it is implied.

Although not shown because of the lack of color, niclosamide saturates interior organelle membranes as well, including the endosome, mitochondria, and Golgi. Thus, as shown in FIG. 30, once it gains access to the nasal mucosa, the virus proceeds as normal with stages 1. Attachment and, assuming it is taken up in endosomes, 2. Penetration. Once in the endosome, as the cell proceeds to reduce the endosome pH, the next stage is that it 3. Uncoats, i.e., it fuses with the membrane, and the RNA gains access to the cell cytoplasm. However, once in the niclosamide-saturated endosome, prophylactic niclosamide in that membrane would dissipate the pH gradient and not allow the normal acidification, and so prevent the ensuing conformational change to the spike protein. Thus, at this stage, niclosamide inhibits 3. Uncoating and prevents cytoplasmic entry of viral RNA. If any viruses escape this stage or infection has already occurred and cells already contain virus, they move to 4. Replication. It is here that prophylactic niclosamide, or niclosamide administered to nasal and throat cells in an early treatment option, has its major effect of reducing the ATP in the cell. As mentioned earlier, niclosamide dissipates the pH gradient across the inner mitochondrial membrane that is necessary to generate the ATP via the proton-driven ATP synthase in that membrane. In the Jeon study [14], the IC₅₀ for inhibiting viral replication in Vero 6 cells was 0.28 uM, and the replication was completely inhibited at 1 uM niclosamide, In the subsequent work by Ko et al in Calu-3 lung cells [34] it's IC₅₀ was 0.84 uM and inhibition of replication was at ˜2 uM-3 uM.

The preliminary studies by Kim [45] in airway epithelial cells, mentioned above and briefly reiterated here, show that the IC₅₀ for cell viability and hence ATP production is effectively reduced at 20 uM-30 uM niclosamide. Thus, a 20 uM-30 uM niclosamide solution at only pH 7.98 provides the necessary reductions in ATP production that reduces cell viability (as needed to stop viral replication). Importantly, the cells were 100% living and 100% recoverable when evaluated 18 hrs later after niclosamide wash out, and so were not lethally impacted by the presence of niclosamide, just “put to sleep”. Comparing this data by Kim to that by Ko et al [34] shows that a 50% reduction in ATP in the host cell is 10 to 20× that required to completely inhibit viral replication, and does not kill the host epithelial cells. This is the basis for the prophylactic. Thus, at this stage, niclosamide inhibits 4. Replication by reducing the host cell ATP available to the virus for that process. Basically it “turns down the dimmer switch” on the cell's energy supply so that the virus cannot use the cell's own machinery to replicate; the virus itself is put in lockdown.

It is also interesting to consider what happens at higher niclosamide concentrations that can be achieved at the higher pH of only 9.1 and used in the throat. Again referring to the Kim study, [45], incubation with 300 uM Niclosamide dropped the cell viability, and ATP content, to 30%. Upon washout of the drug, only 17% of the airway epithelial cells were registered as still alive, and so 83% of epithelial cells did not survive and may have undergone apoptosis. Why this is interesting is that, as mentioned above, once replicated in the nose, virus particles and any infected and shed cells would travel down the back of the throat towards the lungs where they have their devastating effects. As delineated below (5.2.5 Timeline), if these shed cells are actually still alive and incubate the virus, then it would be ideal to make sure they too have their ATP reduced or are actually killed by niclosamide at these higher concentrations, and so completely eliminate any viral replication as they move into the lungs. Given that this epithelium is a regenerating tissue, a course of niclosamide spray could in effect stop the virus in the shed cells such that the epithelium regenerates and repairs itself [121]. Of course, this is yet to be tested in preclinical and then clinical studies if deemed safe. But a temporary sore throat could be preferable to lung synctitia, cytokine storm, and death.

### 6.1.3. Kinetic Transport of Molecular Niclosamide from Solution Through the Mucin

With a diameter of mucin fibers of 3 nm-10 nm [122] and mesh spacing of 20 nm-200 nm, small drug molecules like niclosamide in aqueous solution can easily diffuse essentially unhindered through mucus [122]. Applying the aqueous solution as a spray would also likely rehydrate the viscous gel on the upper part rendering it all a sol layer. An estimate of the transport time of the drug molecule through the gel can be obtained from the well-known relationship for the mean square displacement of the molecule for one dimensional diffusion, x²=2Dt. Thus, times for transport through the mucin gel scale as x²/2D, where x=the thickness of the hydrated mucin layer, which is a 15 μm thick gel [123], and D is the diffusion coefficient of a small molecule in aqueous buffer solution. As described in a very comprehensive paper by Olmsted et al, [122], various particles were tracked (macromolecules like albumin, micelles, small nanoparticles and small viruses), in rat cervical mucus, which serves here, to a first approximation, as a surrogate for nasal mucus. In some cases, the diffusion through mucus, as expected, can be slower than through just buffer, and so a factor is applied of D_{MUC}/D_{BUF}. Applying the x²=2Dt equation as 2D=x (x/t) therefore indicates the speed (x/t) with which a molecule, particle, or probe diffuses over a certain distance x, in mucus compared with saline buffer. As used by Olmsted et al [122], if this ratio 1, then the probe diffuses as rapidly in mucus as in buffer, whereas a value <1 implies that the probe is slowed by mucus.

Thus, given the diffusion time for small molecules in aqueous media (D_{BUF}˜5×10⁴ cm²/s) we would expect the molecular niclosamide to pass through as in water or buffer within 225 milli seconds. If the drug is bound to proteins like albumin (66 kDa) (which could represent a second formulation of niclosamide)(where D=0.6 ×10⁻⁶ cm²/s) Olmsted's data [122] show that most proteins (15-650 KDa) diffused as fast in mucus as they do in water (D_{muc}/D_{BUF}=0.84-1.1) and so, because of their size, are slightly slower than a small molecule drug with a diffusion time t=2 seconds. A polymer micelle of Poloxamer 407, that can somewhat interact with the polymer gel such that its D_{muc}/D_{BUF}=˜0.7, has a diffusion coefficient of 0.26 ×10⁻⁶ cm²/s, and would take 6 seconds. Any microparticle greater than the mucin cut off mesh size of ˜200 nm, (e.g., any drug microparticle of 500 nm-10 uM) would simply be limited to the mucin surface and would not permeate the mucin. And so, the fastest transport of niclosamide through the mucin gel is as a molecular solution and is the reason this niclosamide buffered solution formulation is the most optimized formulation from a drug delivery standpoint.

### 6.1.4. Timeline for the Viral Life-Cycle and SARS-CoV-2 Induced Rapid Loss of the Ciliary Layer

Highlights the Urgent Need for a Throat Spray in Addition to a Nasal Spray While, from many papers in the literature, all the components of viral and immune systems are now fairly-well characterized and understood, at least qualitatively, there seems to be a lack of attention paid to the actual kinetics involved. It is when we look to compare this viral life-cycle sequence with the kinetics of the epithelial mucosa including the action of viral replication on cell shedding that we see there is an urgent need to not only provide a prophylactic nasal spray but also a perhaps higher concentration throat spray to stop viral replication as the cells move down the back of the throat, and even kill those cells by niclosamide's apoptotic action.

While the columnar epithelia (the ciliated surface the virus infects) is not normally shed at the same rate as the squamous cell epithelia (every 2.5 hrs) it appears that viral infection can in fact induce shedding of the infected ciliated cells. Respiratory syncytial virus (RSV) promotes shedding of infected epithelial cells, and distal airway obstruction causing bronchiolitis in young children who nearly all experience at least one RSV infection by the age of 2 years [124]. This shedding of infected epithelial cells does result in reduction of measured nasal virus titers by clearing virus-infected cells from airway mucosa, but this leads to rapid accumulation of detached, pleomorphic epithelial cells, leading to acute distal airway obstruction. If virus is still active in these cells, then the disease continues in the lower airways.

Similarly, in COVID19, new paper by Robinot et al [125] entitled, “SARS-CoV-2 infection induces the dedifferentiation of multiciliated cells and impairs mucociliary clearance” shows that SARS-CoV-2 replication leads to a rapid loss of the ciliary layer. Thus, motile cilia function is compromised by SARS-CoV-2 infection, and the study identified cilia damage as a pathogenic mechanism that could facilitate SARS-CoV-2 spread to the deeper lung parenchyma.

While there appears to be some evidence that SARS-CoV-2 exerts a transient cytopathic effect on epithelial cells [125] a further hypothesis is that the shed human airway epithelial (HAE) cells are not immediately necrotic but remain functional as a mucin-trapped cell culture in which viral particles can complete their 36-hour replication cycle and emerge from the host cell as they travel in muccal secretions down into the lungs. Other data from Zhu et al [61], give even longer times for HAE cells in in vitro culture with peak virus production from apical wash at 48-72 h post infection that remained at a high level from 3 to 6 days. Data, also from Zhu et al, [61], shows that “many apoptotic cells were observed in the SARS-Cov-2-infected culture, but almost no necrotic cells were detected, indicating that SARS-Cov-2 mostly induced HAE cell apoptosis but not necrosis”. (NOTE: this is in contrast to SARS-Cov where infection was highly cytolytic; infected ciliated cells were necrotic and shed over time onto the luminal surface of the epithelium [126]).

Thus, once infected, and once shed, there is perhaps a time lag between shedding and gene regulated Apoptosis, that gives the virus time to replicate in a still functioning cell. The question now is, how long does it take to initiate apoptosis? because, as observed by Elmore, [127] the time from initiation of apoptosis to completion can occur as quickly as 2-3 hours, If the answer is 3648 hrs for initiation, then here is the mechanistic opportunity for viral replication in shed host epithelial cells and an urgent need to stop viral replication in the upper respiratory (back of the throat) as well as perhaps kill these harboring cells.

Shown in Table 2 is a tabulated timeline of events, as so far understood and reported in the literature quoted above. The table attempts to compare the kinetics of mucosal epithelium, virus entry and replication, assembly and secretion, and the delivery of Niclosamide. It exposes the urgent need and generates the hypotheses to be tested.

Starting with the kinetics of the mucosal epithelium, the 15 um thick mucin [123] is moved at 1-10 mm/hr [100] by ciliated motion, and replaced every 15 minutes [100]; epithelial cells are shed early in the infection process within 2 days of infection [125]. At the cellular (micro level) the normal rate of mucin transport is 17 um/min to 170 um/min, so ˜2 to 20 cell diameters per minute but how much is this slowed due to disruption of ciliated cells and cilia motion?

TABLE 2
Tabulated timeline of events comparing the: kinetics of mucosal epithelium, virus
entry and replication, assembly and secretion, and the delivery of Niclosamide.

| System | 3 hr | 6 hr | 9 hr | 12 hr | 15 hr | 18 hr | 21 hr | 24 hr | 27 hr | 30 hr | 33 hr | 36 hr |
|---|---|---|---|---|---|---|---|---|---|---|---|---|
| Normal Mucosal | Normal | Infected ciliated cells shed? | | | | | | | | | | |
| Epithelium | Mucin | | | | | | | | | | | |
| Mucin is moved at | replaced in | | | | | | | | | | | |
| 1-10 mm/hr, | 15 minutes; | | | | | | | | | | | |
| replaced every 15 | | | | | | | | | | | | |
| minutes | | | | | | | | | | | | |
| Infected epithelial | | | | | | | | | | | | |
| cell shedding? | | | | | | | | | | | | |
| Virus Infection: | Viral | Transcription translation using cell's | | | | | | Virus RNA | Viral assembly in Golgi | | | Virus |
| Cross mucus | diffusion | machinery and ATP takes ~24 hrs | | | | | | replicated | packaged in secretory granule | | | secreted |
| 20-30 s | through | | | | | | | | | | | |
| Cell Entry (8-12 | mucin | | | | | | | | | | | |
| mins) Replication | 20-30 s | | | | | | | | | | | |
| (24 hrs) | and Viral | | | | | | | | | | | |
| Assembly and | RNA entry | | | | | | | | | | | |
| Secretion (12 hrs) | through | | | | | | | | | | | |
| and maybe up to | endosome | | | | | | | | | | | |
| 48-72 hrs | 8-12 mins | | | | | | | | | | | |
| Niclosamide | Nic is | Raises | Dissipates pH gradient in inner | | | | | Virus not | Interferes with viral | | | Non |
| delivery (Nic): | through | pH in | mitochondrial membrane, inhibits ATP | | | | | replicated | assembly in Golgi | | | competent |
| Niclosamide | mucin in | endosome | synthesis; inhibits transcription- | | | | | | | | | virions |
| diffuses through | 275 ms | prevents | translation of viral RNA | | | | | | | | | secreted |
| mucin in 275 ms | | viral RNA | | | | | | | | | | |
| Taken up into all | | entry into | | | | | | | | | | |
| cell membranes | | cytoplasm | | | | | | | | | | |
| including Plasma, | | | | | | | | | | | | |
| endosome, | | | | | | | | | | | | |
| Mitochondria, and | | | | | | | | | | | | |
| Golgi | | | | | | | | | | | | |

As for the kinetics of viral infection, within the first 3 hrs after the virus enters the nose and contacts the mucin surface, the mucin is replaced every 15 minutes. Since the virus is calculated to diffuse through the mucin in 20-30 s, it has ample time to make this journey across the 15 um thick mucus before and as it is being replaced and moved (backwards). This assumes that the mucin is a fully hydrated sol, and so some retardation could occur for the virus to enter and cross the less hydrated and more viscous surface layers; but it clearly makes it. This is followed by kinetics of virus entry via the endosome and its fusion with the endosome membrane, releasing the RNA into the cytoplasm, (which may take ˜8-12 mins), the eclipse period of replication (˜24 hrs), and the assembly in the Golgi and secretion (another ˜12 hrs). Hence the whole process is at a minimum on the order of 36 hours total but maybe up to 48-72 hrs.

Once the virus infects this first layer of cells, it necessitates using a live cell's machinery and ATP to replicate. Any shed infected epithelia that go down the back of the throat at night could carry their replicating virus through 36 hrs after initial cell infection.

By comparison the kinetics of the niclosamide delivery is very rapid. When sprayed as a solution onto the nasal or oral mucosa, niclosamide (is calculated to) diffuse through the 15 uM thick mucin in 225 ms and is rapidly taken up because of its moderate Log P of 3.5-4, into all cell membranes including plasma, endosome, mitochondria, and Golgi. Recent cell culture experiments that add niclosamide solution in just buffer show that niclosamide starts to have its effect on ATP reduction within minutes of exposure to the soluble niclosamide [128]. Prophylactic niclosamide therefore rapidly enters the plasma membrane from which the endosomes derive. It so raises the pH in the endosome (or prevents it from being lowered) and thereby prevents spike protein rearrangement, and hence stops viral RNA entry into cytoplasm. In the mitochondrial membrane, it dissipates the H gradient across the inner mitochondrial membrane, and so inhibits ATP synthesis which in turn inhibits transcription-translation of viral RNA. The virus not replicated. If viral replication does occur and niclosamide is subsequently present, then it can enter the lipid membranes of the Golgi and interferes with viral assembly in the Golgi, such that if, and when, virions are secreted, they are non-competent and hypothetically could act as their own vaccine.

At a 6 hr exposure of human airway epithelial cells to 300 uM niclosamide, there was 83% cell death. Thus, for the throat spray we might expect some cell death during the exposure and residence of niclosamide in these cells as it also inhibits viral replication. However, this is a regenerating tissue and evidence shows that for example after (COPD) injury, the barrier function will re-establish after several days albeit still somewhat leaky[121]. Still, as mentioned earlier, is it worth risking a sore throat to prevent viral spread to the lungs? This will be determined in preclinical animal and clinical human studies.

### 6.2. Bringing this all Together: The Physiology of Viral Infection, What Niclosamide Sprays could do

The above is now all brought together in a final consideration of effective drug delivery systems for the naso and oropharynx. The development of clinically effective drug formulations must integrate the nature of the cell and tissue target, the kinetics of the viral infection, and the transport of the drug in all its potential forms. Remember, “drug solubility is everything when it comes to epithelial bioavailability”. This is especially important for low solubility drugs like niclosamide, where the aqueous solubility (of the protonated Nic_{OH}) at natural physiological pH (in this case in the nasopharynx-6.4 [108] and the saliva ˜6.7 [129] is only ˜2 uM and its efficacy against viral replication in respiratory cells is on the same order of 2 uM-3 uM [34].

First, consider the target tissue. As described by Galo et al, [130] “The nasal mucosa routinely filters, moistens, and warms the inhaled air to minimize the irritative effects on lower airways, to maintain the mucociliary clearance, and to favor gaseous exchanges”. For a given drug formulation, the biology of the tissue will ultimately determine if and to what extent any drug or drug delivery system is successful (or not) in delivering the drug to its cellular target. Nasal mucin is made up of high-molecular-weight glycoproteins that hold water. Its function is to protect the underlying nasal epithelial cells by trapping airborne particles for removal from the body through muco-ciliary clearance. It is therefore also an effective barrier for any micro-particulate drug delivery [131, 132].

The nasal respiratory epithelium contains pseudostratified columnar epithelial cells, goblet cells, basal cells, mucous, and serous glands (for review see [133]). Many of the epithelial cells are covered on their apical surface with fine projections of microvilli, which enhance the respiratory surface area. Goblet cells secrete mucins as an integral part of their function; the ciliated cells move the secreted mucin for mucociliary clearance (that actually moves the mucin backwards towards the throat and eventually the stomach); and the basal cells are the progenitors of the airway epithelium in that they can differentiate to replenish all of the epithelial cells, and so replace the epithelial mucosa on a regular basis. The timing of this replacement and the fact that it is replaced are important to both viral infection cycle and the potential use of niclosamide solution to eliminate the shed cells and also the first infected layer, that will fully regenerate anyway in a few days

Spatially and temporally, the mucin layer is 10-15 um thick [123] and the mucociliary transport moves the mucus forwards at 1-2 mm/h in the front (anterior) and 8-10 mm/h in the back (posterior) of the nasal pharynx [100]. About 20-40 mis of mucus are secreted from the normal ‘resting’ nose each day from about 160 cm of nasal mucosa. The mucin layer is replaced every 15-20 minutes due to mucin secretion and ciliated motion [100]. The top layer of squamous epithelial cells are shed every 2.5 hrs (measured from oral mucosa) [134] replacing this epithelium (300-500 μm thick, 400 cell layers) every 4-5 days. The ciliated epithelium is replaced less often, but as we saw above could be shed within the first day to 2 days of infection [125].

It is here then that the kinetics of the mucin replacement and also viral induced ciliated cell-shedding are expected to have a significant influence on the optimal design, and therefore success, of a given drug delivery system, as well as what happens to the virus after it has passed through the mucin and infected a ciliated cell in the top layer of the epithelium. Next, estimates are given for the viral life cycle and where niclosamide acts in the stages of infection.

### 6.2.1. The Macroscopic Physiological Level and Viral Spread to Organs and Tissues

Beyond single cells as described above (FIGS. 29 and 30), at the more macroscopic physiological level, to reiterate, after initial viral infection and as time goes on, the viral load in the secretions goes down and viral load in lower respiratory tract secretions rises. Again, it seems that oropharyngeal secretions are aspirated into the lungs at night during sleep, leading to the devastating COVID-19 pneumonia [135]. Once in the lungs and then the blood stream, the virus has access to all organs and tissues. Coronavirus destroys the lungs, but also damages the kidneys, heart and other organs [136]. Viral antigen (spike protein) has been detected by Liu et al, [137] in multiple organs and tissues by immunohistochemical and immunofluorescence staining. Startlingly, these include: pneumocytes and hyperplastic cells around the bronchioles; mucosal epithelia, submucosal glands, and gland ducts of the trachea; mucosal epithelia and glands of the small intestine; distal tubules and collecting ducts of the kidneys; islets of Langerhans, glands, and intra-islet ducts of the pancreas; and vascular tissues of the brain and heart. In contrast, few viral antigens were present in the large intestine and renal proximal tubules and none in the liver. As Liu reports, “Collectively, these data demonstrate direct multiorgan invasion by, or exposure to, SARS-Cov-2”.

Thus, a growing body of evidence suggests the virus invades other organs and tissues, causing heart arrhythmias, blood clots, and renal failure, as well as the brain, by crossing the BBB [138]. It also directly crosses the neural-mucosal interface in olfactory mucosa [139], creating cognitive effects brain fog and fatigue. It is here that a second formulation could have an impact, that of a Niclosamide Stearate Prodrug Therapeutic that we have developed and tested in mice [13] and dogs [59] for cancer (Osteosarcoma metastasis to lungs) but could similarly be injected intravenously for systemic exposure and tested in preclinical and then clinical studies.

### 6.2.2. Summarizing What Niclosamide Spray do could Here

It is here that the wide-spread use of nasal and throat sprays, especially prophylactically, and also in early stages of the disease, could effectively limit viral replication, viral spread to the lungs and be vital in reducing the initial viral load that the immune system to deal with (locally and systemically) and thereby help to limit, or even prevent, systemic spread, including viral transfer to the brain.

As delineated above (FIGS. 29 and 30 and Table 2) in the presence of prophylactically delivered niclosamide as a ˜pH 8 solution, niclosamide is through the mucin in 225 ms, and can partition into the cell and organelle membranes. Its presence in the endosome membrane shunts protons and raises the pH in the endosome, inhibits the low pH-dependent spike protein conformational change required for viral membrane-endosome membrane fusion and prevents viral RNA entry into cytoplasm. It also enters the mitochondrial membranes and dissipates the pH gradient across the inner mitochondrial membrane of ˜1 pH unit, inhibits ATP synthesis, and therefore inhibits transcription-translation of viral RNA. As a result, the virus is not replicated. If replication has already started or is completed, niclosamide can interfere with the assembly of the viral particles in the Golgi by dissipating its pH gradient, such that the cell eventually secretes non-competent virions.

### 6.3 A Cautionary Tale for any Micronized Material that Seeks to Deliver Drugs to Cells Underlying a Mucosa

As outlined in this patent application, the amount of a drug being delivered in the aqueous media of the target tissue is the most important goal of drug delivery, and it must be much larger than the concentrations for efficacy in the target cells. This seems obvious, but many drug delivery researchers seem to miss this in their eagerness to use their particular polymer, surfactant, liposome, dendrimer, or micronized material. Hence the need, as described here, for a comprehensive preformulation drug characterization before embarking on a “new formulation” that actually may not need the “polymer, surfactant, liposome, dendrimer, or micronized material”.

The cautionary tale is two-fold: 1) microparticles of spray dried protein with bound micronized niclosamide cannot get through the mucin network; and 2) at neutral pH niclosamide has a solubility (˜1-2 uM) that is on the same order (or less than) as the IC₁₀₀ (2 uM-3 uM) for inhibiting viral replication [34]. Here, that solubility is also influenced by the nature of the niclosamide being used and how quickly any micronized material converts and equilibrates to an even less soluble hydrate. This is clearly shown by comparing the two suppliers of niclosamide, —the AK Sci higher solubility polymorph compared to the Sigma mixed polymorph that already contains the low solubility monohydrate polymorph and so converts after dissolution when left in contact with excess material.

In our case, of using a simple niclosamide solution, any microparticles of niclosamide are eliminated and the solubility of niclosamide in the aqueous media sprayed prophylactically on the nasal mucus (20 uM at pH 8) is ten times larger than the concentrations for efficacy (2 uM) in the underlying epithelial cells, and, for the 300 uM throat spray at pH 9.2, can be up to 150 times larger.

With this in mind, what follows is a brief review of the encouraging data from Brunaugh et al [91], and a discussion of certain aspects based on the data presented in this current patent application and especially the evaluation of the nature of the mucosa and its barrier properties to microparticles. This is not to say that the Brunaugh and now Union Therapeutic's micronized niclosamide on spray dried lysozome formulation is not extremely encouraging and exciting data, that has gone on to be tested in clinical trials, but just that the performance of the microparticle formulation could be more optimized if a higher concentration of more bioavailable niclosamide in buffered solution was included. What follows now is a brief review of the microparticle Brunaugh-Union technology to distinguish it from the current patent application that is focused on a simple niclosamide solution.

### 6.3.1. NIC-hLYS Powder Shows Encouraging Efficacy in Mice

As reported by Brunaugh et al [91], the anti-MERS-CoV activity of their micronized niclosamide in spray dried lysozome in histidine buffer (NIC-hLYS) was evaluated in vivo using HDPP-4 transgenic mice. Encouragingly it did show some efficacy. Following a dose-finding study, the NIC-hLYS powder was reconstituted in 0.45% sodium chloride to achieve a dose of 240 μg/kg NIC (n=13) or. The suspensions were administered intranasally in 50 uL volumes and compared 0.9% sodium chloride as a placebo (n=8). According to their published account, “treatment was performed until 10 days p.i. (post infection), at which point surviving animals were left untreated for 3 days, and then sacrificed on day 14 p.i. to obtain tissues for viral titers and pathology”.

Results in a lethal infection model (hACE2 transgenic mice infected intranasally with a lethal dose of SARS-CoV-2 (1×10⁴ pfu) showed indeed that for a dose of 240 μg/kg NIC by day 10 (study endpoint), 30% of treated mice had survived, compared to 0% in the untreated arm. That is 70% had died. However, the results did show that the surviving mice exhibited a statistically significant decrease in viral loads in lung tissue compared to day 6 p.i. untreated controls and lower cases of pneumonia than for infected and non-treated mice. Also, no virus particles were detected in brain and kidney tissue using qPCR.

Again, this is very encouraging, for the current niclosamide solution, because it shows that niclosamide can have a positive effect in vivo. The question now though is, could the surviving fraction be raised if the drug was delivered at a higher niclosamide solution concentration, (not necessarily a higher μg/kg)?

Note: for the pH 8 solution prophylactic at our anticipated epithelial-safe concentrations of 20 uM-30 uM, if given to mice in the same 50 uL volumes (which is more than the usually acceptable intranasal dose of 24 uL-2×6 uL per nostril), the dosing would be 0.327 ug. For a 20 gm mouse (0.02 kg) that would be 16.4 ug/kg compared to the 240 ug/kg niclosamide delivered in the NIC-hLYS. Note, their niclosamide per lysozyme is only present at 0.7% by weight. The problem gets worse though for the NIC-hLYS formulation, because out of this 240 μg/kg niclosamide only the soluble fraction is bioavailable, in the apparent aqueous 0.45% NaCl suspension it is delivered in.

And so, the data and invention outlined in this current patent application would be the optimization of delivering a solution at a higher soluble fraction of niclosamide than a micronized-lysozyme spray dried suspension that would be likely to get stuck at the mucosal surfaces with minimal niclosamide in solution that could permeate the mucin. This is now all discussed in more detail below.

### 6.3.2. Microparticles do not Get Through the Mucin Network and any Niclosamide in Solution is at or Below Efficacious Levels

In the Brunaugh-TFF-Union formulation [91, 140], in order to generate a powder formulation of niclosamide, micronized niclosamide particles were embedded in a matrix of recombinant human lysozyme, that also included sucrose, polysorbate 80 and Histidine (buffer) using spray drying. The concentration of niclosamide in the NIC-hLYS powder was actually only 0.7%, meaning the spray dried particles for a 60 mg total powder actuation were 99.3% lysozyme, 0.7% niclosamide and other excipients, i.e., 420 ug of niclosamide particles per actuation. While initially developed as a dry powder for inhalation, it seems the dry particles were then reconstituted as an aqueous suspension for nasal spray or nebulizer-based administration. Although not discussed by Brunaugh et al, upon resuspension in aqueous buffer some of the niclosamide can clearly dissolve up to its solubility limit in the Histidine buffer at a pH of 6.0. From the data presented in this current patent application (FIG. 11B and the pHp model) this is expected to be a quite low concentration of only 1 uM at pH 6, if the supplied niclosamide already was or had converted to its monohydrate form. The Brunaugh niclosamide was purchased from a company in China, Shenzhen Neconn Pharmtechs Ltd. (Shenzhen, China), that is also of undefined crystal morphology, and hence solubility, and has not been measured as a function of pH. In any event, the amount of niclosamide that is bioavailable to the actual epithelial cells in this resuspended micronized spray dried-lysozyme formulation is only on the same order as, or perhaps less than the measured efficacy of niclosamide in Calu-3 cells of 2 uM-3 uM [34].

In order to exemplify and delineate the issues, shown in the schematic in FIG. 31, is the mucin layer covering the first layer of nasal epithelial cells. The two schematics compare a micronized niclosamide delivery and a solution of niclosamide with respect to mucin penetration. The mucin layer is assumed to be similar in thickness to that reported be Beule [123] in humans (10 uM-15 uM).

FIG. 31A shows the expected situation for administration of a suspension of spray dried lysozyme protein particles with a small amount of micronized niclosamide (at only 0.7%) apparently attached that, from the images of the Brunaugh paper itself [91], although quoted as (diameter ≤5 um), are clearly 5-10 um and some are lager.

Even so, as such, the spray dried particles would not permeate the mucin gel network that has a size cut off of at most, 500 nm. Any drug delivery of niclosamide therefore must rely on the micronized niclosamide dissolving in the suspension solution to its solubility limit (of 1-2 uM) and permeating the gel as dissolved molecular species. Hence the mucin gel would likely contain some niclosamide and so is shaded lightly grey. With a Log P of 3.5 at this nasal pH, we might also expect some partitioning into the lipid bilayers of the mucosa (light grey outlines), and given time to reach equilibrium, this may be enough to show some efficacy. However, this is clearly not optimal compared to a simple niclosamide solution that readily permeates the mucin and delivers 20 uM niclosamide directly to the epithelial cells as shown in FIG. 31B.

Here, microdroplets of niclosamide from the spray that can be in the range 20 um to 30 um in diameter and up to 100 um in diameter, (shown in solid grey) would spread on the outer surface of the mucin at its air-mucin interface. Since this interface is a less hydrated and more viscous gel, the niclosamide solution would help to rehydrate the mucin and provide a well hydrated sol for niclosamide molecular diffusive transport to the cells in a calculated 225 ms. Thus, the mucin would now contain much more niclosamide (hence the more intense grey shading in the schematic) and provide for more rapid partitioning into the cell membranes and their organelles including endosomes, mitochondria, and Golgi (not shown).

### 6.3.3. In Aqueous Suspension, Lysozyme could Act as a Molecular Carrier, but its Positive Charge would Bind it to the Mucin not Necessarily Permeate it

Another aspect of the Brunaugh studies deserves some analysis and brief comment because this also impacts the delivery in vivo. While the in the in vitro cell studies it was considered a plus that niclosamide-embedded in the spray dried lysozyme formulation showed some activity of lysozyme itself, this was in the absence of any mucin layer, i.e., it was done on Vero 6 cells in submerged culture, as opposed to Air/Liquid interface epithelial cells that can develop a mucin layer. Upon resuspension in low pH 5.0-5.2 histidine aqueous buffer, one could speculate that some of the lysozyme could dissolve, and given that niclosamide does bind to proteins, it could bind niclosamide, and this could be enhanced by an ionic interaction between the negatively charged niclosamide and the positively charged lysozyme. This could therefore be a good thing since the lysozyme could act as a molecular carrier that could in principle permeate the mucin. However, the mucin is negatively charged and so the positively lysozyme (and its bound niclosamide) would likely adhere to mucin and not permeate to the epithelial cells. Thus, while lysozyme may have a beneficial effect on cultured Vero 6 cells that do not express mucin, in the in vivo situation, even the lysozyme would not be expected to reach the epithelia.

### 6.3.4. When in Equilibrium with Particulate Niclosamide the Amount of Niclosamide in Solution is Expected to be Less than the IC₁₀₀ to Inhibit Viral Replication

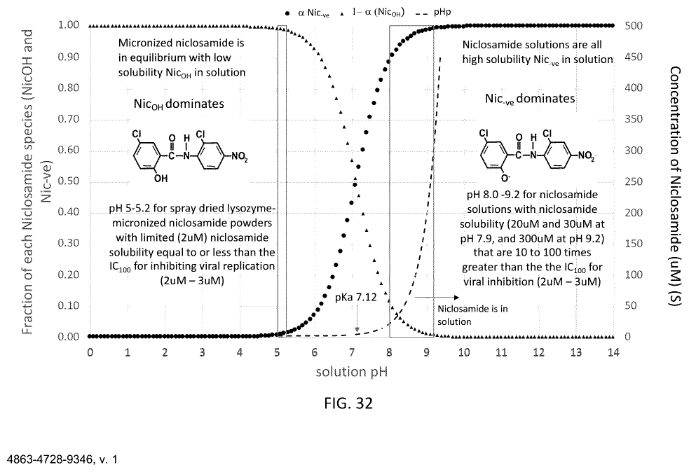

As shown in FIG. 32, is a comparison of the pH-ranges of the solutions for the two formulations.

FIG. 32 is created by taking FIG. 4 for the composite HH and pHp curves giving the corresponding amount of niclosamide in solution and modifying it to show the pH ranges and hence niclosamide in solution for the Brunaugh spray dried lysozyme-micronized niclosamide powder [91] and the niclosamide solutions made here at pH 8.0 for prophylaxis and pH 9.2 for throat early treatment. The predominant species that underly these amounts of niclosamide in solution are shown in the grey blocks.

The Brunaugh spray dried lysozyme with 0.7% micronized niclosamide attached [91] is made at ˜pH 5.0-5.2 with histidine as the buffer [141, 142]. Assuming the histidine is still in the powder when the aqueous suspension is made for the nasal spray and inhalant, the amount of niclosamide expected to be in solution at this pH 5 to 5.2 is ˜1 uM-2 uM depending on the polymorphic source. Thus, at this pH the micronized protein-spray dried niclosamide is in equilibrium with low solubility (1 uM) Nic_{OH} in solution. And so, this compromises the bioavailability of molecular niclosamide to nasal epithelial cells, since it is on the same order as or less, than, the IC₁₀₀ (2 μM to 3 μM) for inhibiting viral replication in Calu-3 cells [34]. In contrast, the niclosamide solutions made here provide an amount of niclosamide in solution of 20 uM to 30 uM at pH 8.0, and 300 μM at pH 9.2, and so are 10 to 100 times greater than the IC₁₀₀ for viral inhibition (2 μM-3 μM) [34]. Also, when made by the solvent exchange technique they are not exposed to or affected by any crystalline material that can reduce these solubilities, at least in the near term.

### 6.3.5. Vero 6 Cells are not the Best Choice for Testing Drugs Against Viral Infection

Finally, as suggested by Murgolo et al, [143], Vero 6 cells are deficient in expression of angiotensin converting enzyme 2 (ACE2) and TMPRSS2, and the virus probably uses other nonspecific endocytic uptake mechanisms for viral entry. However, more recent data [144] shows that ACE2 expression was observed in Vero-E6, Calu-3, and Caco2 that correlated with high virus production. Additionally, co-expression of ACE2 and TMPRSS2 was most prominent in Caco2, followed by Vero-E6 and Calu-3, although Calu-3 showed low TMPRSS2 expression. Thus, the antiviral activity seen by the micronized niclosamide, especially associated with any blocking of endosome lysozome-autophagy pathways seen in Vero 6 cells, may or may not translate to primary lung epithelial cells. In any event, from Ko [34], the IC₅₀ for niclosamide in limiting viral replication was 3 times higher in Calu-3 lung cells (0.84 uM) than in Vero 6 cells (0.28 uM) as measured by Jeon et al. The IC₁₀₀ was also higher at ˜2-3 uM. And so, this is higher than the equilibrium amount of niclosamide expected to be in solution (˜1-2 uM) at pH 5 for the micronized material.

### 6.3. The Ultimate Goal is to Motivate Preclinical and Clinical Testing

What this analysis leads to is the conclusion that a niclosamide solution (as opposed to any particulate material) at the higher buffered pHs of only 8.0 and 9.2, could provide a much more optimized and mucin-penetrating solution of niclosamide for prophylactic nasal and early throat treatment options.

This now warrants efforts to evaluate the simple solutions, in more appropriate epithelial cells, preclinical animals, and human clinical trials The ultimate goal is to motivate preclinical and clinical testing of the simple niclosamide solution in order to move the solution into human trials and for the niclosamide solution to become available world-wide, preferably at cost or reinvested profit. As discussed, because of niclosamide's proton shunt mechanism, Niclosamide is a virostatic as opposed to an anti-viral per se, i.e., it targets the host cell rather than the virus itself. This means that the availability of such a simple solution and optimized formulation would be especially important, for example: in regions without enough or any current vaccines against this current coronavirus; it's emerging and more contagious variants such as the Delta variant [7]; and to prepare for the next one, including seasonal influenza as well as other respiratory viral infections.

The envisioned final product is a solution of Niclosamide made up in pH buffer and available in 10 mL doses in 15 mL nasal and throat spray bottles. As mentioned above our preliminary data in airway epithelial cells [45] suggest that a 20 uM niclosamide solution at pH 8.0, which is within the upper end of nasal pH of 5.17-8.13 [108], could successfully reduce the ATP per cell while still maintaining reversible viability. As a mucin-penetrating solution it can achieve direct access to nasal and upper respiratory epithelial cells that are coated with a mucin barrier. While micellization with surfactants, binding to albumin, or stabilized nano- and micro-particles can also do this [16] the most effective and least complicated mucin-penetrating formulation, is a niclosamide solution. where it is stable in an aqueous solution for several months [145]

As demonstrated in this paper, this was achieved by measuring niclosamide solubility vs pH for a commercially available niclosamide powder (from AK Sci, CA) and also readily extractable from already approved and commercially available niclosamide tablets, such as Bayer's Yomesan and other generics. For a pKa determined here of 7.12, according to the Henderson Hasselbalch equation, at pH 8.0 we can achieve a concentration of 20 uM, and, at pH 9.2, where the Niclosamide salt dominates, the effective amount of niclosamide in solution is raised to ˜300 uM. Thus, the product is a simple Niclosamide solution that can be sprayed intranasally and to the back of the throat from common spray bottles. At pH 9.2 for the oral spray, it's pH is equivalent to that of green tea and bottled alkaline water and so the pH levels should be safe. Clinically, the solution could be drawn from capped and sealed sterile 10 mL vials into a syringe and administered intranasally and to the throat from, for example, Teleflex Nasal and Laryngeal atomizers [146]. Eventually as an OTC medication for world-wide distribution, it could be filled and finished into nasal and throat spray containers with no cold chain, as used in other commercially available products like hydrating nasal solutions, Flonase, and Nasonex. A list of potential clinical trial populations and end users who might benefit from the prophylaxis and early treatment nasal and throat sprays is given in section 7.2.4.

### 7. Suggestions for Future Work: Hypotheses to be Tested in Cells, Preclinical, GLP Tox and on to Clinical Testing

What should be obvious after reading through the above motivation, literature reviews, experimental data, results, and discussion is that there is still lots of work to do to make the title a reality, “a New Nasal and Throat Spray Solution-Formula don for COVID19 and Other Viral Infections”. There are clearly new mechanisms to be tested for niclosamide in especially airway epithelial cells (as opposed to just Vero 6 and Calu-3 cells), and efficacy and safety to be demonstrated and confirmed in animals and in human trials.

### 7.1. Hypotheses to be Tested

This section lays out just some of the hypotheses that researchers could take on and propose to test now, if bootstrap funding is available, or in new grant proposals to fund the work. The main hypothesis that motivated the current study and its subsequent testing is “a simple niclosamide solution, when used as prophylactic nasal and early treatment throat sprays could reduce the viral load that the vaccinated and unvaccinated immune system has to deal with” As reviewed, many of the in vitro studies that evaluated viral infection in cells and the effects of drugs including niclosamide were largely done on the robust Vero 6 cells (monkey kidney cells).

Even the lung derived (from cancer) Calu-3 cells are still not that appropriate. And so, it is clear that, in order to learn more about the role of niclosamide in its multiple inhibitory actions both in the healthy host cell per se and in a virally infected or infectable cell, airway epithelial cells that the virus actually infects should be the next test systems. And these should not be just airway cells in submerged culture, but cells grown in special cultures at the Air Liquid Interface (ALI), that generate and maintain a mucin layer and so can be used to test drug delivery of solutions, nano and microparticles to determine which one or more is the best. Here we are getting into the realm of research grant proposals, that we have already started to propose and test, and the author is quite happy to share as much as possible in order to stimulate others to join the process, write, submit, and successfully fund their own studies. For example, a recent submission [147] was to characterize five prototypical formulations of niclosamide [3, 148-156], and test them for ATP-reduction, drug-deposition, SARS-CoV-2 activity and, importantly, therapeutic index in airway epithelial cells.

Thus, the work here has generated new hypotheses to be tested, especially in airway epithelial cells that the virus infects. In no particular order, these include: 1) the effect of niclosamide in the local immune system (good or bad?) 2) could the virus (membrane) loaded with niclosamide even be its own drug delivery system? 3) are infected cells self-selective for niclosamide because they contain more lipid droplets? 4) what is the effect of niclosamide on mucin production and movement, —could it slow it and trap the virus? 5) could misassembled non-competent virions act as their own vaccine? And 6) are the shed epithelial cells able to culture the virus during its replication cycle?

### 7.1.1 The Effect of Niclosamide in the Local Immune System (is it Good or Bad?)

For example, what about an over reactive immune response and the effect of niclosamide on NALT? There is the issue of the cytokine storm and an over reactive immune system, even locally in the nose and throat. Gallo et al [130] report that “At the mucosal level, SARS-Cov-2 is a cytopathic virus able to induce death and injury to infected tissues by eliciting a highly inflammatory form of programmed cell death termed pyroptosis”. Since niclosamide has an effect on all cells and reduces all ATP, even in immune cells, another hypothesis to be tested therefore is, would the presence of niclosamide also quell some of this over reaction and subsequent damage from the “inflammatory form of programmed cell death” ? Could niclosamide be titrated at levels that inhibit viral replication and also stop the increased secretion of several cytokines and chemokines (IL-6, IFN-gamma, MCP1, and IP-10) associated with hyper-inflammation, which are released into the blood. Hence what is the effect (both positive and perhaps negative) of niclosamide on the Nasopharynx-Associated Lymphoid Tissue (NALT) system, that first recognizes exogenous airborne agents, and its efficacious and inefficacious immune response?

### 7.1.2 could the Virus (Membrane) Loaded with Niclosamide Even be its Own Drug Delivery System?

It is an interesting hypothesis that the virus could actually be its own drug delivery system if it were saturated with niclosamide due to spraying the mucosa in the nose and throat. The calculation starts by recognizing that, as stated in the preformulation drug characterization section, its log P is 3.5 at neutral pH, because even though it acquires a negative charge, this charge is distributed in internal hydrogen bonds and so the molecule is a lipophilic anion that can readily enter membranes. In fact, as should be clear now, this is the whole basis for its action as a proton shunt, —a lipophilic anion that can partition into membranes and bind and move positively charged hydrogen ions down the cell's set-up pH gradients in the endosome, mitochondria, and Golgi. A log P of 3.5 means that it will partition into octanol at a ratio of 1:3,200 times more into octanol than in water. With a solubility of ˜1-2 uM at this neutral pH (as measured here), at equilibrium between octanol and a 1 uM solution there is a concentration of niclosamide in octanol of ˜3.2 mM. Niclosamide is a small molecule with a relatively low topological polar surface area. A presented earlier in Preformulation drug characterization, its molecular volume Vs of 202.5±3.0 cm³, (ACD-iLabs) is well within the parameters for membrane partitioning, and, with a low topological polar surface area of 95.15 Å² (Drugbank), where any value below 140 Å² is considered hydrophobic enough to partition into the membrane interior, it could in principle partition into membranes at this same concentration (3 mM).

A simple geometric calculation of the number of lipids per membrane volume of a 100 nm diameter lipid bilayer gives the concentration of lipids as 4.3 mM. And so theoretically niclosamide could partition into the lipid bilayer membrane of the virus at a concentration of 3.2 mM and a lipid to niclosamide mol:mol of 1:0.74.

If we now imagine the whole epithelial mucosa being saturated by 20 uM niclosamide in the nose and 300 uM niclosamide in the throat, any lipid membrane coated virus would also take up niclosamide at, at least, its partitioning associated with neutral pH of these environments but could be higher until the pH actually equilibrates with mucus and saliva. Any of these free, mucin-located, viruses that then bind and enter the next epithelial cell would bring with it, its own niclosamide, that could partition into the endosome membrane and carry out its proton shunt functions of dissipating that pH gradient.

Thus, because niclosamide readily enters lipid membranes, and the SARS-COV-2 virus is a membrane encapsulated virus, niclosamide will enter its membranes too. Hence by spraying a solution of niclosamide on the epithelial mucosa, that contains copies of the virus, the virus could be saturated by niclosamide and be itself be its own drug delivery system, carrying niclosamide into the cells it infects. This is certainly a hypothesis worth testing in future virus-cell in vitro studies to determine, by analytical methods (HPLC-LCMS), if and to what extent this partitioning occurs and the concentration of niclosamide per lipid bilayer-coated viral particle.

### 7.1.3 are Infected Cells Self-Selective for Niclosamide Because they Contain More Lipid Droplets?

Another interesting hypothesis that infected cells could be self-selective for niclosamide because of their greater hydrophobic content in the form of lipid droplets, —dedicated lipid droplet (LD) organelles for normal lipid handling [157]. While initially thought to just be specialized intracellular compartment dedicated to lipid storage, new evidence suggests that they can exert a more generalized role in cellular stress response. It is now found that proteins bind to LDs and determine their function, and direct multiple contact sites formed by LDs including proximity to mitochondria.

Thus, related to the partitioning of niclosamide into lipid membranes because of its moderately high Log P (and Log B), and its lipophilic anion characteristic, is the observation that SARS-CoV-2-infected cells both in vitro and in vivo in the lungs of COVID19 patients are seen to form intracellular lipid droplets [158]. As reported by Nardacci et al, “Type II pneumocytes in lung tissue showed prominent altered features with numerous vacuoles and swollen mitochondria with presence of abundant lipid droplets”. Coronavirus infected patients showed an alteration in blood cholesterol and lipid metabolism, and statins have been shown to suppress viral replication. The lipid droplets seen in SARS-CoV-2-infected cells appear to be associated with mitochondria, and that such “proximity of mitochondria and lipid droplets is necessary for the ATP production, via β-oxidation”. The hypothesis is then that infected cells would, presumably, take up more niclosamide than non-infected cells because of this increased amount of lipid droplet “solvent” for the hydrophobic niclosamide. If so this niclosamide would be associated with the mitochondria where it has its greatest effect on ATP production. Shutting this production down at site with niclosamide, deprives the adjacent virus the all-important energy of ATP.

### 7.1.4 could Niclosamide have a Positive Effect on Mucin Production and Movement, Trapping the Virus?

The hypothesis here is that niclosamide may slow ciliated (ATP dependent) movement of the mucin and keep everything trapped. That is, its effects on the cell are to reduce ATP per cell, and so this would not only limit transcription translation, but also all cell processes, including the production of mucin by the goblet cells and its movement by ciliated cell transport. It remains to be tested if this mechanism can have positive effects in limiting viral spread in mucus secretions.

### 7.1.5. Could Misassembled Non-Competent Virions Act as their Own Vaccine?

The observation that niclosamide also inhibits the manufacture of viral protein assembly in the pH-dependent activity of the Golgi [65], also suggests an interesting possibility to test in the SARS-Cov-2-niclosamide system. Thus, could non-competent virions hypothetically act as their own “vaccine”, i.e., if they are non-competent because of mis-assembly and yet still express viral antigens like the Spike protein or other proteins, could these stimulate an immune response similar to that mounted after more traditional vaccination. In unvaccinated people, this could be an immune boost, and even in vaccinated it could add to their immune responses during subsequent infections if niclosamide spray is used after known exposure or break through symptoms.

### 7.1.6. Are the Shed Epithelial Cells Able to Culture the Virus During its Replication Cycle?

The hypothesis here is that, shed human airway epithelial (HAE) cells are not immediately necrotic but remain functional as a mucin-trapped cell culture in which viral particles can complete their 36-hour replication cycle and emerge from the host cell as they travel in muccal secretions down into the lungs. As stated above and reiterated here, once a nasal epithelium cell in the first layer under the mucin is infected, and shed within ˜1-2 days, it must incubate the virus for at least 36 hrs if the virus is to replicate and multiple copies are to be released. And so, are these shed epithelial cells able to culture the virus during its replication cycle? Is there enough time before they go Apoptotic for the virus to be replicated and be secreted? And, if treated by the higher dose of 300 uM niclosamide solution that also drives the cells perhaps more quickly into apoptosis, could their Apoptotic state also signal an immune response to clean them up, including their contained non-replicating viral particles?

### 7.2. Animal Testing, Regulatory Issues, and Clinical Testing

### 7.2.1 Preclinical Animal Studies

In other grant proposals we are currently proposing to evaluate the efficacy of niclosamide as a prophylactic and early therapeutic in models of SARS-CoV-2 [159] and extending to early COVID19 and other influenza infection in rodents [160]. The goal here is to determine the effects of escalating doses of niclosamide in vivo on epithelial cells in the nasopharynx and oropharynx of rodents. Such studies could also be extended to other viral infections and the obvious one is influenza. While new vaccines have to be provided every year against multiple influenza virus epitopes because of the continual evolution of new variants, a simple sprayed solution of niclosamide could combat any virus or variant, because of its ubiquitous activity on as a host cell modulator. This has generated another proposal that also looks more forward to commercialization as an SBIR [161] focused on influenza as a first indication. Other researchers are encouraged to join us and submit their own proposals in their own specialized models for evaluating the efficacy and safety of niclosamide solutions as described and made here.

### 7.2.2. Niclosamide Development and Approval

One of the main issues for development and approval of niclosamide in the USA is that, while it was an approved drug made by Bayer, its regulatory status lapsed in the mid 1990s. Any new formulation for a new indication such as a nasal and throat spray for COVID19 must therefore undergo 502b regulatory approval; it's almost like starting again at the FDA, as they informed us when they responded to our preIND letter last July 2020. Outside the USA however, niclosamide is approved and used throughout the world. Given this approved and repurposed status, being used in the throat where it contacts the epithelium anyway as 2 grams of “well chewed” tablets, the challenge now is to negotiate the required pre-clinical, IND-enabling safety and efficacy studies that need to be done to satisfy the local regulatory agencies.

As mentioned earlier (Initial Thinking 3.2), for the 300 uM throat spray, (the highest dosing spray) the dose that is administered in 100 uL is just 9.8 ug per dose. This is a tiny amount of niclosamide. By comparison, as discussed above, the normal oral dose of niclosamide in the Yomesan tablets is 2 gms but it is the bioavailable fraction that the epithelia are actually exposed to. This is calculated to be 19.6 micrograms. For the nasal spray, 100 uL of a 20 uM niclosamide solution delivers only 0.65 ug to the nasal epithelium.

### 7.2.3 GLP Tax Studies in Dogs?

While the FDA in the USA views niclosamide as a “new” drug and has required GLP Tox studies in dogs, could an argument be made to forego such studies in other countries where Niclosamide is still an approved drug, is routinely prescribed for worms, is readily available, and where vaccines against viral infection and more contagious variants are lacking or non-existent? The argument to be made centers around the miniscule amounts of niclosamide that are actually used in the nasal and throat sprays, that are unlikely to amount to enough of niclosamide having any systemic effects, and effects being localized to the nose and throat.

Given the ultralow dosing of only 0.65 ug per dose (100 uL of a 20 uM niclosamide solution) in the nose and 9.8 ug per sprayed dose in the throat, for a 70 kg person this represents a dose of 9.2 ng/kg and 140 ng/kg respectively. If all of this went instantaneously systemic, for a blood volume per human male of 70 kg of 5.25 L, the instantaneous maximum concentration of niclosamide in the systemic circulation would be 120 ng/mL, i.e., 0.38 nM and 1.87 ug/mL and 5.7 nM respectively. The numbers speak for themselves. Actual PK considerations of bioavailability from nasal and respiratory mucosa and rates of absorption would significantly reduce these Cₘₐₓ estimates.

It is worth considering then, as above, what the usually required GLP-Tox studies might find, if anything. These studies are not cheap, ˜$1M for a series of dogs to be exposed to the spray doses, killed and comprehensive measures of the amount of niclosamide that could be found in their organs and tissues. And so, the above calculation is presented in case anybody taking this formulation further wants to try and make the case that GLP studies are somewhat moot.

Toxicity in the oral cavity is expected to be minimal if at all, given the comparison made above that, in humans, from the 2 grams chewed thoroughly in the mouth for worms, the bioavailable amount (19.6 micrograms) is till greater than the 9.8 ug per dose that is sprayed in the early treatment regimen.

For the nose, even though 100 uL of a 20 uM niclosamide solution delivers only 0.65 ug to the nasal epithelium, clearly local toxicity is warranted, especially since the nasal bulb is a direct route to the brain. If niclosamide is found here, and as long as there are no deleterious inflammatory reactions (which niclosamide should quell anyway) this might provide some evidence that niclosamide could also reduce viral infection in the brain, which would be a good thing. In any event, all of these kinds of tox and efficacy results should be revealed in preclinical small animal studies and a considered evaluation made by the regulatory agencies in any country that wants to develop and pursue this formulation for its own purposes.

### 7.2.4. Clinical Testing and Adoption

All the above is designed to provide the preclinical data necessary to obtain Investigational New Drug (IND) approval from the FDA in the USA, and possibly more expedited approval in other countries where niclosamide still has regulatory approval as oral tablets (Bayer and generics) and with other drugs in powdered mixtures. It will allow initiation of early phase clinical trials testing safety and efficacy of niclosamide in humans. While some say, it is more difficult to prove a preventative than a cure, it was done for the multiple vaccines.

As we evaluate how to establish clinical testing, there are many populations that could be considered for testing and then could benefit from a nasal spray prophylaxis and early treatment regimen if they initially contract the disease. The CDC is mainly concerned, and rightly so, with vaccinations and the almost complete protection this has provided. However, according to OurWorldinData.org, [162] currently in the USA the number of people fully vaccinated (Nov. 7, 2021) is 192.4M, and so still only 57.2% of the total population. While Canada, the UK, Spain Italy, Germany, and France have numbers at 74.8%, 67.2%, 81.5%, 77.4%, 69.1% and 76% respectively, across the world, the average fully vaccinated is now (40.6% 3.13 billion/7.71) billion. However, only 4.2% of people in low-income countries have received at least one dose, and, for example, in Africa only 6.2% are fully vaccinated.

Within these general numbers there are categories of people that are more exposed to the SARS-COV-2 virus and others. Thus, in each category there could be a case made for additional protection and early treatment using a niclosamide spray solution to help in mitigation strategies. The categories listed below include the obvious and more detailed recommendations from the CDC, as referenced herein:
Front line workers who are likely to still be wearing a mask even if vaccinated. Here the nasal spray could act as a prophylaxis, as protection behind the mask or, depending on clinical trial results, maybe instead of it.
Older unvaccinated adults [163] who are more likely to need hospitalization, intensive care, or a ventilator to help them breathe, or they might even die
Members of the general population [164] who venture out into at risk environments and who are similarly already vaccinated. As warned by the CDC, [69] such vaccinated individuals could, if they get exposed to the virus from another person or persons, become asymptomatic carriers and spreaders, or have break through infections.
Vaccine hesitant and vaccine cautious individuals might consider a nasal spray prophylaxis to help mitigate their protection,
Travel is also starting up again and the CDC still recommends [165] that during travel wearing a mask over the nose and mouth “is required on planes, buses, trains, and other forms of public transportation traveling into. within, or out of the United States and while indoors at U.S. transportation hubs such as airports and stations”.
Children under 12. With the vaccine now being tested in children for those under 12 it is only starting to become available, and so the CDC [166] recommends that, “Children between the ages of 2 and 12 should wear a mask in public spaces and around people they don't hire with”.
As published by the CDC [167], “Adults of any age with the following conditions can be more likely to gel severely ill from COVID-19, meaning they “may need, Hospitalization, Intensive care, A ventilator to help them breathe, or they may even die”. Conditions considered most at risk include:
   Cancer (treatments for many types of cancer can weaken the body's ability to fight off disease);
   Chronic kidney disease;
   Chronic lung diseases (COPD, asthma, interstitial lung disease, cystic fibrosis, and luminary hypertension);
   Neurological conditions, such as dementia;
   Diabetes (type 1 or type 2), Down syndrome; Heart conditions, (heart failure, coronary artery disease, cardiomyopathies, and possibly high blood pressure (hypertension));
   HIV infection, Immunocompromised state (weakened immune system because of Many conditions and treatments, including inherited genetic defects or Prolonged use of corticosteroids or other immune weakening medicine);
   Liver disease (alcohol-related liver disease, nonalcoholic fatty liver disease, and especially cirrhosis);
   Overweight and obesity (elevated BMI);
   Pregnancy and recently pregnant (for at least 42 days following end of pregnancy);
   Sickle cell disease or thalassemia;
   Smoking, (current or former);
   Solid organ or blood stem cell transplant, (including bone marrow transplants)
   Stroke or cerebrovascular disease, (affects blood flow to the brain) and
   Substance use disorder (alcohol, opioid, or cocaine use disorder)

### 8. Overall Summary and Conclusions

What is offered here is a low dose prophylactic nasal spray that would stop replication of the virus at its point of entry (in the nose) and a higher concentration throat spray that would reduce viral load as it progresses down the back of the throat. It now needs testing.

The three main experimental conclusions for the formulation are that: 1) the amount of niclosamide in aqueous buffered solution can be increased beyond its low solubility at pH 7 by simply increasing the pH of the aqueous solution; 2) The rate of dissolution for a nominal 1 mM niclosamide powdered niclosamide over a pH range from 8.62 to 9.44 increased by a factor of just over 3×. A more optimized stirring gave a further increased rate of dissolution as expected from dissolution models. 3) Niclosamide can be readily extracted from commercially available Yomesan and generic tablets which could have an impact in solution preparations of already regulatory approved materials. 4) Some suppliers provide niclosamide as a more soluble polymorph (referred to here as the high solubility brick like AK Sci polymorph with an intrinsic solubility measured as 2.53 uM at pH 3.66. This compares to other suppliers (e.g., Sigma) that provide a mixed polymorph powder that contains both the brick like polymorph and the lower solubility needle-shaped monohydrate polymorph (intrinsic solubility ˜1 uM) such that when dissolved and equilibrated with excess niclosamide the undissolved material converts to the lower solubility monohydrate and lowers the amount in aqueous buffered solution. 5) The assumed monohydrate polymorph is also formed by precipitation from supersaturated solution. 6) Acetone and ethanol cosolvates also show the needle-shaped morphology and low solubility polymorphs.

Therapeutically, niclosamide can be made into nasal spray and throat spray solutions at pHs that are still acceptable for the nose and throat, i.e., for a prophylactic nasal spray 20 uM-30 uM niclosamide is obtained at pH 7.96 and 8.16 (according to the pHp theory fit to the data in FIG. 11B), and for a throat spray solution, niclosamide concentration can be increased up to 300 uM at pH 9.2. Concentrations as high as 703 uM can be obtained at pH 9.63. The reason these concentrations are chosen is based on the preliminary data from Kim et al that showed a reduction of ATP to 50% could be obtained at only 20 uM-30 uM that is 10× the IC₁₀₀ for inhibiting viral replication, and this is not lethal to airway epithelial cells that are completely recoverable after drug wash out. 300 uM niclosamide though reduced the host cell ATP to 30%, and only 17% cells were recoverable, and so 83% went probably into apoptosis. Used now in early treatment as a throat spray, the host cell's ability to replicate viruses would be completely stopped, and virus load in the lungs could be reduced. There is considerable evidence in the literature to suggest that niclosamide used in this spray context could inhibit three of the six stages of viral infection, namely, uncoating, replication and the assembly of competent virions and discussed at length in FIGS. 29 and 30.

Again, what should now be obvious after reading through the above motivation, literature reviews, experimental data, results, analyses, and discussion is that there is still lots of work to do to make the title “a New Nasal aid Throat Spray Solution-Formulation for COVID19 and Other Viral Infections” a reality. There are clearly new mechanisms to be tested for niclosamide in especially airway epithelial cells (as opposed to just Vero 6 and Calu-3 cells). The main hypothesis that motivated the current study and its subsequent testing is “a simple niclosamide solution, when used as prophylactic nasal spray could prevent initial infection and as an early treatment throat spray could reduce the viral load that the vaccinated and unvaccinated immune system has to deal with”. The work has also generated new hypotheses to be tested associated with: 1) the effect of niclosamide in the local immune system (good or bad?). 2) could the virus (membrane) loaded with niclosamide even be its own drug delivery system? 3) are infected cells self-selective for niclosamide because they contain more lipid droplets? 4) what is the effect of niclosamide on mucin production and movement, —could it slow it and trap the virus? 5) could misassembled non-competent virions act as their own vaccine?

Efficacy and safety still need to be demonstrated and confirmed in preclinical animals and in human clinical trials. Here, preclinical studies could evaluate the efficacy of niclosamide as a prophylactic and early therapeutic solutions in models of SARS-CoV-2 [159] and because niclosamide is a host cell modulator extend to other infection such as influenza [160] and other respiratory viruses and their variants. From a regulatory standpoint, because approval for Bayer's Yomesan was withdrawn by the US FDA in the mid 1990s, any new formulation for a new indication such as a nasal and throat spray for COVID19 must undergo 502b regulatory approval. Outside the USA however, niclosamide is approved and used throughout the world, and so approval could be perhaps expedited.

Finally, regarding use and influencing policy, as of November 2021, while Canada, the UK, Spain Italy, Germany, France, and the USA have vaccination numbers at 62% to 81%, across the world, the population average for fully vaccinated is only 40.6% and only 4.2% of people in low-income countries have received at least one dose. Thus, a case can be made for additional protection and early treatment using niclosamide spray solutions to help in mitigation strategies until greater vaccination can be achieved. A series of categories for clinical testing and use would follow recommendations from the CDC, including “essential” workers [168], older unvaccinated adults [163], members of the general population [164], vaccine hesitant and vaccine cautious individuals, travel [165], children under 12, and a whole host of individuals with underlying conditions [167], who “may need, Hospitalization, Intensive care, A ventilator to help them breathe, or they may even die”. The ultimate goal of this patent application is to motivate preclinical and clinical testing of the simple niclosamide solution in order to move the solution into human trials and for the niclosamide solution to become available world-wide, preferably at cost or reinvested profit [169].

### REFERENCES CITED

1. Hou, Y. J., et al., SARS-CoV-2 Reverse Genetics Reveals a Variable Infection Gradient in the Respiratory Tract. Cell, 2020. 182(2): p. 429-446.e14.
2. Docea, A. O., et al., A new threat from an old enemy: Re-emergence of coronavirus (Review). Int J Mol Med, 2020. 45(6): p. 1631-1643.
3. Needham, D. and A. Aniket, DU6187PROV. Precipitation of hydrophobic drugs as nanocrystals for injection to a subject and methods of using same, Filing Date: May 7, 2020 Application No. 63/021,241, in USPO. 2019, Duke University: USA.
4. Goulding, J. Virus Replication. 2021; Available from: https://www.immunology.org/public-information/bitesized-immunology/pathogens-and-disease/virus-replication.
5. van Tonder, E. C., et al., Preparation and physicochemical characterization of 5 niclosamide solvates and 1 hemisolvate. AAPS PharmSciTech, 2004. 5(1): p. E12-E12.
6. Harcourt, J., et al., Isolation and characterization of SARS-CoV-2 from the first US COVID—19patient. bioRxiv, 2020: p. 2020.03.02.972935.
7. CDC, SARS-CoV-2 Variant Classifications and Definitions https://www.cdc.gov/coronavirus/2019-ncov/variants/variant-info.html. 2021.
8. Bayer, Yomesan http://home.intekom.com/pharm-bayer/yomesan.html. 2003.
9. Abrams, G. J., H. C. Seftel, and H. J. Heinz, The treatment of human tapeworm infections with ‘Yomesan’. S Afr Med J, 1963. 37: p. 6-8.
10. WHO, WHO Specifications and Evaluations for Public Health Pesticides: Niclosamide. 2002, World Health Organisation, Geneva.
11. EPA. Niclosamide. 1999; Available from: https://www3.epa.gov/pesticides/chem_search/reg_actions/reregistration/fs_PC-077401_1-Nov-99.pdf.
12. Needham, D., et al., Bottom up design of nanoparticles for anti-cancer diapeutics. Journal of Drug Targeting, 2016. 24(9): p. 836-856.
13. Reddy, G., B., et al., Preclinical Testing of a Novel Niclosamide Stearate Prodrug Therapeutic (NSPT) shows efficacy against Osteosarcoma. Molecular Cancer Therapeutics, 2020. 19(7): p. 1448.
14. Jeon, S., et al., Identification of antiviral drug candidates against SARS-CoV-2 from FDA-approved drugs. bioRxiv, 2020: p. 2020.03.20.999730.
15. Jeon, S., et al., Identification of Antiviral Drug Candidates against SARS-CoV-2 from FDA-Approved Drugs. Antimicrobial agents and chemotherapy, 2020. 64(7): p. e00819-20.
16. Needham, D., Current Provisional Patent Portfolio USPO:
a) DU7305PROV, U.S. 63/111,184 “ANTI-VIRAL NICLOSAMIDE COMPOSITIONS AS VIROSTATIC THERAPEUTICS AND METHODS OF MAKING AND USING SAME” (filed Nov. 9, 2020)
b) DU7185PROV, U.S. 63/049,160, “NICLOSASPRAY” AND “NICLOSADROPS” FOR BUCCAL/THROAT, NASAL, AND EYE DROPS AND METHODS OF USING SAME” (filed Jul. 8, 2020)
c) DU7175PROV, U.S. 63/049,153, “ANTIVIRAL NICLOSAMIDE COMPOSITIONS AS VIROSTATIC THERAPEUTICS AND METHODS OF MAKING AND USING SAME” (filed Jul. 8, 2020)
d) DU7109PROV, U.S. 63/021,250, “COMPOSITIONS COMPRISING NICLOSAMIDE AND METHODS OF USING SAME AS PROPHYLAXIS AND TREATMENT OF VIRAL INFECTIONS” (filed May 7, 2020)
e) DU6187PROV, U.S. 63/021,241, PRECIPITATION OF HYDROPHOBIC DRUGS AS NANOCRYSTALS FOR INJECTION TO A SUBJECT AND METHODS OF USING SAME (filed May 7, 2020)
f) DU7099PROV, U.S. 63/014,748, “COMPOSITIONS COMPRISING NICLOSAMIDE AND METHODS OF MAKING AND USING SAME FOR THE PROPHYLAXIS AND TREATMENT OF VIRAL INFECTIONS” (filed Apr. 24, 2020)
g) DU7065PROV2, U.S. 63/111,785, “COMPOSITIONS AND FORMULATIONS COMPRISING NICLOSAMIDE AND METHODS OF MAKING AND USING SAME FOR THE TREATMENT OF VIRAL INFECTIONS” (filed Apr. 17, 2020)
h) DU7065PROV, U.S. 63/006,527, “NICLOSAMIDE COMPOSITIONS AND METHODS OF USING SAME FOR THE TREATMENT OF VIRAL INFECTIONS” (filed Apr. 7, 2020)
i) DU7308PROV, U.S. 63/007,007, “ANTI-VIRAL NICLOSAMIDE COMPOSITIONS AS VIROSTATIC THERAPEUTICS AND METHODS OF MAKING AND USING SAME” (filed Mar. 26, 2020). 2020, Duke University: USA.
17. van Tonder, E. C., et al., Preparation and physicochemical properties of niclosamide anhydrate and two monohydrates. International Journal of Pharmaceutics, 2004. 269(2): p. 417-432.
18. Braga, L., et al., Drugs that inhibit TMEM16 proteins block SARS-CoV-2 spike-induced syncytia. Nature, 2021. 594(7861): p. 88-93.
19. Cabrita, I., et al., Niclosamide repurposed for the treatment of inflammatory airway disease. JCI Insight, 2019. 4(15).
20. Cheng, B., et al., Niclosamide induces protein ubiquitination and inhibits multiple pro-survival signaling pathways in the human glioblastoma U-87 MG cell line. PloS one, 2017. 12(9): p. e0184324-e0184324.
21. Imperi, F., et al., New life for an old drug. the anthelmintic drug niclosamide inhibits Pseudomonas aeruginosa quorum sensing. Antimicrobial Agents and Chemotherapy, 2013. 57(2): p. 996.
22. Jurgeit, A., et al., Niclosamide Is a Proton Carrier and Targets Acidic Endosomes with Broad Antiviral Effects. PLOS Pathogens, 2012. 8(10): p. e1002976.
23. Kadri, H., O. A. Lambourne, and Y. Mehellou, Niclosamide, a Drug with Many (Re)purposes. ChemMedChem, 2018. 13(11): p. 1088-1091.
24. Khanim, F. L., et al., Redeployment-based drug screening identifies the anti-helminthic niclosamide as anti-myeloma therapy that also reduces free light chain production. Blood Cancer Journal 2011. 1: p. e39.
25. Liu, C., et al., Niclosamide inhibits androgen receptor variants expression and overcomes enzalutamide resistance in castration-resistant prostate cancer. Clin Cancer Res, 2014. 20(12): p. 3198-3210.
26. McCollum, C. W., et al., Identification of vascular disruptor compounds by analysis in zebrafish embryos and mouse embryonic endothelial cells. Reproductive toxicology (Elmsford, N.Y.), 2017. 70: p. 60-69.
27. Miner, K., et al., Drug Repurposing. The Anthelmintics Niclosamide and Nitazoxanide Are Potent TMEM16A Antagonists That Fully Bronchodilate Airways. Frontiers in Pharmacology, 2019. 10(51).
28. Mook, R. A., S. Zhao, and W. Chen, Abstract 5491: Interrogating the mechanism of Wnt pathway inhibition by niclosamide. Cancer Research, 2014. 74(19 Supplement): p. 5491.
29. Mostafa, A., et al., FDA-Approved Drugs with Potent In Vitro Antiviral Activity against Severe Acute Respiratory Syndrome Coronavirus 2. Pharmaceuticals, 2020. 13(12).
30. Prabhakara, C., et al., Niclosamide inhibits SARS-CoV2 entry by blocking internalization through pH-dependent CLIC/GEEC endocytic pathway. bioRxiv, 2020: p. 2020.12.16.422529.
31. Schweizer, M. T., et al., A phase I study of niclosamide in combination with enzalutamide in men with castration-resistant prostate cancer. PLOS ONE, 2018. 13(6): p. e0198389.
32. Xu, J., et al., Broad Spectrum Antiviral Agent Niclosamide and Its Therapeutic Potential. ACS infectious diseases, 2020. 6(5): p. 909-915.
33. Zimmerman, J. J., A. von Saint André-von Arnim, and J. McLaughlin, Chapter 74—Cellular Respiration, in Pediatric Critical Care (Fourth Edition), B. P. Fuhrman and J. J. Zimmerman, Editors. 2011, Mosby: Saint Louis. p. 1058-1072.
34. Ko, M., et al., Comparative analysis of antiviral efficacy of FDA-approved drugs against SARS-CoV-2 in human lung cells: Nafamostat is the most potent antiviral drug candidate. bioRxiv, 2020: p. 2020.05.12.090035.
35. clinicaltrials.gov, COVID19 Niclosamide https://clinicaltrials.gov./ct2/results?term=Niclosamide&cond=Covid19. 2021.
36. Daewoong-Pharmaceutical. Daewoong Pharmaceutical Partners with Tufts Medical Center for Phase 2 Clinical Trial with Niclosamide. https://www.prnewswire.com/news-releases/daewoong-pharmaceutical-partners-with-tufts-medical-center-for-phase-2-clinical-trial-with-niclosamide-301162739.html. 2020.
37. Union-Therapeutics, UNION's nasal spray for COVID-19 enters a 1,500 high-risk patient study. 2021.
38. Pubmed, https://pubmed.ncbi.nlm.nih.gov/?term=niclosamide&timeline=expanded. 2021.
39. Terada, H., Uncouplers of oxidative phosphorylation. Environmental health perspectives, 1990. 87: p. 213-218.
40. CHANG, Y.-W., et al., Pharmacokinetics of Anti-SARS-CoV Agent Niclosamide and Its Analogs in Rats. Journal of Food and Drug Analysis, 2006. 14(4): p. 329-333.
41. Gassen, N.C., et al., Analysis of SARS-CoV-2-controlled autophagy reveals spermidine, MK-2206, and niclosamide as putative antiviral therapeutics. bioRxiv, 2020: p. 2020.04.15.997254.
42. Pindiprolu, S. K. S. S. and S. H. Pindiprolu, Plausible mechanisms of Niclosamide as an antiviral agent against COVID-19. Medical Hypotheses, 2020. 140: p. 109765.
43. Wu, C.-J., et al., Inhibition of severe acute respiratory syndrome coronavirus replication by niclosamide. Antimicrobial agents and chemotherapy, 2004. 48(7): p. 2693-2696.
44. Zhang, X. W. and Y. L. Yap, Old drugs as lead compounds for a new disease? Binding analysis of SARS coronavirus main proteinase with HIV, psychotic and parasite drugs. Bioorganic & medicinal chemistry, 2004. 12(10): p. 2517-2521.
45. Kim, S., et al., Effect of Niclosamide on cytotoxicity and cell viability in small airway epithelial cells in preparation, 2021.
46. Laise, P., et al., The Host Cell ViroCheckpoint: Identification and Pharmacologic Targeting of Novel Mechanistic Determinants of Coronavirus-Mediated Hijacked Cell States. bioRxiv: the preprint server for biology, 2020: p. 2020.05.12.091256.
47. Chen, W., et al., Niclosamide: Beyond an antihelminthic drug. Cellular Signalling, 2018. 41: p. 89-96.
48. Barini, E., et al., The Anthelmintic Drug Niclosamide and Its Analogues Activate the Parkinson's Disease Associated Protein Kinase PINK1. Chembiochem: a European journal of chemical biology, 2018. 19(5): p. 425-429.
49. Li, Y., et al., Multi-targeted therapy of cancer by niclosamide: A new application for an old drug. Cancer Letters, 2014. 349(1): p. 8-14.
50. Pan, J.-X., K. Ding, and C.-Y. Wang, Niclosamide, an old antihelminthic agent, demonstrates antitumor activity by blocking multiple signaling pathways of cancer stem cells. Chinese Journal of Cancer, 2012. 31(4): p. 178-184.
51. Li, Z., et al., Niclosamide inhibits the proliferation of human osteosarcoma cell lines by inducing apoptosis and cell cycle arrest. Oncology reports, 2015. 33(4): p. 1763-1768.
52. Osada, T., et al., Antihelminth Compound Niclosamide Downregulates Wnt Signaling and Elicits Antitumor Responses in Tumors with Activating APC Mutations. Cancer Research, 2011. 71(12): p. 4172-4182.
53. Han, Z., et al., Niclosamide Induces Cell Cycle Arrest in G1 Phase in Head and Neck Squamous Cell Carcinoma Through Let-7d/CDC34 Axis. Frontiers in pharmacology, 2019. 9: p. 1544-1544.
54. Lee, M.-C., et al., Niclosamide inhibits the cell proliferation and enhances the responsiveness of esophageal cancer cells to chemotherapeutic agents. Oncology reports, 2020. 43(2): p. 549-561.
55. Ahmed, K., et al., A Second WNT for Old Drugs: Drug Repositioning against WNT-Dependent Cancers. Cancers, 2016. 8(7): p. 66.
56. Arend, R. C., et al., The Wnt/β-catenin pathway in ovarian cancer: a review. Gynecol Oncol, 2013. 131(3): p. 772-9.
57. Chen, M., et al., The anti-helminthic niclosamide inhibits Wnt/Frizzled1 signaling. Biochemistry, 2009. 48(43): p. 10267-74.
58. Suliman, M. A., et al., Niclosamide inhibits colon cancer progression through downregulation of the Notch pathway and upregulation of the tumor suppressor miR-200family. International journal of molecular medicine, 2016. 38(3): p. 776-784.
59. Eward, W., S. Suter, and D. Needham. Testing a New Prodrug Nanoparticle Formulation of Niclosamide as a Novel Therapeutic for Osteosarcoma. in 4th Annual Consortium for Comparative Oncology (C30) Symposium. 2019. Durham Convention Center, Durham, NC.
60. Mirabelli, C., et al., Morphological Cell Profiling of SARS-CoV-2 Infection Identifies Drug Repurposing Candidates for COVID-19. bioRxiv, 2020: p. 2020.05.27.117184.
61. Zhu, N., et al., Morphogenesis and cytopathic effect of SARS-CoV-2 infection in human airway epithelial cells. Nature Communications, 2020. 11(1): p. 3910.
62. Fehr, A. R. and S. Perlman, Coronaviruses: an overview of their replication and pathogenesis. Methods in molecular biology (Clifton, N.J.), 2015. 1282: p. 1-23.
63. Huang, Y., et al., SARS-CoV-2 Viral Load in Clinical Samples from Critically Ill Patients. Am J Respir Crit Care Med, 2020. 201(11): p. 1435-1438.
64. Shang, J., et al., Cell entry mechanisms of SARS-CoV-2. Proc Natl Acad Sci USA, 2020. 117(21): p. 11727-11734.
65. Jung, E., et al., Neutralization of Acidic Intracellular Vesicles by Niclosamide Inhibits Multiple Steps of the Dengue Virus Life Cycle In Vitro. Scientific Reports, 2019.9(1): p. 8682.
66. Chang, C.-W., et al., Increased ATP generation in the host cell is required for efficient vaccinia virus production. Journal of biomedical science, 2009. 16(1): p. 80-80.
67. Nagy, P. D. and W. Lin, Taking over Cellular Energy-Metabolism for TBSV Replication: The High ATP Requirement of an RNA Virus within the Viral Replication Organelle. Viruses, 2020. 12: p. 56.
68. CDC. Symptoms of coronavirus, https://www.cdc.gov/cornavirus/2019-ncov/symptoms-testing/symptoms.html. 2021; Available from: https://www.cdc.gov/coronavirus/2019-ncov/symptoms-testing/symptoms.html.
69. CDC, COVID-19 Vaccine Breakthrough Infections Reported to CDC—United States, Jan. 1-Apr. 30, 2021, https://www.cdc.gov/mmwr/volumes/70wr/mm7021e3.htm. 2021.
70. McNamara, D., Delta Variant Key to Breakthrough Infections in Vaccinated Israelis. 2021, Medscape.
71. COVID19_help.org. Niclosamide. 2021; Available from: https://covid19-help.org/substance/niclosamide.
72. AzurRx-BioPharma, AzurRx BioPharma Initiates Phase 2 Clinical Trial of Niclosamide for the Treatment of COVID-19 Gastrointestinal Infections. 2021, Biospace.
73. Fallingborg, J., Intraluminal pH of the human gastrointestinal tract. Dan Med Bull, 1999. 46(3): p. 183-96.
74. Yu, S., et al., Niclosamide-Clay Intercalate Coated with Nonionic Polymer for Enhanced Bioavailability toward COVID-19 Treatment. Polymers (Basel), 2021. 13(7).
75. Hyundai_Bioscience, Hyundai Bioscience: Niclosamide-based CP-COV03, an Oral Antiviral Drug May Be a Problem Solver for COVID-19. 2021, Newswire.
76. Nair, A. B. and S. Jacob, A simple practice guide for dose conversion between animals and human. J Basic Clin Pharm, 2016. 7(2): p. 27-31.
77. Hobson, J. J., et al., Scalable nanoprecipitation of niclosamide and in vivo demonstration of long-acting delivery after intramuscular injection. Nanoscale, 2021. 13(13): p. 6410-6416.
78. TFF-Pharmaceuticals. “Science” https://tffpharma.com/science/. 2020.
79. Union-Therapeutics. UNION therapeutics A/S completes dosing of healthy volunteers with UNI911 (inhaled niclosamide) for COVID-19. 2020; Available from: https://www.uniontherapeutics.com/news-events/news/union-therapeutics-a-s-completes-dosing-of-healthy-volunteers-with-uni911-inhaled-niclosamide-for-covid-19.
80. Reijenga, J., et al., Development of Methods for the Determination of pKa Values. Analytical chemistry insights, 2013. 8: p. 53-71.
81. ACDILabs, Niclosamide. 2020.
82. FAO, Food and Agriculture Organization of the United Nations (FAO) SPECIFICATIONS AND EVALUATIONS FOR AGRICULTURAL PESTICIDES NICLOSAMIDE 2′,5-dichloro-4′-nitrosalicylanilide 2004.
83. EPA, ECOTOXicology Knowledgebase (ECOTOA). 2021.
84. Tran, T. H., J. Labanowski, and H. Gallard, Adsorption and transformation of the anthelmintic drug niclosamide by manganese oxide. Chemosphere, 2018. 201: p. 425-431.
85. McConville, M. B., T. D. Hubert, and C. K. Remucal, Direct Photolysis Rates and Transformation Pathways of the Lampricides TFM and Niclosamide in Simulated Sunlight. Environmental Science & Technology, 2016. 50(18): p. 9998-10006.
86. Cameselle, J., J. Meireles Ribeiro, and A. Sillero, Derivation and use of a formula to calculate the net charge of acid-base compounds. Its application to amino acids, proteins and nucleotides. Biochemical Education, 1986. 14(3): p. 131-136.
87. Graebing, P. W., et al., Metabolism of Niclosamide in Sediment and Water Systems. Journal of Agricultural and Food Chemistry, 2004. 52(19): p. 5924-5932.
88. Caira, M. R., et al., Diverse Modes of Solvent Inclusion in Crystalline Pseudopolymorphs of the Anthelmintic Drug Niclosamide. Journal of inclusion phenomena and molecular recognition in chemistry, 1998. 31(1): p. 1-16.
89. de Villiers, M. M., et al., Comparison of the physical and chemical stability of niclosamide crystal forms in aqueous versus nonaqueous suspensions. Drug Dev Ind Pharm, 2004. 30(6): p. 581-92.
90. Tomlin, C. D. S., The Pesticide Manual—World Compendium. 10th edition. 1994, Surrey, UK: The British Crop Protection Council.
91. Brunaugh, A. D., et al., Development and evaluation of inhalable composite niclosamide-lysozyme particles: A broad-spectrum, patient-adaptable treatment for coronavirus infections and sequalae. PloS one, 2021. 16(2): p. e0246803-e0246803.
92. Gobas, F. A. P. C., et al., A novel method for measuring membrane-water partition coefficients of hydrophobic organic chemicals: Comparison with 1-octanol-water partitioning. Journal of Pharmaceutical Science $V 77, 1988(3): p. 265-272.
93. Paul, A. D., et al., Modeling the oral cavity with mucoadhesive drug delivery systems—a potential alternative to conventional therapy. International Journal of Pharmaceutical Sciences and Drug Research, 2017. 9: p. 299-307.
94. Baldwin, C. M. and L. J. Scott, Mometasone furoate: a review of its intranasal use in allergic rhinitis. Drugs, 2008. 68(12): p. 1723-39.
95. FDA, Approval Package for Nasonex, (mometasone furoate monohydrate) NDA 020762/S-051 Feb. 24, 2015, CENTER-FOR-DRUG-EVALUATION-AND-RESEARCH, Editor. 2015.
96. Merck, Prescribing information Nasonex. 1997.
97. Chaudry, I. A., Fluticasone propionate nasal pharmaceutical formulations and methods of using same U.S. Ser. No. 11/250,220, USPO, Editor. 2011, Mylan Specialty LP.
98. Nimmagadda, S. R., et al., Fluticasone Propionate Results in Improved Glucocorticoid Receptor Binding Affinity and Reduced Oral Glucocorticoid Requirements in Severe Asthma. Annals of Allergy, Asthma & Immunology, 1998. 81(1): p. 35-40.
99. Shah, B. A. K., FLONASE (fluticasone propionate) nasal spray. Prescribing information, DPARP, Editor. 2019.
100. Jones, N., The nose and paranasal sinuses physiology and anatomy. Advanced Drug Delivery Reviews, 2001. 51(1): p. 5-19.
101. Maltas, E., Binding interactions of niclosamide with serum proteins. Journal of Food and Drug Analysis, 2014. 22(4): p. 549-555.
102. Yeung, L. and D. Needham, Binding Properties of Niclosamide to Bovine Serum Albumin. (in preparation). 2021, Duke University.
103. AATbio.com. Trizma® Buffer (pH 7.0 to 9.2) Preparation and Recipe. 2021; Available from: https://www.aatbio.com/resources/buffer-preparations-and-recipes/trizma-buffer-ph-7-2-to-9-0.
104. Li, A. and S. H. Yalkowsky, Solubility of Organic Solutes in Ethanol/Water Mixtures. Journal of Pharmaceutical Sciences, 1994. 83(12): p. 1735-1740.
105. Yalkowsky, S. H., ed. Techniques of Solubilization of Drugs. 1981, Marcel Dekker: NY.
106. Yalkowsky, S. H. and T. J. Roseman, Solubilization of Drugs by Co-Solvents., in Techniques of Solubilization of Drugs, S. H. Yalkowsky, Editor. 1981, Marcel Dekker Inc.: New York. p. 91-134.
107. Bayer. YOMESAN® tablets (MSDS). 2008; Available from: https://www.bayer.co.za/static/documents/MSDS/PIs/YOMESAN_EN_PI.pdf.
108. Washington, N., et al., Determination of baseline human nasal pH and the effect of intranasally administered buffers. Int J Pharm, 2000. 198(2): p. 139-46.
109. Epstein, P. S. and M. S. Plesset, On the Stability of Gas Bubbles in Liquid-Gas Solutions. THE JOURNAL OF CHEMICAL PHYSICS, 1950. 18 (11): p. 1505-1409.
110. Duncan, P. B. and D. Needham, Test of the Epstein-Plesset model for gas microparticle dissolution in aqueous media: Effect of surface tension and gas undersaturation in solution. Langmuir, 2004. 20(7): p. 2567-2578.
111. Duncan, P. B. and D. Needham, Microdroplet dissolution into a second-phase solvent using a micropipet technique: Test of the Epstein-Plesset model for an aniline-water system. Langmuir, 2006. 22(9): p. 4190-4197.
112. Su, J. T., P. Brent Duncan, Amit Momaya, Arimatti Jutila, and David Needham The Effects of Hydrogen Bonding on the Diffusion of Water in n-Alkanes and n-Alcohols Measured with a Novel Single Microdroplet Method. J. Chem Phys, 2010. 132 (4): p. 044506 (1-8).
113. Su, J. T. and D. Needham, Mass Transfer in the Dissolution of a Multicomponent Liquid Droplet in an Immiscible Liquid Environment. Langmuir, 2013. 29(44): p. 13339-13345.
114. Utoft, A., et al., Manipulating Single Microdroplets of NaCl Solutions: Solvent Dissolution, Microcrystallization, and Crystal Morphology. Langmuir, 2018. 34(12): p. 3626-3641.
115. Kim, D. H., et al., Mechanical properties and microstructure of polycrystalline phospholipid monolayer shells: Novel solid microparticles. Langmuir, 2003. 19(20): p. 8455-8466.
116. Kim, D. H., et al., Viscoelastic properties of phospholipid monolayer shells on air microbubbles. Biophysical Journal, 1998. 74(2): p. A313-A313.
117. Wölfel, R., et al., Virological assessment of hospitalized patients with COVID-2019. Nature, 2020. 581(7809): p. 465-469.
118. Hou, Y. J., et al., SARS-CoV-2 Reverse Genetics Reveals a Variable Infection Gradient in the Respiratory Tract. Cell, 2020.
119. Guy, J. S., et al., Characterization of a coronavirus isolated from a diarrheic foal. J Clin Microbiol, 2000. 38(12): p. 4523-6.
120. Belouzard, S., et al., Mechanisms of coronavirus cell entry mediated by the viral spike protein. Viruses, 2012. 4(6): p. 1011-1033.
121. Puchelle, E., et al., Airway Epithelial Repair, Regeneration, and Remodeling after Injury in Chronic Obstructive Pulmonary Disease. Proceedings of the American Thoracic Society, 2006. 3(8): p. 726-733.
122. Olmsted, S. S., et al., Diffusion of Macromolecules and Virus-Like Particles in Human Cervical Mucus. Biophysical Journal, 2001. 81(4): p. 1930-1937.
123. Beule, A. G., Physiology and pathophysiology of respiratory mucosa of the nose and the paranasal sinuses. GMS current topics in otorhinolaryngology, head and neck surgery, 2010. 9: p. Doc07-Doc07.
124. Liesman, R. M., et al., RSV-encoded NS2 promotes epithelial cell shedding and distal airway obstruction. The Journal of Clinical Investigation, 2014. 124(5): p. 2219-2233.
125. Robinot, R., et al., SARS-CoV-2 infection induces the dedifferentiation of multiciliated cells and impairs mucociliary clearance. Nature Communications, 2021. 12(1): p. 4354.
126. Sims, A. C., et al., Severe acute respiratory syndrome coronavirus infection of human ciliated airway epithelia: role of ciliated cells in viral spread in the conducting airways of the lungs. Journal of virology, 2005. 79(24): p. 15511-15524.
127. Elmore, S., Apoptosis: a review of programmed cell death. Toxicologic pathology, 2007. 35(4): p. 495-516.
128. Needham, D., Z. Kellehery, and C. Barkouskas, Buffered solutions of niclosamide enter cells in culture and show reductions in ATP production within minutes of exposure in preparation, 2021.
129. Baliga, S., S. Muglikar, and R. Kale, Salivary pH: A diagnostic biomarker. Journal of Indian Society of Periodontology, 2013. 17(4): p. 461-465.
130. Gallo, O., et al., The central role of the nasal microenvironment in the transmission, modulation, and clinical progression of SARS-CoV-2 infection. Mucosal Immunology, 2021. 14(2): p. 305-316.
131. Boegh, M. and H. M. Nielsen, Mucus as a barrier to drug delivery—understanding and mimicking the barrier properties. Basic Clin Pharmacol Toxicol, 2015. 116(3): p. 179-86.
132. Labiris, N. R. and M. B. Dolovich, Pulmonary drug delivery. Part I. physiological factors affecting therapeutic effectiveness of aerosolized medications. Br J Clin Pharmacol, 2003. 56(6): p. 588-99.
133. Evans, M. J., et al., Cellular and molecular characteristics of basal cells in airway epithelium. Exp Lung Res, 2001. 27(5): p. 401-15.
134. Dawes, C., Estimates, from salivary analyses, of the turnover time of the oral mucosal epithelium in humans and the number of bacteria in an edentulous mouth. Archives of Oral Biology, 2003. 48(5): p. 329-336.
135. Carotti, M., et al., Chest CT features of coronavirus disease 2019 (COVID-19) pneumonia: key points for radiologists. La Radiologia medica, 2020. 125(7): p. 636-646.
136. Bernstein, L., C. Y. Johnson, and L. Mcginley, Coronavirus destroys lungs. But doctors are finding its damage in kidneys, hearts and elsewhere., in Washington Post. 2020.
137. Liu, J., et al., SARS-CoV-2 cell tropism and multiorgan infection. Cell Discovery, 2021. 7(1): p. 17.
138. Rhea, E. M., et al., The S1 protein of SARS-CoV-2 crosses the blood-brain barrier in mice. Nature Neuroscience, 2020.
139. Meinhardt, J., et al., Olfactory transmucosal SARS-CoV-2 invasion as a port of central nervous system entry in individuals with COVID-19. Nature Neuroscience, 2021. 24(2): p. 168-175.
140. Jara, M. O., et al., Niclosamide Inhalation Powder Made by Thin-Film Freezing: Pharmacokinetic and Toxicology Studies in Rats and Hamsters. bioRxiv, 2021: p. 2021.01.26.428293.
141. Brunaugh, A. D., et al., Effect of Particle Formation Process on Characteristics and Aerosol Performance of Respirable Protein Powders. Mol Pharm, 2019. 16(10): p. 4165-4180.
142. Ferrati, S., et al., Influence of Formulation Factors on the Aerosol Performance and Stability of Lysozyme Powders: a Systematic Approach. AAPS PharmSciTech, 2018. 19(7): p. 2755-2766.
143. Murgolo, N., et al., SARS-CoV-2 tropism, entry, replication, and propagation: Considerations for drug discovery and development. PLoS Pathog, 2021. 17(2): p. e1009225.
144. Saccon, E., et al., Cell-type-resolved quantitative proteomics map of interferon response against SARS-CoV-2. iScience, 2021. 24(5): p. 102420.
145. Worthing, C. R. and S. B. Walker The Pesticide Manual-A World Compendium. 8th edition. 1987, Thornton Heath, UK: The British Crop Protection Council.
146. Teleflex. Madgic laryngo-tracheal mucosal atomization device. 2020; Available from: https://www.teleflexarcatalog.com/anesthesia-respiratory/respiratory/products?utf8=%E2%9C%93&brand_id=&search=MADgiC+Laryngo-Tracheal-Mucosal-Atomization+Device.
147. Needham, D., P. Lee, and S. Kim, Rational Design of Nasal and Throat Sprays for Respiratory Viral Disease: 5 Prototypical Formulations of Niclosamide for Cell ATP, Drug Deposition, Viral Activity and Therapeutic Index in Airway Cells. 2021, NIH R21: Duke University.
148. Barkauskas, C. and D. Needham, DU7185 PROV. “Niclosaspray” and “niclosadrops” for buccal/throat, nasal, and eye drops and methods of using same, Filing Date Jul. 8, 2020, Application No. U.S. 63/049,160. 2020, Duke University.
149. Needham, D., DU7065 PROV. Niclosamide compositions and methods of using same for the treatment of viral infections, Filing Date: Apr. 7, 2020, Application No. 63/006,527, USPO, Editor. 2020, Duke University: USA.
150. Needham, D., DU7305 PROV. Anti-viral niclosamide compositions as virostatic therapeutics and methods of making and using same” Filing Date Nov. 9, 2020, Application No. U.S. 63/111,184. 2020, Duke University.
151. Needham, D., DU7099 PROV. Compositions comprising niclosamide and methods of making and using same for the prophylaxis and treatment of viral infections, Filing date: Apr. 24, 2020 Application No. U.S. 63/014,748, USPO, Editor. 2020, Duke University: USA.
152. Needham, D., DU7065.2 PROV. compositions and formulations comprising niclosamide and methods of making and using same for the treatment of viral infections Filing date: Apr. 17, 2020, Application No. 63/011,785, USPO, Editor. 2020, Duke University: USA.
153. Needham, D., DU7308 PROV. Antiviral niclosamide compositions as virostatic therapeutics and methods of making and using same Filing Date: Mar. 26, 2020, Application No. U.S. 63/00,007, USPO, Editor. 2020, Duke University: USA.
154. Needham, D., DU7109 PROV. anti-viral niclosamide compositions as virostatic therapeutics and methods of making and using same. Filing Date May 7, 2020, Application No. U.S. 63/021,250. 2020, Duke University, to be licensed to David Needham at Bio-Gyali LLC.
155. Needham, D., DU7175 PROV. Antiviral niclosamide compositions as virostatic therapeutics and methods of making and using same, Filing Date Jul. 8, 2020, Application No. U.S. 63/049,153. 2020, Duke University.
156. Needham, D., DU6187 PROV. Precipitation of hydrophobic drugs as nanocrystals for injection to a subject and methods of using same, Filing Date May 7, 2020, Application No. U.S. 63/021,241. 2020.
157. Thiam, A. R. and I. Dugail, Lipid droplet-membrane contact sites—from protein binding to function. J Cell Sci, 2019. 132(12).
158. Nardacci, R., et al., Evidences for lipid involvement in SARS-CoV-2 cytopathogenesis. Cell Death & Disease, 2021. 12(3): p. 263.
159. Barkauskas, C. and D. Needham, Niclosamide spray as a prophylactic and early treatment for SARS-CoV-2. 2021, American Lung Association: Duke University.
160. Barkauskas, C. and D. Needham, Niclosamide-Based Nasal and Throat Spray Prophylaxis and Early Treatment of COVID19 and Other Respiratory Viral Infections. 2021, 2022 Harrington Scholar-Innovator Grant: Duke University.
161. Fujii, G., SBIR: Nasal and Throat Spray Formulations of Niclosamide for Pre- and Post-Exposure Prophyalaxis of Respiratory Viral Infections. 2021, NIH NIAID: Molecular Express, Inc, CA.
162. Ourworldindata, Share of people vaccinated against COVID-19, Jul. 23, 2021. 2021.
163. CDC, Older Adults, C.f.D.C.a. Prevention, Editor. 2021.
164. CDC, Daily Activities and Going Out, https://www.cdc.gov/coronavirus/2019-ncov/daily-life-coping/going-out.html. 2021.
165. CDC, Travel https: www.cdc.gov/coronavirus/2019-ncov/travelers/travel-during-covid19.html. 2021.
166. CDC, COVID-19 Vaccines for Children and Teens, https://www.cdc.gov/coronavirus/2019-ncov/vaccines/recommendations/adolescents.html. 2021.
167. CDC, Vaccine Information for People with Certain Medical Conditions, https://www.cdc.gov/coronavirus/2019-ncov/need-extra-precautions/people-with-medical-conditions.html. 2021.
168. CDC, Interim List of Categories of Essential Workers Mapped to Standardized Industry Codes and Titles. 2021.
169. Needham, D., Development of clinically effective formulations for anticancer applications: why it is so difficult?, in Biomaterials for Cancer Therapeutics, Evolution and Innovation, K. Park, Editor. 2020, Woodhead Publishing: Cambridge, UK.

What is claimed is:

1. A composition for application to a mucus membrane of a patient, comprising:
1. a buffered aqueous solution comprising about 10 uM to about 300 uM niclosamide, wherein the buffered aqueous solution has a pH between about pH 7.0 and about pH 9.2.

2. The composition of claim 1, wherein the buffered aqueous solution has a pH between about pH 8.0 and about pH 9.0.

3. The composition of claim 1, comprising about 20 uM to about 40 uM niclosamide.

4. The composition of claim 1, wherein the buffered aqueous solution comprises a Tris(hydroxymethyl)aminomethane buffer or a 4-(2-hydroxyethyl)-1-piperazineethanesulfonic acid (HEPES) buffer.

5. The composition of claim 1, further comprising about 0.5% (v/v) to about 2% (v/v) carboxymethyl cellulose.

6. The composition of claim 1, further comprising about 1% (v/v) to about 2% (v/v) ethanol.

7. The composition of claim 1, formulated as a nasal spray, an oral-throat spray, a nasal rinse, or an oral-throat rinse.

8. A method of reducing a viral infection comprising:
2. administering a composition comprising a buffered aqueous solution comprising about 10 uM to about 300 uM niclosamide to a mucus membrane of a person, wherein the buffered aqueous solution has a pH between 7.0 and pH 9.2.

9. The method of claim 8, wherein the composition is administered to a nasal passage of the person.

10. The method of claim 8, wherein the composition is administered to the patient's throat.

11. The method of claim 9, wherein the composition is administered as a nasal spray or a nasal rinse.

12. The method of claim 10, wherein the composition is administered as an oral-throat spray or as an oral-throat rinse.

13. The method of claim 8, wherein the buffered aqueous solution has a pH between about pH 8.0 and about pH 9.2.

14. The method of claim 8, wherein the composition comprises about 20 uM to about 40 uM niclosamide.

15. The method of claim 8, wherein the buffered aqueous solution comprises a Tris(hydroxymethyl)aminomethane buffer or a 4-(2-hydroxyethyl)-1-piperazineethanesulfonic acid (HEPES) buffer.

16. The method of claim 8, wherein the composition further comprises about 0.5% (v/v) to about 2% (v/v) carboxymethyl cellulose.

17. The method of claim 8, wherein the composition further comprises about 1% (v/v) to about 2% (v/v) ethanol.

18. The method of claim 8, wherein the viral infection is a coronavirus infection.

19. The method of claim 18, wherein the coronavirus viral infection is from 229E (alpha coronavirus), NL63 (alpha coronavirus), OC43 (beta coronavirus), HKU1 (beta coronavirus), MERS-CoV (Middle East Respiratory Syndrome (MERS)), SARS-CoV (severe acute respiratory syndrome, (SARS)) or SARS-CoV-2 (coronavirus disease 2019 (COVID-19)).

20. The method of claim 18, wherein the coronavirus infection is a coronavirus variant respiratory virus infection.

21. The method of claim 18, wherein the coronavirus infection is from SARS-CoV-2 (coronavirus disease 2019 (COVID-19)).

22. The method of claim 8, wherein replication of viral particles of the viral infection is reduced by at least 50%, compared to an untreated viral infection.

23. The method of claim 21, wherein the replication of viral particles is reduced within about 1 hour following the administration.

24. A method of preparing a niclosamide composition, comprising injecting an ethanolic niclosamide solution into a buffered aqueous solution to produce the niclosamide composition in the buffered solution.

25. The method of claim 24, wherein the ethanolic niclosamide solution is 100 uL of a 2 mM niclosamide and is injected into 10 mL of a pH 9.2 buffered aqueous solution to make a 20 uM solution of niclosamide by diluting the initial ethanolic niclosamide in a 1:99 v/v ratio.

26. The method of claim 24, wherein the ethanolic niclosamide solution is 10 mL of a 2 mM niclosamide and is injected into 1 L of a pH 9.2 buffered aqueous solution to make 1 L of a 20 uM solution of niclosamide by diluting the initial ethanolic niclosamide in a 1:99 v/v ratio.

27. The method of claim 24, wherein the ethanolic niclosamide solution is 100 uL of a 30 mM niclosamide and is injected into 10 mL of a pH 9.2 buffered aqueous solution to make a 300 uM solution of niclosamide by diluting the initial ethanolic niclosamide in a 1:99 v/v ratio.

28. The method of claim 24, wherein the ethanolic niclosamide solution is 10 mL of a 30 mM niclosamide and is injected into 1 L of a pH 9.2 buffered aqueous solution to make 1 L of a 300 uM solution of niclosamide by diluting the initial ethanolic niclosamide in a 1:99 v/v ratio

29. The method of claim 24, wherein the niclosamide composition is prepared under sterile conditions, filled into vials or bottles, capped and sealed.

30. A method for preparing a niclosamide composition, comprising crushing niclosamide tablets into a powder and incubating them in a buffered aqueous solution to extract niclosamide.

31. The method of claim 30 where the buffered aqueous solution has a pH of pH 7 to pH 9.5

32. The method of claim 30 where the niclosamide tablet is 500 mg and the amount of buffered aqueous solution is 10 mL.

33. The method of claim 30, further comprising filtering the niclosamide composition through a 0.22 um filter.

34. The method of claim 30, where the pH of the buffered aqueous solution is pH 9.34 and the final concentration of extracted niclosamide is 348 uM.
